(19) United States
(12) Patent Application Publication
(10) Pub. No.: US 2019/0374567 A1
(43) Pub. Date: Dec. 12, 2019

(54) Reverse Transcriptase Dependent Conversion of RNA Templates Into DNA

(71) Applicant: Aftab S. Karim, Farmington, NY (US)

(72) Inventor: Aftab S. Karim, Farmington, NY (US)

(21) Appl. No.: 16/140,140

(22) Filed: Sep. 24, 2018

Related U.S. Application Data
(63) Continuation-in-part of application No. PCT/US17/23980, filed on Mar. 24, 2017.
(60) Provisional application No. 62/313,031, filed on Mar. 24, 2016.

(51) Int. Cl. A61K 31/7105; C12N 15/85; C12N 15/113

(52) U.S. Cl. CPC A61K 31/7105; A61K 45/06; C12N 15/113; C12N 15/85

(57) ABSTRACT

The function of reverse transcriptase can be utilized to convert synthetic RNA templates into double stranded DNA for including but not limited to therapeutic function; to aid in diagnosis; to clear infection in latent cell populations such as memory T-cells; and prevent infection in exposed individuals by delivery of the RNA templates/primers to cell populations prone to infection, such as CD4 T-cells.

### CROSS-REFERENCE TO RELATED APPLICATIONS

This application is a continuation-in-part of PCT Application Serial No. PCT/US2017/023980, filed Mar. 24, 2017, which claims priority to U.S. Provisional Application Ser. No. 62/313,031, filed Mar. 24, 2016, both of which are incorporated by reference herein in their entirety.

### FIELD

The disclosure relates generally to reverse transcription. The disclosure relates specifically to utilizing reverse transcription in cells.

### BACKGROUND

Conditions linked to retroviruses included AIDS, cancer, leukemia, various sexually transmitted diseases, tropical spastic paresis, and HTLV-1-associated myelopathy. Retroviruses are classified as lentiviruses, oncoviruses, or spumaviruses. HIV (a lentivirus) and human T-cell leukemia virus (an oncovirus) are retroviruses and contain a reverse transcriptase. Reverse transcriptases have been targeted with inhibitors to slow the progression of retroviral diseases.

It would be advantageous to target a sequence encoding a therapeutic or diagnostic substance directly to cells in which the virus is present.

### SUMMARY

An embodiment of the disclosure is a method for treating a disorder in a mammal comprising administering to the mammal a composition comprising (a) an aviral RNA template comprising: (i) a primer binding sequence at the 3′ end of the RNA template; (ii) a sequence encoding a gene; (iii) a sequence encoding a promoter that is capable of regulating expression of the gene; wherein the aviral RNA template serves as a template for the synthesis of a complementary single stranded DNA by reverse transcriptase in cells expressing reverse transcriptase; wherein the single stranded DNA serves as a template for synthesis of a second complementary strand of DNA by a DNA polymerase; thus resulting in a double stranded DNA; wherein the complementary single stranded DNA is not synthesized from the aviral RNA template in cells lacking reverse transcriptase; and (b) a first primer complementary to the RNA template; wherein the administration of the RNA template is through an aviral delivery technique; wherein the mammal comprises at least one cell expressing reverse transcriptase; and wherein the RNA template encodes a substance capable of treating the disorder. In an embodiment, the DNA polymerase is reverse transcriptase. In an embodiment, the RNA template lacks a poly A tail. In an embodiment, the RNA template has a length of at least three hundred bases. In an embodiment, the RNA template includes no more than five viral genes. In an embodiment, the RNA template is annealed to the primer prior to administration to the mammal. In an embodiment, the primer is not annealed to the RNA template. In an embodiment, a second primer either produced in the cells or administered to the mammal primes the synthesis of the second strand of DNA either by reverse transcriptase or other DNA polymerase. In an embodiment, the RNA template encodes a polyadenylation signal. In an embodiment, the RNA template is reverse transcribed to yield a single strand DNA and then the single strand DNA is further transcribed by a DNA polymerase to yield a double strand DNA. In an embodiment, the DNA polymerase is a reverse transcriptase. In an embodiment, the RNA template includes a 5′ cap. In an embodiment, the RNA template is encapsulated in a liposome. In an embodiment, the RNA template is targeted to cells using ligands selective for T-cells, macrophages, and monocytes. In an embodiment, the T-cells are memory T-cells. In an embodiment, the memory T cells harbor active or latent HIV infection. In an embodiment, the RNA template is targeted to cells using a ligand selective for at least one specific organ system from the group consisting of liver, kidneys, lungs, liver, spleen, heart and blood vessels, GI tract, blood, bone marrow, lymphatic organs, endocrine organs, brain, spinal cord, genitourinary system and central nervous system. In an embodiment, the endocrine organs are at least one selected from the group consisting of adrenal, thyroid, and pituitary. In an embodiment, the targeted cells harbor active or latent HIV infection. In an embodiment, the RNA template includes DNA components to create chimeric templates. In an embodiment, the RNA template encodes at least one from the group consisting of a Zinc finger nuclease (ZFN), a Transcription Activator-Like Effector Nuclease (TALEN), and a gene editing enzyme. In an embodiment, the gene editing enzyme is at least one selected from the group consisting of a Cas 9 enzyme and a Cpf1 enzyme. In an embodiment, the RNA template encodes at least one selected from the group consisting of a peptide, a protein, and an enzyme. In an embodiment, the RNA template encodes a vaccine. In an embodiment, the vaccine is an immunogenic peptide or protein. In an embodiment, the peptide or protein is from a virus selected from the group consisting of influenza, VZV (chicken pox or zoster), Herpes Simplex Virus (HSV), (Human Immunodeficiency Virus (HIV), Hepatitis B Virus (HBV), Hepatitis C Virus (HCV), Measles, Mumps Rubella, Cytomegalovirus (CMV), Poliovirus, Epstein Barr Virus (EBV), Rotavirus, and bacterial immunogens. In an embodiment, the bacterial immunogens are at least one selected from the group consisting of streptococcus, clostridia, and neisseria. In an embodiment, the RNA template is administered with an RNase inhibitor. In an embodiment, at least one of a 5′ end and 3′ end of the RNA template is chemically modified to render the RNA template more resistant to exonuclease degradation. In an embodiment, the nucleic acid components are chemically modified to render the RNA template more resistant to endonuclease degradation. In an embodiment, the RNA template is generated from the plasmid pAFTAB. In an embodiment, the promoter is any eukaryotic promoter from the group consisting of EF1, CMV, EF1a, SV40, human PGK1, mouse PGK1, Ubc, human beta actin, CAG, TRE, UAS, Ac5, Polyhedrin, CaMKIIa, GAL1,10, TEF1, GDS, ADH1, CaMV35S, Ubi, H1, and U6. In an embodiment, the gene sequence is from the group consisting of EGFP, Cas9, VZV IE62, and Influenza Nucleoprotein. In an embodiment, the primer binding sequence is defined within the sequence of the RNA template. In an embodiment, a polyadenylation signal wherein the mRNA transcripts generated downstream from the double stranded DNA include a poly A tail. In an embodiment, the substance is selected from the group consisting of peptides, proteins, enzymes, antibodies, immunologically relevant proteins or peptide, short fragment RNA, short fragment DNA, ribozymes, and gene-editing enzymes.

An embodiment of the disclosure is a method for diagnosing a disorder in a mammal comprising administering to the mammal a composition comprising (a) an aviral RNA template comprising: (i) a primer binding sequence at the 3′ end of the RNA template; (ii) a sequence encoding a gene; (iii) a sequence encoding a promoter that is capable of regulating expression of the gene; wherein the aviral RNA template serves as a template for the synthesis of a complementary single stranded DNA by reverse transcriptase in cells expressing reverse transcriptase; wherein the single stranded DNA serves as a template for synthesis of a second complementary strand of DNA either by a DNA polymerase; thus resulting in a double stranded DNA; wherein the complementary single stranded DNA is not synthesized from the aviral RNA template in cells lacking reverse transcriptase; and (b) a first primer complementary to the RNA template; wherein the administration of the RNA template is through an aviral delivery technique; wherein the mammal comprises at least one cell expressing reverse transcriptase; and wherein the presence of a substance encoded by the RNA template indicates the disorder. In an embodiment, the DNA polymerase is reverse transcriptase. In an embodiment, the RNA template lacks a poly A tail. In an embodiment, the RNA template has a length of at least three hundred bases. In an embodiment, the RNA template includes no more than five viral genes. In an embodiment, the RNA template is annealed to the primer prior to administration to the mammal. In an embodiment, the primer is not annealed to the RNA template. In an embodiment, a second primer either produced in the cell or administered to the mammal primes the synthesis of the second strand of DNA either by reverse transcriptase or other DNA polymerase. In an embodiment, the RNA template encodes a polyadenylation signal. In an embodiment, the RNA template is reverse transcribed to yield a single strand DNA and then the single strand DNA is further transcribed by a DNA polymerase to yield a double strand DNA. In an embodiment, the DNA polymerase is a reverse transcriptase. In an embodiment, the RNA template includes a 5′ cap. In an embodiment, the RNA template is encapsulated in a liposome. In an embodiment, the RNA template is targeted to cells using ligands selective for T-cells, macrophages, and monocytes. In an embodiment, the t-cells are memory t-cells. In an embodiment, the memory T cells harbor active or latent HIV infection. In an embodiment, the RNA template is targeted to cells using a ligand selective for at least one specific organ system from the group consisting of liver, kidneys, lungs, liver, spleen, heart and blood vessels, GI tract, blood, bone marrow, lymphatic organs, endocrine organs, brain, spinal cord, genitourinary system and central nervous system. In an embodiment, the endocrine organs are at least one selected from the group consisting of adrenal, thyroid, and pituitary. In an embodiment, the targeted cells harbor active or latent HIV infection. In an embodiment, the RNA template includes DNA components to create chimeric templates. In an embodiment, the RNA template encodes at least one from the group consisting of a Zinc finger nuclease (ZFN), a Transcription Activator-Like Effector Nuclease (TALEN), and a gene editing enzyme. In an embodiment, the gene editing enzyme is at least one selected from the group consisting of a Cas 9 enzyme and a Cpf1 enzyme. In an embodiment, the RNA template encodes at least one selected from the group consisting of a peptide, a protein, and an enzyme. In an embodiment, the RNA template encodes a vaccine. In an embodiment, the vaccine is an immunogenic peptide or protein. In an embodiment, the peptide or protein is from a virus selected from the group consisting of influenza, VZV (chicken pox or zoster), Herpes Simplex Virus (HSV), (Human Immunodeficiency Virus (HIV), Hepatitis B Virus (HBV), Hepatitis C Virus (HCV), Measles, Mumps Rubella, Cytomegalovirus (CMV), Poliovirus, Epstein Barr Virus (EBV), Rotavirus, and bacterial immunogens. In an embodiment, the bacterial immunogens are at least one selected from the group consisting of streptococcus, clostridia, and neisseria. In an embodiment, the RNA template is administered with an RNase inhibitor. In an embodiment, at least one of a 5′ end and 3′ end of the RNA template is chemically modified to render the RNA template more resistant to exonuclease degradation. In an embodiment, the nucleic acid components are chemically modified to render the RNA template more resistant to endonuclease degradation. In an embodiment, the RNA template is generated from a plasmid inside the cell expressing reverse transcriptase. In an embodiment, the reverse transcriptase gene is delivered to the cell with the plasmid. In an embodiment, RT enzyme or mRNA is co-delivered to the cell. In an embodiment, an n base RNA is bound to a less than n base DNA, hybridized, and delivered to cell with a forward primer.

An embodiment of the disclosure is a method for preventing a disorder in a mammal comprising administering to the mammal a composition comprising (a) an aviral RNA template comprising: (i) a primer binding sequence at the 3′ end of the RNA template; (ii) a sequence encoding a gene; (iii) a sequence encoding a promoter that is capable of regulating expression of the gene; wherein the aviral RNA template serves as a template for the synthesis of a complementary single stranded DNA by reverse transcriptase in cells expressing reverse transcriptase; wherein the single stranded DNA serves as a template for synthesis of a second complementary strand of DNA either by a DNA polymerase; thus resulting in a double stranded DNA; wherein the complementary single stranded DNA is not synthesized from the aviral RNA template in cells lacking reverse transcriptase; and (b) a first primer complementary to the RNA template; wherein the administration of the RNA template is through an aviral delivery technique; wherein the mammal comprises at least one cell expressing reverse transcriptase; and wherein the presence of a substance encoded by the RNA template prevents the disorder.

In an embodiment, the DNA polymerase is reverse transcriptase. In an embodiment, the RNA template lacks a poly A tail. In an embodiment, the RNA template has a length of at least three hundred bases. In an embodiment, the RNA template includes no more than five viral genes. In an embodiment, the RNA template is annealed to the primer prior to administration to the mammal. In an embodiment, in the primer is not annealed to the RNA template. In an embodiment, a second primer either produced in the cell or administered to the mammal primes the synthesis of the second strand of DNA either by reverse transcriptase or other DNA polymerase. In an embodiment, the RNA template encodes a polyadenylation signal. In an embodiment, the RNA template is reverse transcribed to yield a single strand DNA and then the single strand DNA is further transcribed by a DNA polymerase to yield a double strand DNA. In an embodiment, the DNA polymerase is a reverse transcriptase. In an embodiment, the RNA template includes a 5′ cap. In an embodiment, the RNA template is encapsulated in a liposome. In an embodiment, the RNA template is targeted to cells using ligands selective for T-cells, macrophages, and monocytes. In an embodiment, the T-cells are memory t-cells. In an embodiment, the memory T cells harbor active or latent HIV infection. In an embodiment, the RNA template is targeted to cells using a ligand selective for at least one specific organ system from the group consisting of liver, kidneys, lungs, liver, spleen, heart and blood vessels, GI tract, blood, bone marrow, lymphatic organs, endocrine organs, brain, spinal cord, genitourinary system and central nervous system. In an embodiment, the endocrine organs are at least one selected from the group consisting of adrenal, thyroid, and pituitary. In an embodiment, in the targeted cells harbor active or latent HIV infection. In an embodiment, the RNA template includes DNA components to create chimeric templates. In an embodiment, the RNA template encodes at least one from the group consisting of a Zinc finger nuclease (ZFN), a Transcription Activator-Like Effector Nuclease (TALEN), and a gene editing enzyme. In an embodiment, the gene editing enzyme is at least one selected from the group consisting of a Cas 9 enzyme and a Cpf1 enzyme. In an embodiment, the RNA template encodes at least one selected from the group consisting of a peptide, a protein, and an enzyme. In an embodiment, the RNA template encodes a vaccine. In an embodiment, the vaccine is an immunogenic peptide or protein. In an embodiment, the peptide or protein is from a virus selected from the group consisting of influenza, VZV (chicken pox or zoster), Herpes Simplex Virus (HSV), Human Immunodeficiency Virus (HIV), Hepatitis B Virus (HBV), Hepatitis C Virus (HCV), Measles, Mumps Rubella, Cytomegalovirus (CMV), Poliovirus, Epstein Barr Virus (EBV), Rotavirus, and bacterial immunogens. In an embodiment, the bacterial immunogens are at least one selected from the group consisting of streptococcus, clostridia, and neisseria. In an embodiment, rein the RNA template is administered with an RNase inhibitor. In an embodiment, at least one of a 5′ end and 3′ end of the RNA template is chemically modified to render the RNA template more resistant to exonuclease degradation. In an embodiment, he nucleic acid components are chemically modified to render the RNA template more resistant to endonuclease degradation. In an embodiment, the condition is prevented by delivery of the composition to a cell population prone to infection. In an embodiment, the cell population is CD4 T-cells.

An embodiment of the disclosure is a method for administering in vivo a RNA template to a mammal having at least one cell expressing a reverse transcriptase enzyme, comprising administering to the mammal a composition comprising (a) an aviral RNA template comprising: (i) a primer binding sequence at the 3′ end of the RNA template; (ii) a gene sequence encoding a substance with a therapeutic or a diagnostic effect; (iii) a sequence encoding a promoter capable of regulating expression of the gene sequence; wherein the aviral RNA template serves as a template for the synthesis of a complementary single stranded DNA by a reverse transcriptase in cells expressing reverse transcriptase; wherein the single stranded DNA serves as a template for synthesis of a second complementary strand of DNA by a DNA polymerase to generate a double stranded DNA; wherein the complementary single stranded DNA is not synthesized from the aviral RNA template in cells lacking reverse transcriptase; and (b) a first primer complementary to the RNA template; wherein the administration of the RNA template is through an aviral delivery technique. In an embodiment, the DNA polymerase is a reverse transcriptase.

An embodiment of the disclosure is a method for administering in vivo a RNA template to a mammal having at least one cell expressing a reverse transcriptase enzyme, comprising administering to the mammal a composition comprising (a) an aviral RNA template comprising: (i) a primer binding sequence at the 3′ end of the RNA template; (ii) a gene sequence encoding a substance with a therapeutic or a diagnostic effect; (iii) a sequence encoding a promoter capable of regulating expression of the gene sequence; wherein the aviral RNA template serves as a template for the synthesis of a complementary single stranded DNA by a reverse transcriptase in cells expressing reverse transcriptase; wherein the single stranded DNA serves as a template for synthesis of a second complementary strand of DNA by a DNA polymerase to generate a double stranded DNA; wherein the complementary single stranded DNA is not synthesized from the aviral RNA template in cells lacking reverse transcriptase; and (b) a first primer complementary to the RNA template; wherein the administration of the RNA template is through an aviral delivery technique; and wherein the substance encoded by the gene sequence exhibits the therapeutic or the diagnostic effect. In an embodiment, the DNA polymerase is a reverse transcriptase.

An embodiment of the disclosure is a method for administering an RNA template to a mammal having at least one cell expressing a reverse transcriptase enzyme an aviral reverse transcriptase dependent (RTD) RNA template comprising (i) a primer binding sequence for reverse transcriptase mediated synthesis, wherein the primer binding sequence is at the 3′ end of the RNA template; (ii) a gene sequence encoding a protein with a therapeutic or diagnostic effect; (iii) a sequence encoding a promoter that is capable of regulating expression of the gene; wherein the RNA template is converted into DNA in cells expressing reverse transcriptase; wherein the DNA is not synthesized from the aviral RNA template in cells lacking reverse transcriptase; and wherein the cells expressing the reverse transcriptase enzyme include a primer complementary to the RNA template; and wherein the administration is through an aviral delivery technique.

An embodiment of the disclosure is an aviral reverse transcriptase dependent (RTD) RNA template comprising: (a) a primer binding sequence for reverse transcriptase mediated synthesis, wherein the primer binding sequence is at the 3′ end of the RNA template; (b) a gene sequence encoding a protein with a therapeutic or a diagnostic effect; (c) a sequence encoding a promoter that is capable of regulating expression of the gene; wherein the RNA template is converted into DNA in cells expressing reverse transcriptase; and wherein the DNA is not synthesized from the aviral RNA template in cells lacking reverse transcriptase. In an embodiment, the RNA template is present on a plasmid.

An embodiment of the disclosure is a RNA-based composition comprising: (a) an aviral reverse transcriptase dependent (RTD) RNA template comprising (i) primer binding sequence for reverse transcriptase mediated synthesis, the primer binding sequence being at the 3′ end of the RNA template; (ii) a sequence encoding a gene for therapeutic or diagnostic effects; (iii) a sequence encoding a promoter that is capable of regulating expression of the protein; wherein the RNA template is converted into DNA in cells expressing reverse transcriptase; wherein the DNA is not synthesized from the aviral RNA template in cells lacking reverse transcriptase; and (b) a delivery system for the RNA template. In an embodiment, the RNA template is present on a plasmid. In an embodiment, the composition is therapeutic. In an embodiment, the composition is diagnostic. In an embodiment, the delivery system is selected from the group consisting of a liposome encapsulating the RNA template, a virus-like polymer conjugated directly to the RNA template, a lipid conjugated directly to the RNA template, a polylysine-containing molecule electrostatically conjugated to the RNA template, and a polymer capable of binding and delivering RNA sequences of greater than 300 bases into cells. In an embodiment, the polymer is polyethyleneimine (PEI). In an embodiment, the composition further comprises a ligand conjugated to the delivery system, wherein the ligand targets cells to which the RNA template is to be delivered. In an embodiment, the delivery system comprises a ligand conjugated directly to the RNA template.

An embodiment of the disclosure is a method of administering an RNA template in vitro to at least one cell line expressing a reverse transcriptase enzyme comprising: (a) an aviral RNA template comprising: (i) a primer binding sequence at the 3′ end of the RNA template; (ii) a gene sequence encoding protein having a therapeutic or a diagnostic effect; (iii) a sequence encoding a promoter capable of regulating expression of the gene; wherein the RNA template serves as a template for the synthesis of a complementary single stranded DNA by reverse transcriptase in cells expressing reverse transcriptase; wherein the DNA is not synthesized from the aviral RNA template in cells lacking reverse transcriptase; and wherein the single stranded DNA serves as a template for synthesis of a second complementary strand of DNA either by a DNA polymerase; generating a double stranded DNA; wherein the administration of the RNA template is through an aviral delivery technique; and (b) a first primer complementary to the RNA template. In an embodiment, the DNA polymerase is reverse transcriptase. In an embodiment, the at least one cell line expressing a reverse transcriptase enzyme is a permanent GL261 cell line constitutively producing reverse transcriptase. In an embodiment, the permanent GL261 cell line is GL261-RT786.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 depicts Agilent Bioanalyzer results from RNA degradation assays on eGFP RNA (Unmodified).

FIG. 2 depicts Agilent Bioanalyzer results of the eGFP RNA (Alpha-Thio-A) degradation assays.

FIG. 3 depicts Agilent Bioanalyzer results of the eGFP RNA (Alpha-Thio-U) degradation assays.

FIG. 4 depicts Agilent Bioanalyzer results of the eGFP RNA (5-Me-C and Pseudo-U) degradation assays.

FIG. 5 depicts Agilent Bioanalyzer results of RNase T1 degradation assays.

FIG. 6 depicts Agilent Bioanalyzer results of the cDNA synthesis assays.

FIG. 7 depicts Agilent Bioanalyzer results of the HIV RT degradation assays.

FIG. 8 depicts Agilent Bioanalyzer results of HIV RT degradation assays.

FIG. 9 depicts a design schematic of eGFP RNA templates indicating placement of primers and expected amplicon sizes.

FIG. 10 depicts Agilent Bioanalyzer results of cDNA synthesis reactions.

FIG. 11 depicts agarose gel electrophoresis of end point PCR reactions.

FIG. 12 depicts agarose gel electrophoresis results from the first iteration of eGFP RNA (Unmodified) DNase treatment.

FIG. 13 depicts agarose gel electrophoresis results from the second iteration of eGFP RNA (Unmodified) DNase treatment.

FIG. 14 depicts agarose gel electrophoresis results from the third iteration of eGFP RNA (Unmodified) DNase treatment.

FIG. 15 depicts agarose gel electrophoresis results from the fourth iteration of eGFP RNA (Unmodified) DNase treatment.

FIG. 16 depicts agarose gel electrophoresis results from the third iteration of all four RNA templates DNase treatment.

FIG. 17 depicts agarose gel electrophoresis results from the fourth iteration of DNase treatment.

FIG. 18 depicts Agilent Bioanalyzer results of cDNA synthesis reactions of DNase free RNA templates.

FIG. 19 depicts the agarose gel electrophoresis results of end point PCR assays on cDNA reactions.

FIG. 20A-20C depict the sequence for gene synthesis.

FIG. 21A-21G depicts the pAFTAB in pUC57 AMP plasmid.

FIG. 22A-22C depicts the generated sequence.

FIG. 23 depicts a diagram of the pAFTAB plasmid.

FIG. 24 depicts a chart displaying obtaining a desired mRNA from the RNA template.

FIG. 25 depicts a gel of the results of the PCR from the RT reaction using WT RNA and Superscript Enzyme.

FIG. 26 depicts a gel of the results of the PCR from the no-RT control reaction using Superscript Enzyme.

FIG. 27 depicts a gel of the results of the PCR from the RT reactions using Superscript Enzyme and SV40 DNA primer.

FIG. 28 depicts a gel of the results of the PCR from the RT reaction using WT RNA and Superscript Enzyme.

FIG. 29 depicts a gel of the results of the PCR from the RT reaction using Superscript enzyme and SV40 RNA primer.

FIG. 30 depicts a gel of the results of the PCR from the RT reaction using HIV RT and SV40 DNA and RNA primers.

FIG. 31 depicts a gel of the results from the PCR from the RT reaction using HIV RT and SV40 DNA and RNA primers plus β-Thujaplicinol.

FIG. 32A-32C depict gels of the results of the PCR from the RT reaction using HIV RT, SV40 DNA, and RNA primers plus β-Thujaplicinol in designated reactions.

FIG. 33A-33C depict gels of the results of the PCR from RT reaction using HIV RT and Phosphorothioate Primer DNA plus β-Thujaplicinol in designated reactions.

FIG. 34 depicts the WT eGFP Consensus sequence.

FIG. 35 depicts the Alpha-Thio-Uridine eGFP Consensus sequence.

FIG. 36 depicts p51/p66 dimerization: whole cell extracts staining from Native PAGE.

FIG. 37 depicts p51/p66 dimerization: anti p51/p66 Western blotting from Native PAGE.

FIG. 38 depicts a gel of PCR before and after DNase treatment.

FIG. 39 depicts a gel of mRNA quality check before and after DNase treatment.

FIG. 40 depicts reverse transcriptase activity of cell extracts. Average CPMs were plotted as a function of extract volume included in the reaction.

FIG. 41 depicts reverse transcriptase activity of cell extracts. Average CPMs for each reaction conditions are presented in bar graph format. Error bars represent standard errors of the mean.

FIG. 42 is a second embodiment of an RNA template of the present invention.

FIG. 43 is a third embodiment of an RNA template of the present invention.

FIG. 44 is a fourth embodiment of an RNA template of the present invention.

FIG. 45 is a generalized representation of a poly A signal sequence.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

As used herein, the term “U5 sequence” means and refers to the repeated sequence at the 5′ end of a retroviral RNA.

As used herein, the term “U3 sequence” means and refers to the repeated sequence at the 3′ end of a retroviral RNA.

As used herein, the term “R sequence” means and refers to a sequence that is repeated at the ends of a retroviral RNA.

As used herein, the term “virus-like polymer” means and refers to a polymer based transfection reagent able to mimic the viral infection process by an active endosome escape mechanism. Viromer® is a product of lipocalyx.

A reverse transcriptase is an enzyme utilized by retroviruses to convert negative strand viral RNA into DNA. The same enzyme then synthesizes the complementary strand of the DNA yielding a double stranded DNA derived from the viral genome. This material is then inserted into the host DNA. Other than telomerases, reverse transcriptase is not expressed in uninfected human cells.

Some key viral pathogens that affect humans and rely on reverse transcriptase include HIV and Human T Cell Leukemia Virus. Mouse viruses such as MMLV also rely on reverse transcriptase for infection. Although recombinant viruses have been used to infect cells and express genes, aviral delivery of a custom RNA template progene to infected cells expressing reverse transcriptase has not been reported. RNA is unstable under physiological conditions and stabilizing modifications of RNA may render the template unreadable by reverse transcriptase. The RNA template needs stabilizing modifications which do not alter template function. The RNA template can be packaged in nanoparticles or liposomes for efficient delivery to target cells. Furthermore, a custom RNA should encode a promoter, gene and possibly a polyadenylation signal such that upon reverse transcription into first strand of DNA and subsequent synthesis of the second DNA strand, the double strand encodes a full gene under a functional promoter.

Mammalian cells do not express reverse transcriptase (with the exception of telomerase). Therefore, a synthetic RNA template can be designed such that it serves as a progene expressed only in infected cells, i.e., where reverse transcriptase is present. The RNA template is designed such that “primers” naturally found in cells, such as tRNA, can prime the RT reaction both in the synthesis of the first and second strand. In an embodiment, the RNA template can be designed such that only custom primers (RNA or DNA) can prime the two-step reaction of converting the RNA template in to double stranded DNA gene. The gene ultimately expressed would be operational under a functional promoter (encoded in the synthetic template) and encode including but not limited to peptides (such as MHC peptides or enzyme inhibitors), proteins, enzymes, antibodies, immunologically relevant proteins or peptide, short fragment RNA or DNA (such as antisense or siRNA), ribozymes, gene-editing enzymes including but not limited to CRISP-R. In an embodiment, the promoter is a eukaryotic promoter from the group consisting of EF1, CMV, EF1a, SV40, human PGK1, mouse PGK1, Ubc, human beta actin, CAG, TRE, UAS, Ac5, Polyhedrin, CaMKIIa, GAL1,10, TEF1, GDS, ADH1, CaMV35S, Ubi, H1, and U6. In an embodiment, any known eukaryotic promoter that can be utilized. The RNA template can be a natural template with wild type RNA or be modified with analogs of RNA. In an embodiment, the modifications can include but are not limited to phosphorothioate, 2-thiouridine, 5mC, pseudouridine, 2-Amino. In an embodiment, any known modification can be utilized. The RNA or DNA primer can be separately delivered or concomitantly delivered with the custom RNA template. The RNA or DNA primers can be comprised of natural bases or modified with including but not limited to phosphorothioate, 2-thiouridine, 5mC, pseudouridine, and 2-Amino. The RNA or DNA primer can be approximately 20 bases or significantly longer as it forms a hybrid structure with the RNA template.

In an embodiment, the function of RT is utilized to convert synthetic RNA templates into double stranded DNA for a therapeutic function, to aid in diagnosis (such as assessing extent of infection), and in prevention of a condition. Such therapy may also be useful in clearing infection in latent cell populations such as memory T-cells. Memory T-cells are often the last bastion of infection in retroviral diseases such as HIV. These RNA template constructs may be useful in preventing infection in exposed individuals by delivery of the RNA templates/primers to cell populations prone to infection such as CD4 T-cells.

In an embodiment, the RNA template does not comprise an R, U5, or U3 sequence. In an embodiment, the RNA template does comprise an R, U5, or U3 sequence.

In an embodiment, the reverse transcriptase is selected from the group consisting of HIV RT, HTLV RT, EBOLA RT, Hep C RT, and MMLV RT. In an embodiment, any known reverse transcriptase can be utilized.

In an embodiment, certain RNA templates may be more efficiently reverse transcribed into DNA in the presence of RNase H inhibitors such as beta thujaplicinol or other RNase H inhibitors. In an embodiment, any known RNase H inhibitors can be utilized.

In an embodiment, RNA templates greater than 200 bases can be reverse transcribed with HIV RT and amplified with PCR. The templates can be more efficiently transcribed in the presence of an RNase H inhibitor such as beta thujaplicinol. The double stranded DNA resulting from the PCR can be a double stranded gene product.

In an embodiment, the RNA templates and relevant primers can be delivered in liposomes or nanoparticles. In an embodiment, the primer is not delivered with the RNA template. In an embodiment, the primer is not delivered separately from the RNA template. The liposomes and nanoparticles can be functionalized and targeted to relevant cell populations with targeting moieties including but not limited to peptides, antibodies, and aptamers.

In an embodiment, the RNA templates and primers can be delivered to cells in combination with a gene or messenger RNA encoding a reverse transcriptase. In an embodiment, the expression of both the reverse transcriptase and the RNA template and primer would be required for expression of the information encoded by the RNA template. In an embodiment, this is similar to other enzyme and pro-drug combinations such thymidine kinase and acyclovir. In an embodiment, the primer is not delivered with the RNA template. In an embodiment, the primer is not delivered separately from the RNA template.

In vitro and in vivo reverse transcriptase-dependent aviral retrogene therapy with custom RNA template/primers (WT and modified)-novel selective gene therapy approach for cells infected with retrovirus is disclosed herein.

### Plasmid

In an embodiment, the gene therapy approach includes a plasmid to produce a template in vitro under control of a promoter including but not limited to HIV LTR and TAT, a gene cassette, and a polyadenylation signal. Retrogenes for expression of VZV, NP, Influenza, Chicken Pox, Measles, Mumps, Rubella, DPT, Polio, Bacterial Proteins for Immunization, CCR5, IE62, IE63, Influenza, Radiosensitizer, in addition to other genes including an integration gene can be present on the plasmid. The plasmid can be used to produce RNA template and primers in vivo.

### Liposomal Delivery

In an embodiment, the RNA template can be delivered via liposome. An anti-CD2 antibody with site mutations in the amino acid sequence for coupling to liposomes can be used for RNA delivery to latent cells. Delivery can occur in the presence and absence of RNaseH inhibitors such as thujaplicinol. Reverse transcriptases including but not limited to HIV RT EBOLA RT, Hep C RT, and HTLV RT can be utilized.

RNA Template and/or Primer

The RNA template and/or primer (RNA or DNA primer) can be modified with chemical or enzymatic modifications including phosphorothioate, 2Amino, 2thiouridine, pseudouridine, 2′F or 5mC or any combination thereof. The RNA template or primer can also be modified with 5′ or 3′ modifications. In an embodiment, the RNA templates can be with or without a poly A tail.

In an embodiment, short (<150 bases) RNA templates with or without primers (modified and WT) are delivered for reverse transcription in RT positive cells (RNA prodrug) in the presence and absence of RNASE H inhibitors.

In an embodiment, RT-PCR with HIV Reverse Transcriptase using Wild Type and Modified RNA Templates and Primers in the Presence and Absence of RnaseH Inhibitors is performed. In an embodiment, the RT-PCR is performed on RNA templates>150 bases, with modified RNA templates, with templates with and without a poly A tail, in the presence and absence of Beta Thujaplicinol and other RNase H inhibitors, as a diagnostic for retrovirus infection, and for amplification of a naked gene.

In an embodiment, a co-transfection with a reverse transcriptase and a RNA template gene therapy model similar to HSV thymidine kinase and acyclovir can utilized.

In an embodiment, telomerase dependent expression of RNA template can be performed.

In an embodiment, the primer can be RNA or DNA.

In an embodiment, the RNA template or primer can be WT or modified.

In an embodiment, the dataset can include Superscript and HIV RT. In an embodiment, the template can be 900 bases, there can be 4 RNA templates, and Superscript and HIV RT can be used as the reverse transcriptase. In an embodiment, the plasmid is pAftab, the RNA template is 2000 bases, there can be 4 RNAs template, the primers can be RNA and/or DNA, the primers and/or template can be modified, and the experiment can be done in the presence or absence of an RNase H inhibitor.

In an embodiment, an approximately 150 base RNA can be reverse transcribed and amplified by RT-PCR.

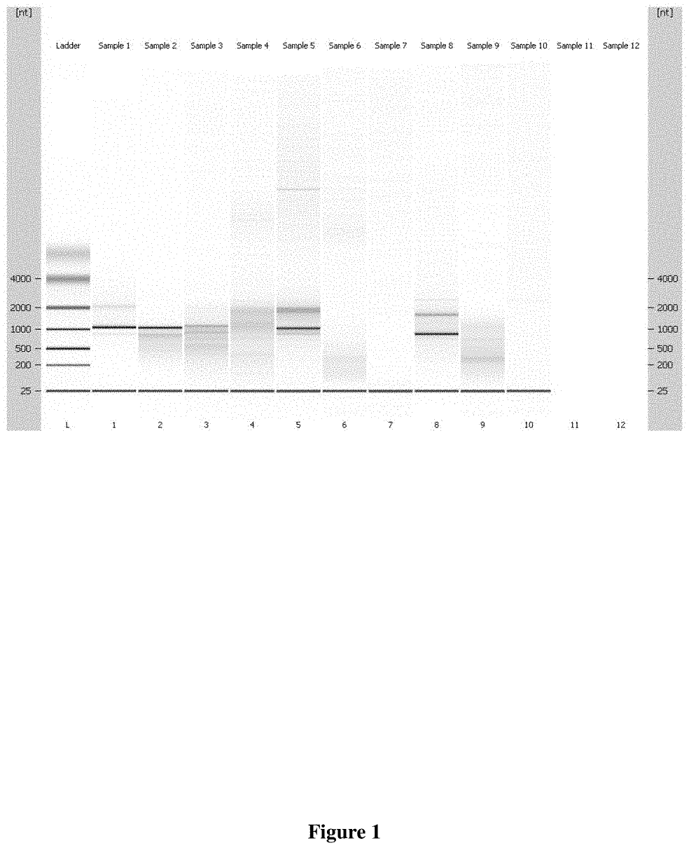

A 1 kb mRNA with a poly A tail template was amplified by RT-PCR with primers. The experiment used 1) Superscript as the enzyme and the following RNA: WT, 5mC/P, phosphorothioate, and 2-thiouridine and 2) HIV RT as the enzyme and the following RNA: WT, 5mC/P, phosphorothioate, and 2-thiouridine. FIGS. 1-19.

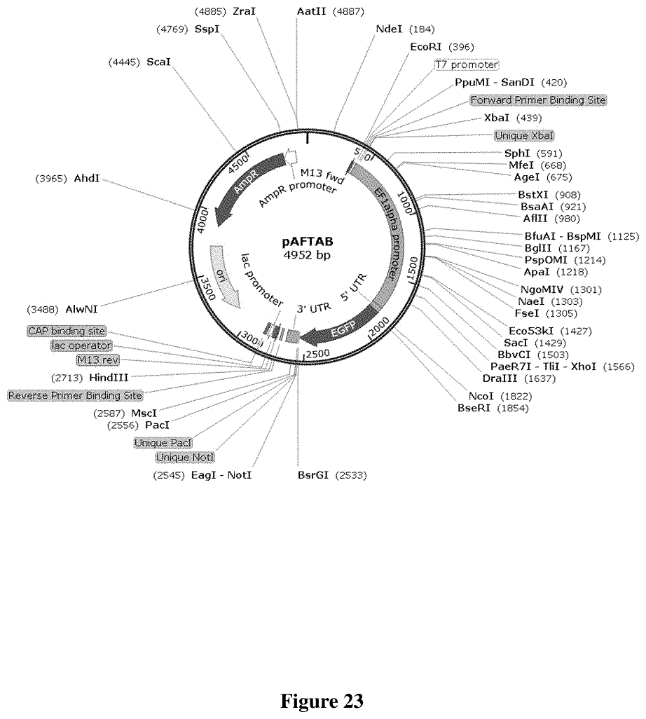

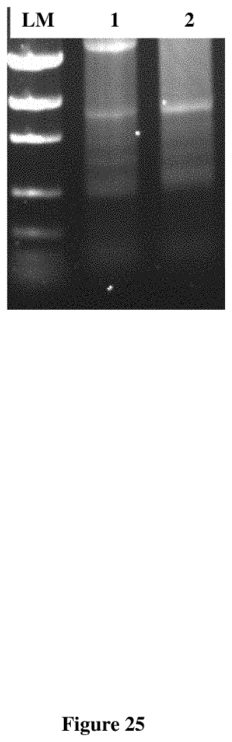

A 2.2 kb RNA template was amplified by RT-PCR. RNA templates were synthesized with plasmid pAFTAB. FIG. 23. The experiment used 1) Superscript as the enzyme and the following RNA: WT, 5mC/P, phosphorothioate, and 2-thiouridine and 2) HIV RT as the enzyme and the following RNA: WT, 5mC/P, phosphorothioate, and 2-thiouridine. FIGS. 25-33. The experiment was performed in the presence of primers, a phosphorothioate primer, a-RNA and DNA primer, and/or an RnaseH Inhibitor.

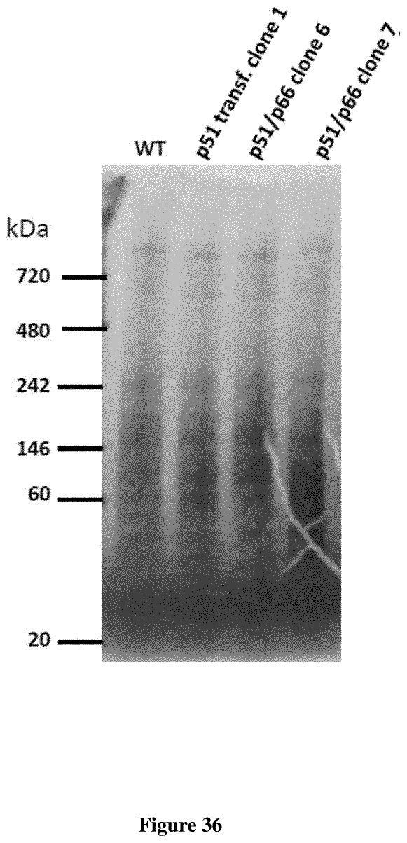

In an embodiment, an HIV RT-Transfected Cell Line using GL261 cells was created. Example 9. FIGS. 36-39. The RNA is a 2 kb RNA template that is WT, 5mC/P, phosphorothioate, or 2-thiouridine. The experiment is performed in the presence of primers, a phosphorothioate primer, a RNA and DNA primer, and/or an RnaseH Inhibitor.

### EXAMPLES

### Example 1

### RNA Species

The stock RNA species are found in Table 1.

TABLE 1
RNA species

| | | Stock | |
|---|---|---|---|
| | Volume | Concentration | Total amount |
| | Received | Received | Received |
| RNA Species | (mL) | (ng/μL) | (μg) |
| eGFP RNA Unmodified | 2.95 | 890 | 2630 |
| eGFP RNA (Alpha-Thio-A) | 0.411 | 530 | 218 |
| eGFP RNA (Alpha-Thio-U) | 2.65 | 806 | 2140 |
| eGFP RNA (5-Me—C and | 2.72 | 818 | 2225 |
| Pseudo-U) | | | |

The RNA species were received from TriLink Biotechnologies. Working concentration (125 ng/μL) and stock concentration aliquots of each RNA were prepared.

### Example 2

### RNA Degradation Assays

The experiment was performed to evaluate the sensitivity of the four RNA templates in Table 1 to various RNase enzyme species. RNA degradation assays were carried out to evaluate the degradation effects of three enzymes: HIV-RT (Worthington Biochemicals), RNase H (New England Biolabs), and Exonuclease T (New England Biolabs).

An HIV-RT reaction buffer was prepared according to the manufacturer's recommendation. The buffer was made by adding 0.606 g of Tris, 2.28 mL of 1 N HCl, and 97.72 mL H₂O. The pH was titrated to 8.3 and 1 mL of 800 mM MgCl₂ was added.

eGFP RNA (Unmodified)

Methods: To evaluate the degradation effects of the enzymes on the eGFP RNA (Unmodified), the reactions in Table 2 were carried out following the manufacturer's recommendations for each enzyme:

TABLE 2
RNA degradation assays on eGFP RNA (Unmodified)

| | RNA Used | Reaction Buffer | Enzyme Used | Incubation |
|---|---|---|---|---|
| Sample | (Quantity) | Used (Quantity) | (Quantity) | Conditions |
| 1 | eGFP RNA | — | — | — |
| | Unmodified (1 μg) | | | |
| 2 | eGFP RNA | — | — | 37° C. 30 min, |
| | Unmodified (1 μg) | | | 70° C. 20 min |
| 3 | eGFP RNA | Prepared HIV RT | — | 37° C. 30 min, |
| | Unmodified (1 μg) | (1 μL) | | 70° C. 20 min |
| 4 | eGFP RNA | 10 X NEB Buffer | — | 37° C. 30 min, |
| | Unmodified (1 μg) | 3 (1 μL) | | 70° C. 20 min |
| 5 | eGFP RNA | 10 X NEB Buffer | — | 25° C. 30 min, |
| | Unmodified (1 μg) | 4 (1 μL) | | 65° C. 20 min |
| 6 | eGFP RNA | Prepared HIV RT | HIV RT (20 U) | 37° C. 30 min, |
| | Unmodified (1 μg) | (1 μL) | | 70° C. 20 min |
| 7 | eGFP RNA | 10 X NEB Buffer | RNase H (50 U) | 37° C. 30 min, |
| | Unmodified (1 μg) | 3 (1 μL) | | 70° C. 20 min |
| 8 | eGFP RNA | 10 X NEB Buffer | Exonuclease T | 25° C. 30 min, |
| | Unmodified (1 μg) | 4 (1 μL) | (5 U) | 65° C. 20 min |
| 9 | eGFP RNA | 10 X NEB Buffer | HIV RT (20 U) | 37° C. 30 min, |
| | Unmodified (1 μg) | 3 (1 μL) | | 70° C. 20 min |

Results: The reactions from Table 2 were run on an Agilent Bioanalyzer 2100 RNA 6000 Nano Chip. The results are displayed in FIG. 1. The results showed that the eGFP RNA (Unmodified) received from TriLink Biotechnologies was stable and full length. Heavy degradation was observed when that the eGFP RNA (Unmodified) was incubated with either HIV RT or RNase H enzymes. The Exonuclease T enzyme seemed unable to degrade the RNA. The assay also revealed that the HIV RT Reaction buffer and NEB buffer 3 caused degradation of the RNA. In an embodiment, NEB buffer 4 can be used to eliminate the endogenous RNase activity observed in the HIV RT Reaction buffer and NEB Buffer 3.

eGFP RNA (Alpha-Thio-A)

Methods: To evaluate the degradation effects of the enzymes on the eGFP RNA (Alpha-Thio-A), the reactions in Table 3 were carried out following the manufacturer's recommendations for each enzyme:

TABLE 3
RNA degradation assays on eGFP RNA (Alpha-Thio-A)

| | RNA Used | Reaction Buffer | Enzyme Used | Incubation |
|---|---|---|---|---|
| Sample | (Quantity) | Used (Quantity) | (Quantity) | Conditions |
| 1 | eGFP RNA | — | — | — |
| | (Alpha-Thio-A) | | | |
| | (1 μg) | | | |
| 2 | eGFP RNA | — | — | 37° C. 30 min, |
| | (Alpha-Thio-A) | | | 70° C. 20 min |
| | (1 μg) | | | |
| 3 | eGFP RNA | Prepared HIV RT | — | 37° C. 30 min, |
| | (Alpha-Thio-A) | (1 μL) | | 70° C. 20 min |
| | (1 μg) | | | |
| 4 | eGFP RNA | 10 X NEB Buffer 3 | — | 37° C. 30 min, |
| | (Alpha-Thio-A) | (1 μL) | | 70° C. 20 min |
| | (1 μg) | | | |
| 5 | eGFP RNA | 10 X NEB Buffer 4 | — | 25° C. 30 min, |
| | (Alpha-Thio-A) | (1 μL) | | 65° C. 20 min |
| | (1 μg) | | | |
| 6 | eGFP RNA | 10 X NEB Buffer 4 | — | 37° C. 30 min, |
| | (Alpha-Thio-A) | (1 μL) | | 70° C. 20 min |
| | (1 μg) | | | |
| 7 | eGFP RNA | Prepared HIV RT | HIV RT (20 U) | 37° C. 30 min, |
| | (Alpha-Thio-A) | (1 μL) | | 70° C. 20 min |
| | (1 μg) | | | |
| 8 | eGFP RNA | 10 X NEB Buffer 4 | RNase H (50 U) | 37° C. 30 min, |
| | (Alpha-Thio-A) | (1 μL) | | 70° C. 20 min |
| | (1 μg) | | | |
| 9 | eGFP RNA | 10 X NEB Buffer 4 | Exonuclease T | 25° C. 30 min, |
| | (Alpha-Thio-A) | (1 μL) | (5 U) | 65° C. 20 min |
| | (1 μg) | | | |
| 10 | eGFP RNA | 10 X NEB Buffer 4 | HIV RT (20 U) | 37° C. 30 min, |
| | (Alpha-Thio-A) | (1 μL) | | 70° C. 20 min |
| | (1 μg) | | | |

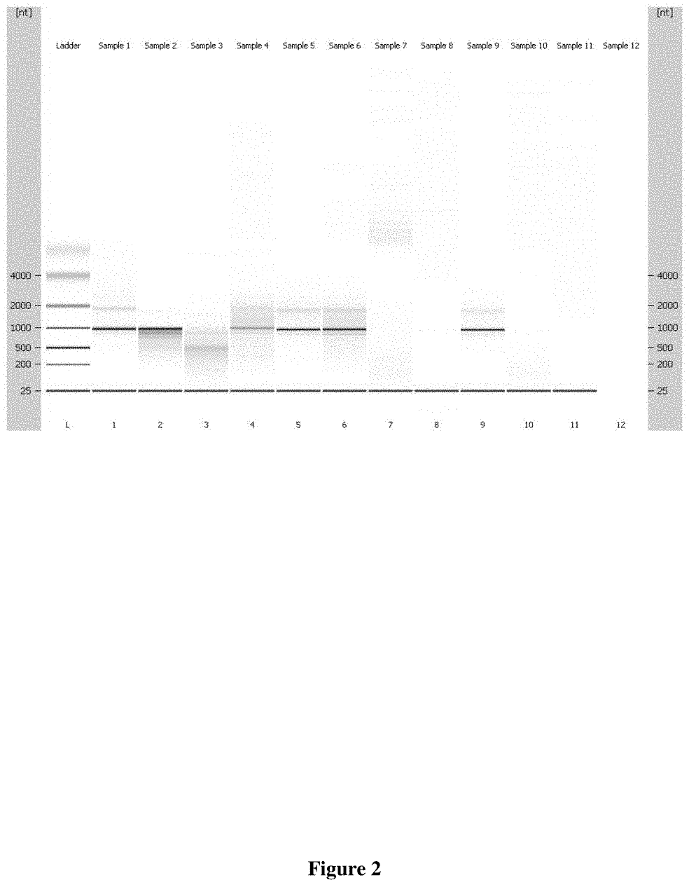

Results: The reactions from Table 3 were run on an Agilent Bioanalyzer 2100 RNA 6000 Nano Chip. The results are displayed in FIG. 2, the Agilent Bioanalyzer results of the eGFP RNA (Alpha-Thio-A) degradation assays. The results indicated that the eGFP RNA (Alpha-Thio-A) received from TriLink Biotechnologies was stable and full length. Heavy degradation was observed when the eGFP RNA (Alpha-Thio-A) was incubated with either HIV RT or RNase H enzymes. The Exonuclease T enzyme seemed unable to degrade the RNA.

eGFP RNA (Alpha-Thio-U)

Methods: To evaluate the degradation effects of the enzymes on the eGFP RNA (Alpha-Thio-U), the reactions in Table 4 were carried out following the manufacturer's recommendations for each enzyme:

TABLE 4
RNA degradation assays on eGFP RNA (Alpha-Thio-U)

| | RNA Used | Reaction Buffer | Enzyme Used | Incubation |
|---|---|---|---|---|
| Sample | (Quantity) | Used (Quantity) | (Quantity) | Conditions |
| 1 | eGFP RNA (Alpha- | — | — | — |
| | Thio-U) (1 μg) | | | |
| 2 | eGFP RNA (Alpha- | — | — | 37° C. 30 min, |
| | Thio-U) (1 μg) | | | 70° C. 20 min |
| 3 | eGFP RNA (Alpha- | 10 X NEB Buffer 4 | — | 37° C. 30 min, |
| | Thio-U) (1 μg) | (1 μL) | | 70° C. 20 min |
| 4 | eGFP RNA (Alpha- | 10 X NEB Buffer 4 | — | 25° C. 30 min, |
| | Thio-U) (1 μg) | (1 μL) | | 65° C. 20 min |
| 5 | eGFP RNA (Alpha- | 10 X NEB Buffer 4 | HIV RT (20 U) | 37° C. 30 min, |
| | Thio-U) (1 μg) | (1 μL) | | 70° C. 20 min |
| 6 | eGFP RNA (Alpha- | 10 X NEB Buffer 4 | RNase H (50 U) | 37° C. 30 min, |
| | Thio-U) (1 μg) | (1 μL) | | 70° C. 20 min |
| 7 | eGFP RNA (Alpha- | 10 X NEB Buffer 4 | Exonuclease T | 25° C. 30 min, |
| | Thio-U) (1 μg) | (1 μL) | (5 U) | 65° C. 20 min |

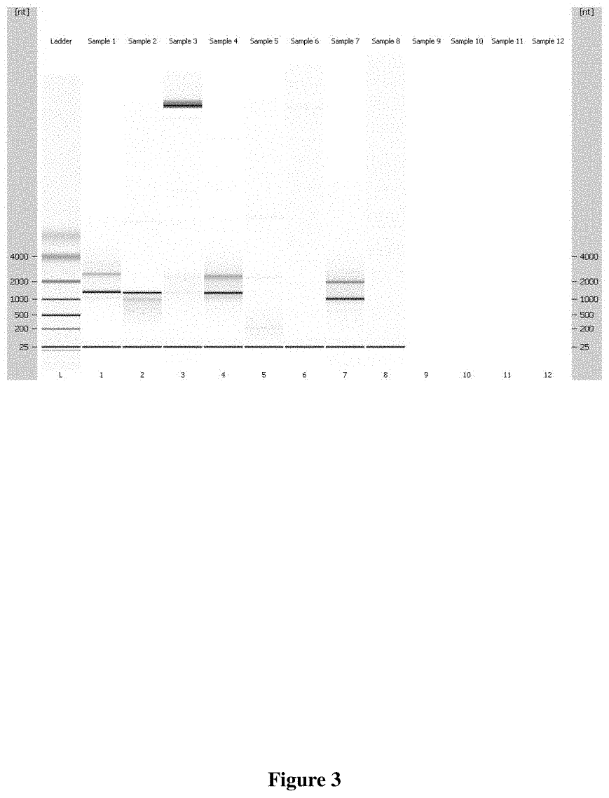

Results: The reactions from Table 4 above were then run on an Agilent Bioanalyzer 2100 RNA 6000 Nano Chip. The results are displayed in FIG. 3, Agilent Bioanalyzer results of eGFP RNA (Alpha-Thio-U) degradation assays.

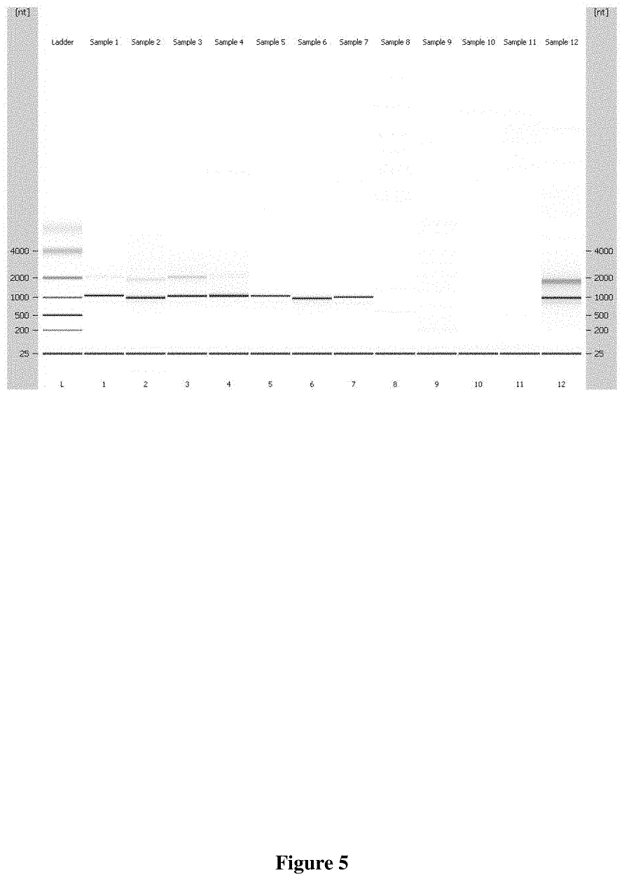

The results showed that the eGFP RNA (Alpha-Thio-U) received from TriLink Biotechnologies was stable and full length. Heavy degradation was observed when the eGFP RNA (Alpha-Thio-U) was incubated with either HIV RT or RNase H enzymes. The Exonuclease T enzyme again seemed unable to degrade the RNA. The large molecular weight band seen in sample 3 was likely an artifact of the Agilent gel matrix. This reaction was repeated as sample 12 in FIG. 5 and it was confirmed that the higher molecular weight band initially observed was an artifact introduced by the Agilent assay.

eGFP RNA (5-Me-C and Pseudo-U)

Methods: To evaluate the degradation effects of the enzymes on the eGFP RNA (5-Me-C and Pseudo-U), the reactions in Table 5 were carried out following the manufacturer's recommendations for each enzyme:

TABLE 5
RNA degradation assays on eGFP RNA (5-Me—C and Pseudo-U)

| | RNA Used | Reaction Buffer | Enzyme Used | Incubation |
|---|---|---|---|---|
| Sample | (Quantity) | Used (Quantity) | (Quantity) | Conditions |
| 1 | eGFP RNA (5-Me—C | — | — | — |
| | and Pseudo-U) (1 μg) | | | |
| 2 | eGFP RNA (5-Me—C | — | — | 37° C. 30 min, |
| | and Pseudo-U) (1 μg) | | | 70° C. 20 min |
| 3 | eGFP RNA (5-Me—C | 10 X NEB Buffer 4 | — | 37° C. 30 min, |
| | and Pseudo-U) (1 μg) | (1 μL) | | 70° C. 20 min |
| 4 | eGFP RNA (5-Me—C | 10 X NEB Buffer 4 | — | 25° C. 30 min, |
| | and Pseudo-U) (1 μg) | (1 μL) | | 65° C. 20 min |
| 5 | eGFP RNA (5-Me—C | 10 X NEB Buffer 4 | HIV RT (20 U) | 37° C. 30 min, |
| | and Pseudo-U) (1 μg) | (1 μL) | | 70° C. 20 min |
| 6 | eGFP RNA (5-Me—C | 10 X NEB Buffer 4 | RNase H (50 U) | 37° C. 30 min, |
| | and Pseudo-U) (1 μg) | (1 μL) | | 70° C. 20 min |
| 7 | eGFP RNA (5-Me—C | 10 X NEB Buffer 4 | Exonuclease T | 25° C. 30 min, |
| | and Pseudo-U) (1 μg) | (1 μL) | (5 U) | 65° C. 20 min |

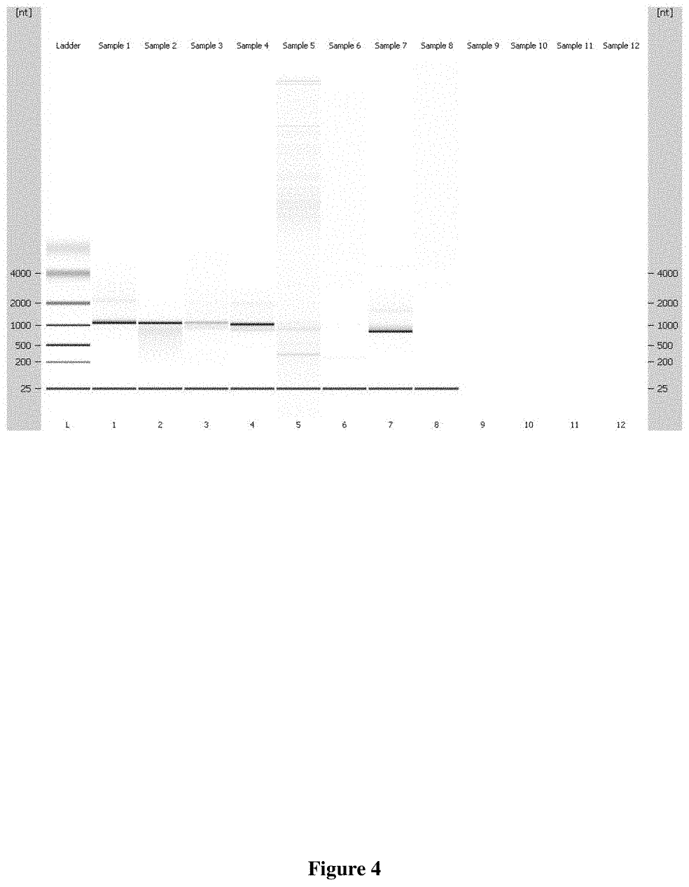

Results: The reactions from Table 5 above were run on an Agilent Bioanalyzer 2100 RNA 6000 Nano Chip. The results are displayed in FIG. 4, Agilent Bioanalyzer results of eGFP RNA (5-Me-C and Pseudo-U) degradation assays. The results showed that the eGFP RNA (5-Me-C and Pseudo-U) received from TriLink Biotechnologies was stable and full length. Heavy degradation was observed when the eGFP RNA (5-Me-C and Pseudo-U) was incubated with either HIV RT or RNase H enzymes. The Exonuclease T enzyme again seemed unable to degrade the RNA.

Upon consultation with New England Biolabs, the manufacturer of the Exonuclease T enzyme, it was determined that the particular enzyme was not capable of efficiently degrading ssRNA molecules. RNase T1 (Thermo Scientific) was chosen as a replacement enzyme and was subsequently tested against all four RNA templates in the degradation assay. Table 6 and FIG. 5. This enzyme is known to specifically degrade ssRNA molecules at guanine residues.

### RNase T1 Degradation Assay

Methods: To evaluate the degradation effects of RNase T1 enzyme on the four RNA templates, the reactions in Table 6 were carried out following the manufacturer's recommendations:

TABLE 6
RNase T1 degradation assays on four RNA templates

| | RNA Used | Reaction Buffer | Enzyme Used | Incubation |
|---|---|---|---|---|
| Sample | (Quantity) | Used (Quantity) | (Quantity) | Conditions |
| 1 | eGFP RNA | — | — | — |
| | (Unmodified) (1 μg) | | | |
| 2 | eGFP RNA (Alpha- | — | — | — |
| | Thio-A) (1 μg) | | | |
| 3 | eGFP RNA (Alpha- | — | — | — |
| | Thio-U) (1 μg) | | | |
| 4 | eGFP RNA (5-Me—C and | — | — | — |
| | Pseudo-U) (1 μg) | | | |
| 5 | eGFP RNA | RNase T1 | — | 37° C. 30 min, |
| | (Unmodified) (1 μg) | Buffer (1 μL) | | 70° C. 20 min |
| 6 | eGFP RNA (Alpha- | RNase T1 | — | 37° C. 30 min, |
| | Thio-A) (1 μg) | Buffer (1 μL) | | 70° C. 20 min |
| 7 | eGFP RNA (Alpha- | RNase T1 | — | 37° C. 30 min, |
| | Thio-U) (1 μg) | Buffer (1 μL) | | 70° C. 20 min |
| 8 | eGFP RNA | RNase T1 | RNase T1 Enzyme | 37° C. 30 min, |
| | (Unmodified) (1 μg) | Buffer (1 μL) | (10 U) | 70° C. 20 min |
| 9 | eGFP RNA (Alpha- | RNase T1 | RNase T1 Enzyme | 37° C. 30 min, |
| | Thio-A) (1 μg) | Buffer (1 μL) | (10 U) | 70° C. 20 min |
| 10 | eGFP RNA (Alpha- | RNase T1 | RNase T1 Enzyme | 37° C. 30 min, |
| | Thio-U) (1 μg) | Buffer (1 μL) | (10 U) | 70° C. 20 min |
| 11 | eGFP RNA (5-Me—C and | RNase T1 | RNase T1 Enzyme | 37° C. 30 min, |
| | Pseudo-U) (1 μg) | Buffer (1 μL) | (10 U) | 70° C. 20 min |
| 12 | eGFP RNA (Alpha- | 10X NEB | — | 37° C. 30 min, |
| | Thio-U) (1 μg) | Buffer 4 (1 μL) | | 70° C. 20 min |

Results: The reactions from Table 6 were run on an Agilent Bioanalyzer 2100 RNA 6000 Nano Chip. The results are displayed in FIG. 5, Agilent Bioanalyzer results of RNase T1 degradation assays. The results show that the RNase T1 enzyme is efficient at degrading all four of the RNA templates in this study. Sample 12 in Table 6 was a repeat of sample 3 from FIG. 3. As suspected, the higher molecular weight band initially observed was as artifact introduced by the Agilent assay.

### Example 3

### cDNA Synthesis

The purpose of experiment was to evaluate the ability of the HIV Reverse Transcriptase enzyme to generate cDNA molecules from the four RNA templates listed in Table 1. The four RNA templates were incubated with reaction buffer, dNTPs, Oligo dT primer, MgCl₂, DTT, and HIV RT enzyme following the manufacturer's recommendations as closely as was allowable.

TABLE 7
cDNA synthesis reactions using HIV RT enzyme.

| Sample | RNA Used (Quantity) | dNTPs Added (Amount) |
|---|---|---|
| 1 | eGFP RNA (Unmodified) (1 μg) | — |
| 2 | eGFP RNA (Alpha-Thio-A) (1 μg) | — |
| 3 | eGFP RNA (Alpha-Thio-U) (1 μg) | — |
| 4 | eGFP RNA (5-Me—C and Pseudo-U) | — |
| | (1 μg) | |
| 5 | eGFP RNA (Unmodified) (1 μg) | 10 mM dNTPs (1 μL) |
| 6 | eGFP RNA (Alpha-Thio-A) (1 μg) | 10 mM dNTPs (1 μL) |
| 7 | eGFP RNA (Alpha-Thio-U) (1 μg) | 10 mM dNTPs (1 μL) |
| 8 | eGFP RNA (5-Me—C and Pseudo-U) | 10 mM dNTPs (1 μL) |
| | (1 μg) | |

All reactions received 2 μL 10× RT reaction buffer, 4 μL 25 mM MgCl₂, 2 μL 0.1 M DTT, 1 μL 50 μM Oligo dT, and 1 μL HIV RT enzyme (20U). The reactions were incubated at 37° C. for 50 minutes, as recommended by the enzyme supplier.

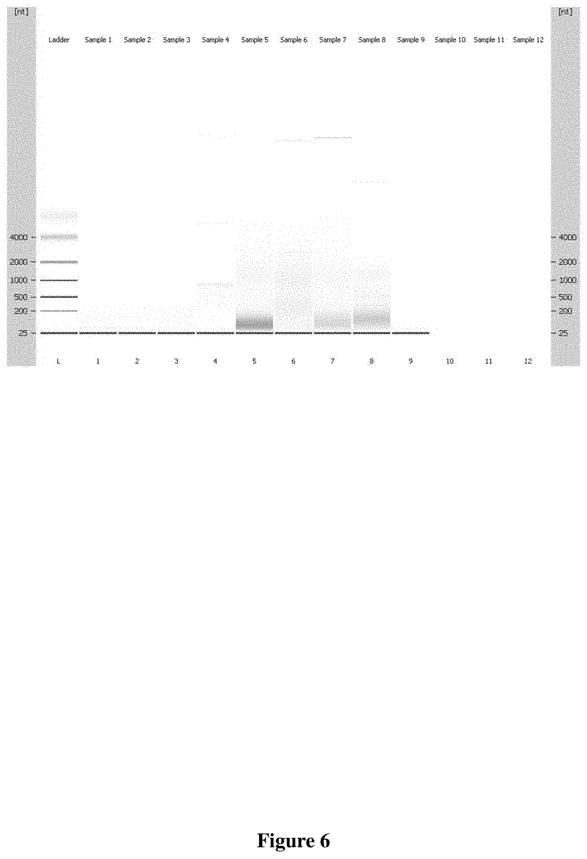

Results: The reactions from Table 7 were run on an Agilent Bioanalyzer 2100 RNA 6000 Nano Chip. The results are displayed in FIG. 6, Agilent Bioanalyzer results of cDNA synthesis assays. The results of the cDNA synthesis assays showed that the HIV RT enzyme did not generate cDNA from any of the templates tested. It appears that the RNase activity of the HIV RT enzyme (likely due to exogenous RNases or endogenous RNase H activity) is so powerful that the RNA is degraded before it can be converted to cDNA.

Worthington Biochemical was contacted about the possibility of exogenous RNase activity in the HIV RT product. The company agreed to send two different test lot aliquots of the product and made the recommendations to add more template to the reactions and decrease the amount of HIV RT enzyme used. These experimental changes were evaluated in the following assays.

TABLE 8
Two new HIV RT lot degradation assays on eGFP RNA (Unmodified) template

| | RNA Used | Reaction Buffer | Enzyme Used | Incubation |
|---|---|---|---|---|
| Sample | (Quantity) | Used (Quantity) | (Quantity) | Conditions |
| 1 | eGFP RNA | — | — | 37° C. 30 min, |
| | (Unmodified) (1 μg) | | | 70° C. 20 min |
| 2 | eGFP RNA | 10X NEB | — | 37° C. 30 min, |
| | (Unmodified) (15 μg) | Buffer 4 (1 μL) | | 70° C. 20 min |
| 3 | eGFP RNA | — | HIV RT 1 (10 U) | 37° C. 30 min, |
| | (Unmodified) (15 μg) | | | 70° C. 20 min |
| 4 | eGFP RNA | — | HIV RT 2 (10 U) | 37° C. 30 min, |
| | (Unmodified) (15 μg) | | | 70° C. 20 min |
| 5 | eGFP RNA | 10X NEB | HIV RT 1 (10 U) | 37° C. 30 min, |
| | (Unmodified) (15 μg) | Buffer 4 (1 μL) | | 70° C. 20 min |
| 6 | eGFP RNA | 10X NEB | HIV RT 2 (10 U) | 37° C. 30 min, |
| | (Unmodified) (15 μg) | Buffer 4 (1 μL) | | 70° C. 20 min |
| 7 | eGFP RNA | 10X NEB | — | 37° C. 30 min, |
| | (Unmodified) (1 μg) | Buffer 4 (1 μL) | | 70° C. 20 min |
| 8 | eGFP RNA | — | HIV RT 1 (10 U) | 37° C. 30 min, |
| | (Unmodified) (1 μg) | | | 70° C. 20 min |
| 9 | eGFP RNA | — | HIV RT 2 (10 U) | 37° C. 30 min, |
| | (Unmodified) (1 μg) | | | 70° C. 20 min |
| 10 | eGFP RNA | 10X NEB | HIV RT 1 (10 U) | 37° C. 30 min, |
| | (Unmodified) (1 μg) | Buffer 4 (1 μL) | | 70° C. 20 min |
| 11 | eGFP RNA | 10X NEB | HIV RT 2 (10 U) | 37° C. 30 min, |
| | (Unmodified) (1 μg) | Buffer 4 (1 μL) | | 70° C. 20 min |

HIV RT 1: Lot# X3N14535
HIV RT 2: Lot# X3E14292

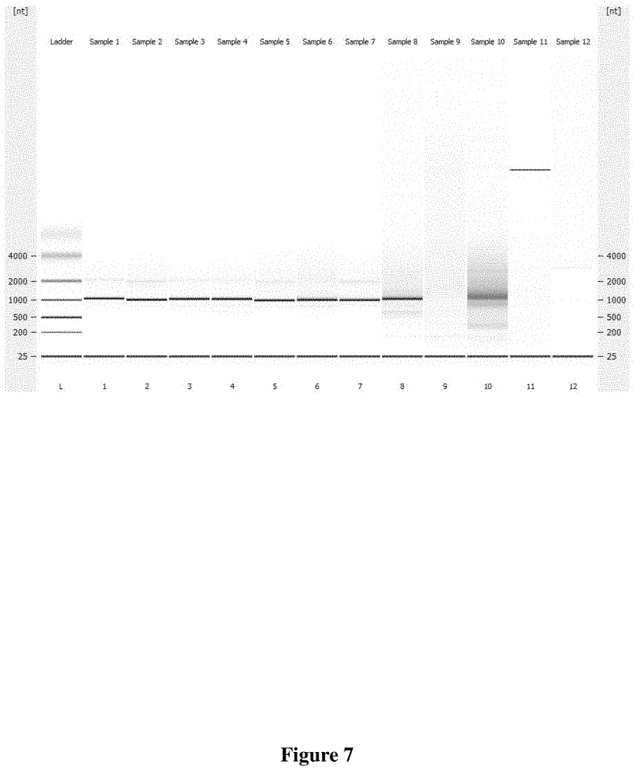

Results: The reactions from Table 8 were run on an Agilent Bioanalyzer 2100 RNA 6000 Nano Chip. The results are displayed below in FIG. 7, Agilent Bioanalyzer results of HIV RT degradation assays. It was apparent from these degradation assays that both of the two new lots of HIV RT enzyme had strong RNase activity and degraded the RNA template. HIV RT 1 showed less degradation than HIV RT 2. The lot to lot variation indicates that exogenous RNases are the likely source of the strong degradation. The strong degradation was minimized by the addition of 15 μg of template RNA. Another degradation assay was performed to evaluate the effects of increased template and decreased enzyme concentration on the original lot of HIV RT enzyme.

Additional degradation assays were undertaken to evaluate the effects of adding increased RNA template to reduce the previously seen degradation using the original lot of HIV RT enzyme.

TABLE 9
Original HIV RT lot degradation assays on eGFP RNA (Unmodified) template

| | RNA Used | Reaction Buffer | Enzyme Used | Incubation |
|---|---|---|---|---|
| Sample | (Quantity) | Used (Quantity) | (Quantity) | Conditions |
| 1 | eGFP RNA | — | — | 37° C. 30 min, |
| | (Unmodified) (1 μg) | | | 70° C. 20 min |
| 2 | eGFP RNA | 10X NEB | — | 37° C. 30 min, |
| | (Unmodified) (15 μg) | Buffer 4 (1 μL) | | 70° C. 20 min |
| 3 | eGFP RNA | — | Original HIV RT | 37° C. 30 min, |
| | (Unmodified) (15 μg) | | (10 U) | 70° C. 20 min |
| 4 | eGFP RNA | 10X NEB | Original HIV RT | 37° C. 30 min, |
| | (Unmodified) (15 μg) | Buffer 4 (1 μL) | (10 U) | 70° C. 20 min |
| 5 | eGFP RNA | 10X NEB | — | 37° C. 30 min, |
| | (Unmodified) (10 μg) | Buffer 4 (1 μL) | | 70° C. 20 min |
| 6 | eGFP RNA | — | Original HIV RT | 37° C. 30 min, |
| | (Unmodified) (10 μg) | | (10 U) | 70° C. 20 min |
| 7 | eGFP RNA | 10X NEB | Original HIV RT | 37° C. 30 min, |
| | (Unmodified) (10 μg) | Buffer 4 (1 μL) | (10 U) | 70° C. 20 min |

Original HIV RT: Lot# XR4A14736

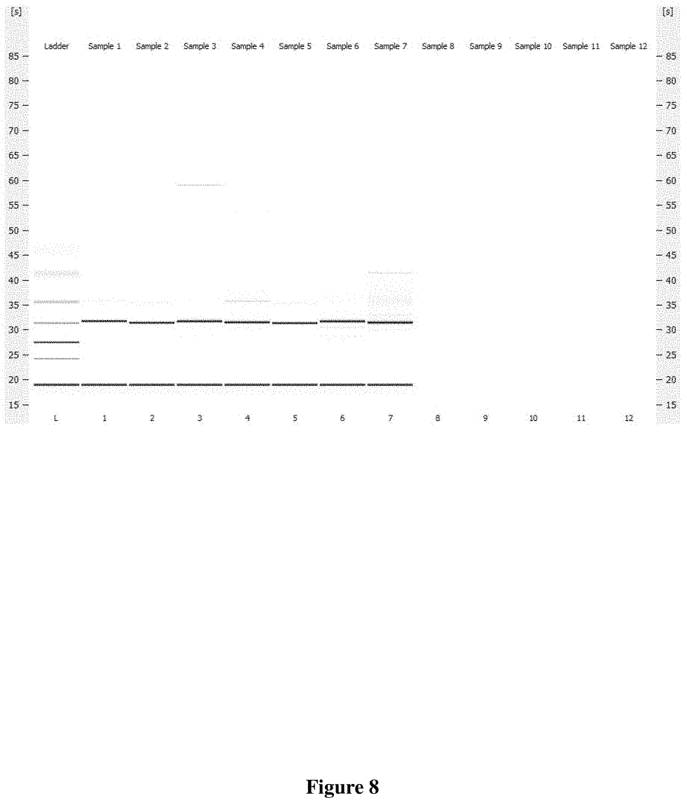

Results: The reactions from Table 9 were run on an Agilent Bioanalyzer 2100 RNA 6000 Nano Chip. The results are displayed in FIG. 8, Agilent Bioanalyzer results of HIV RT degradation assays. The addition of 10 μg and 15 μg of template RNA was enough to greatly minimize the previously observed degradation caused by the HIV RT enzyme. Given the ability to protect the RNA from degradation, the cDNA synthesis reactions were performed again.

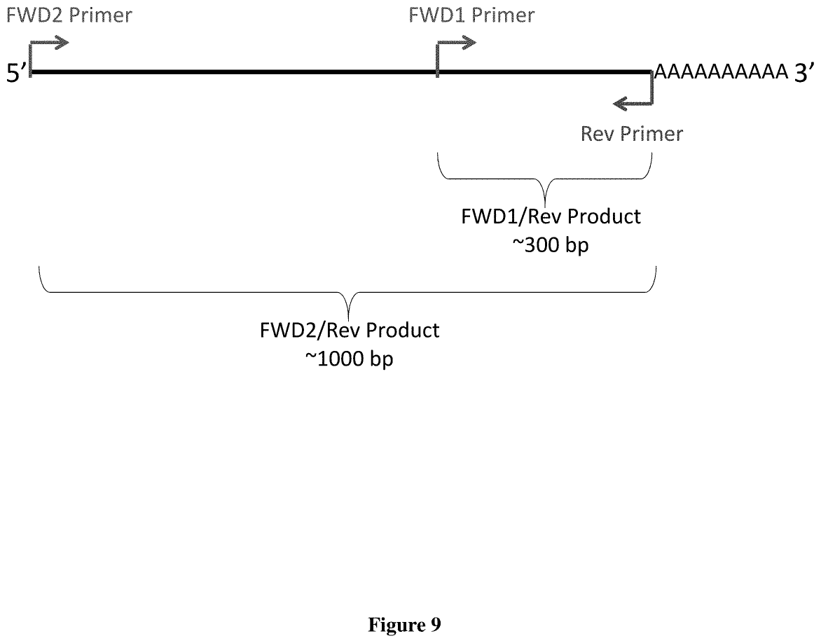

To further aid in functionality and specificity of the cDNA synthesis reactions, gene specific primers were designed. One set of primers (FWD2 and REV) were designed on the extreme ends of the template so as to create full length products. An internal primer (FW1) was also designed to work in conjunction with the REV primer to amplify from templates that may not have the 5′ end intact. Table 10 and FIG. 9 contain primer design information.

TABLE 10
Gene specific primer design information

| Primer Name | Primer Sequence |
|---|---|
| FWD1 | 5′-CTACCTGAGCACCCAGTCC-3′ |
| | (SEQ ID NO.: 1) |
| FWD2 | 5′-GGAAATAAGAGAGAAAAGAAGAG-3′ |
| | (SEQ ID NO.: 2) |
| REV | 5′-CTTCCTACTCAGGCTTTATTC-3′ |
| | (SEQ ID NO.: 3) |

FIG. 9 depicts a design schematic of eGFP RNA templates indicating placement of primers and expected amplicon sizes. Some of the RNAs were not modified by RT-PCR. RT has strong phage activity and was chopping up the poly A tail. Superscript does not have a strong phage activity. A poly A promoter sequence is present in the template in response to the phage activity of the RT.

Additional cDNA synthesis assays were undertaken to evaluate the use of increased RNA template input in an effort to reduce degradation, leading to increased cDNA yield. Additional positive control reactions were performed using the Superscript III enzyme (Invitrogen) and the eGFP RNA (Unmodified) in an attempt to establish that the RNA templates are able to be reverse transcribed. Reaction conditions are displayed in Table 11.

TABLE 11
cDNA synthesis reactions using increased RNA template

| | RNA Used | | Enzyme Used |
|---|---|---|---|
| Sample | (Quantity) | Primer Used | (Amount) |
| 1 | eGFP RNA (Unmodified) | 2 μM REV Primer (1 μL) | HIV RT (10 U) |
| | (10 μg) | | |
| 2 | eGFP RNA (Unmodified) | 50 μM Oligo dT (1 μL) | HIV RT (10 U) |
| | (10 μg) | | |
| 3 | eGFP RNA (Unmodified) | 2 μM REV Primer (1 μL) | HIV RT (10 U) |
| | (10 μg) | | |
| 4 | eGFP RNA (Unmodified) | 2 μM REV Primer (1 μL) | Superscript III (200 U) |
| | (10 μg) | | |
| 5 | eGFP RNA (Unmodified) | 50 μM Oligo dT (1 μL) | Superscript III (200 U) |
| | (10 μg) | | |

All reactions received 2 μL 10× RT reaction buffer, 4 μL 25 mM MgCl₂, 2 μL 0.1 M DTT, and 1 μL 10 mM dNTPs. In an effort to eliminate exogenous RNase activity that could be in the HIV RT enzyme, RNase OUT enzyme was added to one reaction. RNase OUT is known to inhibit a wide variety of RNase activity and is recommended by Invitrogen for addition cDNA synthesis reactions. All reactions had an initial primer annealing step of 65° C. for 5 minutes. The reactions containing HIV RT were incubated at 37° C. for 1 hour and the reactions containing Superscript III enzyme were incubated at 50° C. for 1 hour. All reactions were incubated at 85° C. for 5 minutes to terminate cDNA synthesis.

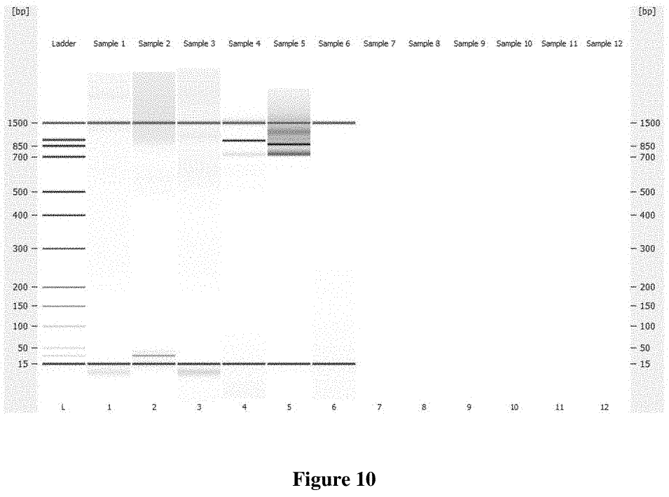

Results: The reactions from Table 11 were run on an Agilent Bioanalyzer 2100 DNA 1000 Chip. The results are displayed in FIG. 10, Agilent Bioanalyzer results of cDNA synthesis reactions. The reactions containing the Superscript III enzyme produced cDNA product using either the Oligo dT or gene specific priming mechanism. None of the HIV RT reactions produced visible cDNA product. The reaction containing the RNase OUT enzyme produced no cDNA product, indicating that there was either no exogenous RNases present in the HIV RT (the observed RNA degradation was a result of the inherent RNase H activity) or there was sufficient quantity of exogenous RNases to overcome the inhibition of the RNase OUT enzyme.

### End Point PCR

To determine whether cDNA was being produced by the HIV RT enzyme but at levels below the detection threshold of the Agilent Bioanalyzer, end point PCR assays were conducted using the gene specific primer sets displayed in Table 10 and FIG. 9. HIV RT and Superscript III cDNA reactions using the gene specific primer were used as templates for the PCR reactions. To assess the RNA stocks received from TriLink Biotechnologies for potential residual DNA contamination, control reactions were performed using 100 ng of the stock RNA.

TABLE 12
End point PCRs of cDNA reactions

| Sample | Template cDNA reaction | Primer Set Used |
|---|---|---|
| 1 | HIV RT | FWD1 + REV |
| 2 | HIV RT with RNase OUT | FWD1 + REV |
| 3 | Superscript III | FWD1 + REV |
| 4 | eGFP RNA (Unmodified) (100 ng) | FWD1 + REV |
| 5 | HIV RT | FWD2 + REV |
| 6 | HIV RT with RNase OUT | FWD2 + REV |
| 7 | Superscript III | FWD2 + REV |
| 8 | eGFP RNA (Unmodified) (100 ng) | FWD2 + REV |

Each reaction received 25 μL GoTaq green master mix (Promega), 2.5 μL of each 10 μM forward and reverse primer, and 1 μL template. The reactions were brought to 50 μL total volume with nuclease-free water. The reactions were placed on a thermal cycler and amplified using the following parameters:

| | | | | | |
|---|---|---|---|---|---|
| 1. | 95° C. | 1 | cycle | 5 | min |
| 2. | 95° C. | 35 | cycles | 30 | sec |
| 3. | 48° C. | 35 | cycles | 30 | sec |

| | | | | |
|---|---|---|---|---|
| 4. | 72° C. | 35 | cycles | 1 min 15 sec |
| 5. | 4° C. | 1 | cycle | Hold |

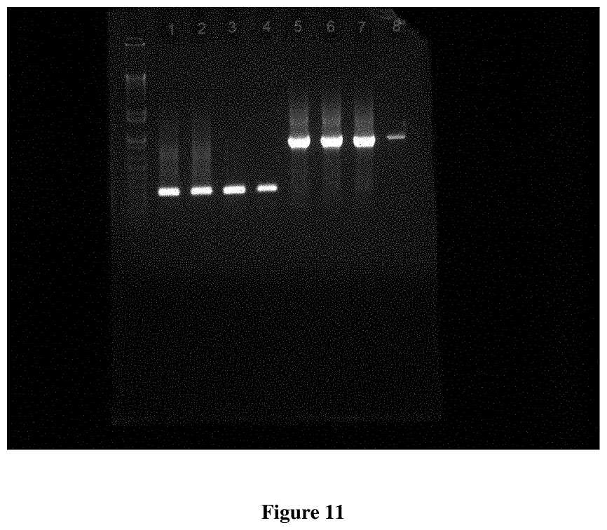

Following amplification, the PCR reactions were analyzed and resolved by 1% agarose gel electrophoresis. FIG. 11, agarose gel electrophoresis of end point PCR reactions. The PCR reactions all showed amplification of the expected sizes (˜300 bp for FWD1 and ˜1000 bp for FWD2) indicating that there was near full length DNA in the PCR reaction tubes. The RNA stock template control reactions also generated amplicon, indicating the presence of residual DNA in the stocks. It was not known whether the amplicons generated were a result of amplification from cDNA templates or simply from the residual DNA. To evaluate this, the RNA stock templates received from TriLink Biotechnologies needed to be DNase treated to fully remove any residual DNA entities.

### Example 4

### Removal of Contaminating DNA from 4 RNA Templates

To fully remove the DNA contamination in the RNA stocks, the following general protocol was used. 10 μg of each RNA was incubated with DNase I reaction buffer (NEB) and 2U of DNase I enzyme (NEB) in 100 μL total volume at 37° C. for 15 minutes followed by 75° C. for 10 minutes to inactivate the enzyme.

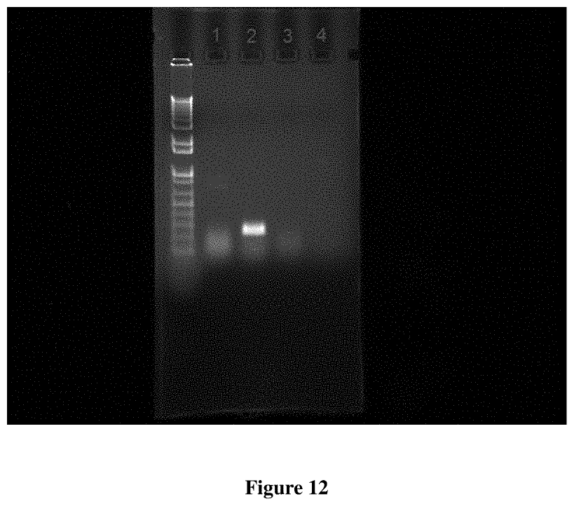

The eGFP RNA (Unmodified) template was tested first to determine how many iterations of DNase I treatment would be necessary to fully remove the DNA contamination. Each iteration included both positive and negative controls. The positive controls used 200 ng of stock RNA template and the negative control reactions had no template added. The results of the four iterations performed are displayed in FIGS. 12-15. The order of samples on each gel was consistent and is displayed in Tables 13-16.

TABLE 13
First Iteration of eGFP RNA (Unmodified) DNase treatment

| Sample | | |
|---|---|---|
| Number | Template Used (amount) | Primers Used |
| 1 | eGFP RNA (Unmodified) Stock 200 ng | FWD2 + REV |
| 2 | eGFP RNA (Unmodified) from first iteration | FWD1 + REV |
| 3 | eGFP RNA (Unmodified) from first iteration | FWD2 + REV |
| 4 | — | FWD1 + REV |

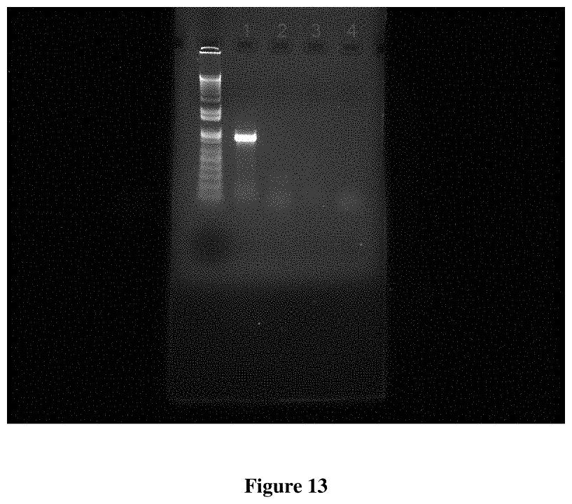

FIG. 12 depicts the agarose gel electrophoresis results from Table 13. There was no full length product generated from the FWD2 reaction. However, there was amplicon generated by the FWD1 reaction, indicating that there was still a non-full length contaminating DNA present. The sample required another iteration of DNase treatment. Data for the second iteration is displayed in Table 14 and FIG. 13.

TABLE 14
Second Iteration of eGFP RNA (Unmodified) DNase treatment

| Sample | | |
|---|---|---|
| Number | Template Used (amount) | Primers Used |
| 1 | eGFP RNA (Unmodified) Stock 200 ng | FWD2 + REV |
| 2 | eGFP RNA (Unmodified) Stock from second | FWD1 + REV |
| | iteration | |
| 3 | eGFP RNA (Unmodified) Stock from second | FWD2 + REV |
| | iteration | |
| 4 | — | FWD1 + REV |

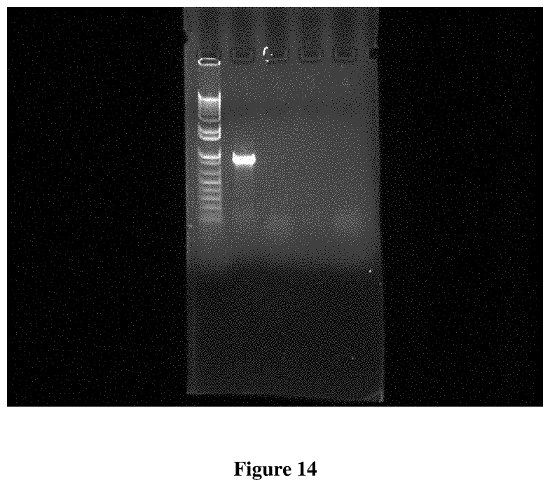

FIG. 13 depicts the agarose gel electrophoresis results from Table 14. There was no full length product generated from the FWD2 reaction. However, there was amplicon generated by the FWD1 reaction, indicating that there was still a non-full length contaminating DNA present. There was a visible decrease in the amount of amplicon produced from the first to second DNase iteration indicating that contaminating DNA is being removed. The sample required another iteration of DNase treatment. Data for the third iteration is displayed in Table 15 and FIG. 14.

TABLE 15
Third Iteration of eGFP RNA (Unmodified) DNase treatment

| Sample | | |
|---|---|---|
| Number | Template Used (amount) | Primers Used |
| 1 | eGFP RNA (Unmodified) Stock 200 ng | FWD2 + REV |
| 2 | eGFP RNA (Unmodified) Stock from third | FWD1 + REV |
| | iteration | |
| 3 | eGFP RNA (Unmodified) Stock from third | FWD2 + REV |
| | iteration | |
| 4 | — | FWD1 + REV |

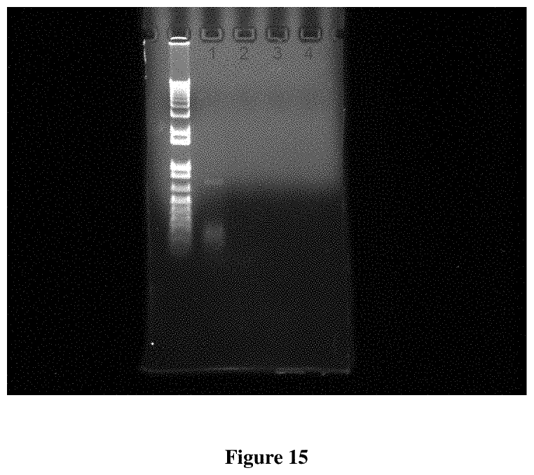

FIG. 14 depicts the agarose gel electrophoresis results from Table 15. There was no full length product generated from the FWD2 reaction. However, there was amplicon generated by the FWD1 reaction, indicating that there was still a non-full length contaminating DNA present. There was a visible decrease in the amount of amplicon produced from the second to third DNase iteration indicating that contaminating DNA is being further removed. The sample required another iteration of DNase treatment. Data for the fourth iteration is displayed in Table 16 and FIG. 15.

TABLE 16
Fourth Iteration of eGFP RNA (Unmodified) DNase treatment

| Sample | | |
|---|---|---|
| Number | Template Used (amount) | Primers Used |
| 1 | eGFP RNA (Unmodified) Stock 200 ng | FWD2 + REV |
| 2 | eGFP RNA (Unmodified) Stock from fourth | FWD1 + REV |
| | iteration | |
| 3 | eGFP RNA (Unmodified) Stock from fourth | FWD2 + REV |
| | iteration | |
| 4 | — | FWD1 + REV |

FIG. 15 depicts the agarose gel electrophoresis results from Table 16. There was no full length product generated from either the FWD1 or FWD2 reactions, indicating that there was no contaminating DNA remaining in the sample. This result established that four iterations of DNase treatment were required to fully remove contaminating DNA from the RNA stock.

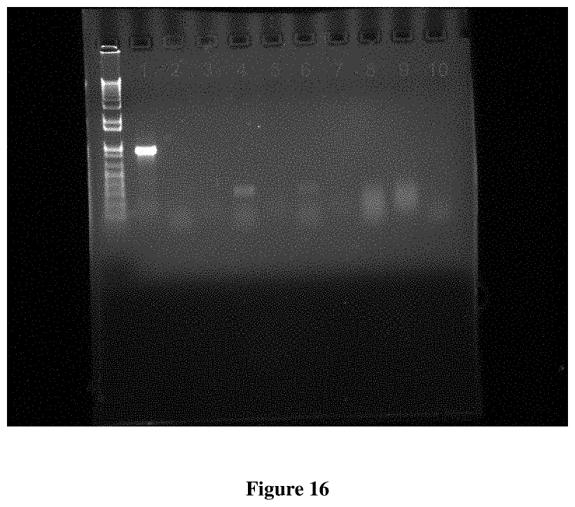

All four RNA templates were subjected to three rounds of DNase treatment before being subjected to end point PCR under the conditions described above followed by analysis by agarose gel electrophoresis. 15 μg of starting RNA stock template was added to the first iteration of DNase treatment. The results are displayed in Table 17 and FIG. 16.

TABLE 17
Third Iteration of all four RNA templates DNase treatment

| Sample | | |
|---|---|---|
| Number | Template Used (amount) | Primers Used |
| 1 | eGFP RNA (Unmodified) Stock 200 ng | FWD2 + REV |
| 2 | eGFP RNA (Unmodified) Stock from third iteration | FWD1 + REV |
| 3 | eGFP RNA (Unmodified) Stock from third iteration | FWD2 + REV |
| 4 | eGFP RNA (Alpha-Thio-A) Stock from third iteration | FWD1 + REV |
| 5 | eGFP RNA (Alpha-Thio-A) Stock from third iteration | FWD2 + REV |
| 6 | eGFP RNA (Alpha-Thio-U) Stock from third iteration | FWD1 + REV |
| 7 | eGFP RNA (Alpha-Thio-U) Stock from third iteration | FWD2 + REV |
| 8 | eGFP RNA (5-Me—C and Pseudo-U) Stock from third iteration | FWD1 + REV |
| 9 | eGFP RNA (5-Me—C and Pseudo-U) Stock from third iteration | FWD2 + REV |
| 10 | — | FWD1 + REV |

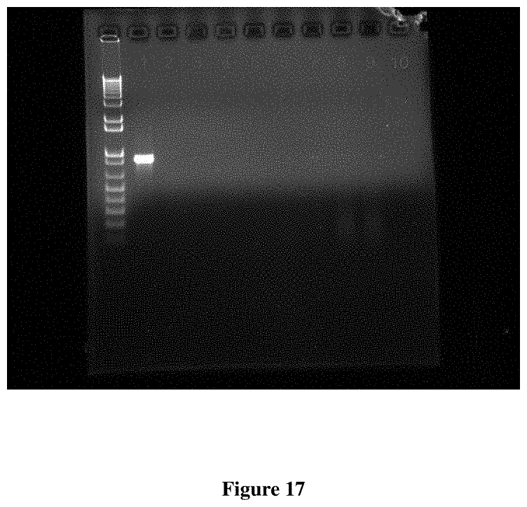

FIG. 16 depicts the gel electrophoresis results from Table 17. There was still contaminating DNA present in the eGFP RNA (Alpha-Thio-A), eGFP RNA (Alpha-Thio-U), and eGFP RNA (5-Me-C and Pseudo-U) samples. These three samples were subjected to a fourth iteration of DNase treatment. A new aliquot of eGFP RNA (5-Me-C and Pseudo-U) were subjected to a first iteration of DNase treatment as well. The results are displayed in Table 18 and FIG. 17.

TABLE 18
Fourth Iteration of DNase treatment

| Sample | | |
|---|---|---|
| Number | Template Used (amount) | Primers Used |
| 1 | eGFP RNA (Unmodified) Stock 200 ng | FWD2 + REV |
| 2 | eGFP RNA (Alpha-Thio-A) Stock from fourth iteration | FWD1 + REV |
| 3 | eGFP RNA (Alpha-Thio-A) Stock from fourth iteration | FWD2 + REV |
| 4 | eGFP RNA (Alpha-Thio-U) Stock from fourth iteration | FWD1 + REV |
| 5 | eGFP RNA (Alpha-Thio-U) Stock from fourth iteration | FWD2 + REV |
| 6 | eGFP RNA (5-Me—C and Pseudo-U) Stock from fourth iteration | FWD1 + REV |
| 7 | eGFP RNA (5-Me—C and Pseudo-U) Stock from fourth iteration | FWD2 + REV |
| 8 | eGFP RNA (5-Me—C and Pseudo-U) Stock from first iteration | FWD1 + REV |
| 9 | eGFP RNA (5-Me—C and Pseudo-U) Stock from first iteration | FWD2 + REV |
| 10 | — | FWD1 + REV |

FIG. 17 depicts the agarose gel electrophoresis results from Table 18. The contaminating DNA was removed from all of the RNA templates enabling the RNA templates to be used as templates for cDNA synthesis reactions.

### Example 5

### cDNA Synthesis

The four DNA-free RNA templates were used as templates in cDNA synthesis reactions. Each RNA template was incubated with reaction buffer, dNTPs, gene specific primer, MgCl₂, DTT, and either HIV RT or Superscript III enzyme following the manufacturer's recommendations as closely as was allowable.

TABLE 19
cDNA synthesis reactions of DNase free RNA templates

| | | | Enzyme Used |
|---|---|---|---|
| Sample | RNA Used | Primer Used | (Amount) |
| 1 | HeLa RNA | 50 μM Oligo dT | Superscript III |
| | (10 ng) | (1 μL) | (200 U) |
| 2 | eGFP RNA | 2 μM REV Primer | Superscript III |
| | (Unmodified) | (1 μL) | (200 U) |
| 3 | eGFP RNA | 2 μM REV Primer | HIV RT (10 U) |
| | (Unmodified) | (1 μL) | |
| 4 | eGFP RNA | 2 μM REV Primer | HIV RT (10 U) |
| | (Alpha-Thio-A) | (1 μL) | |
| 5 | eGFP RNA | 2 μM REV Primer | HIV RT (10 U) |
| | (Alpha-Thio-U) | (1 μL) | |
| 6 | eGFP RNA | 2 μM REV Primer | HIV RT (10 U) |
| | (5-Me—C and | (1 μL) | |
| | Pseudo-U) | | |

All reactions received 2 μL 10× RT reaction buffer, 4 μL 25 mM MgCl₂, 2 μL 0.1 M DTT, and 1 μL 10 mM dNTPs. A positive control reaction in the kit was performed using HeLa cell RNA and Oligo dT. All reactions had an initial primer annealing step of 65° C. for 5 minutes. The reactions containing HIV RT enzyme were incubated at 37° C. for 1 hour and the reactions containing Superscript III enzyme were incubated at 50° C. for 1 hour. All reactions were incubated at 85° C. for 5 minutes to terminate the cDNA synthesis.

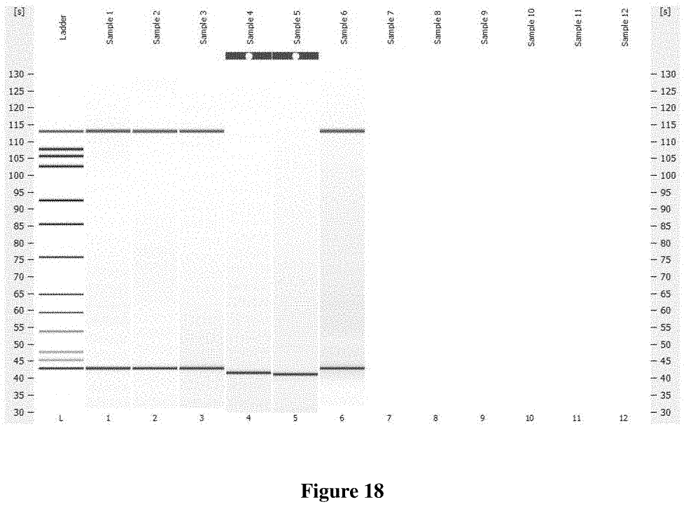

Results: The reactions from Table 19 above were run on an Agilent Bioanalyzer 2100 DNA 1000 Chip. The results are displayed in FIG. 18, Agilent Bioanalyzer results of cDNA synthesis assays. There was no visible cDNA produced by any of the reactions. The absence of cDNA product from the positive control HeLa reaction can be explained by the addition of only 10 ng of starting template. This is below the detectable limit of the Agilent Bioanalyzer. A further end point PCR using gene specific primers could be developed and performed to evaluate whether this reaction did produce cDNA.

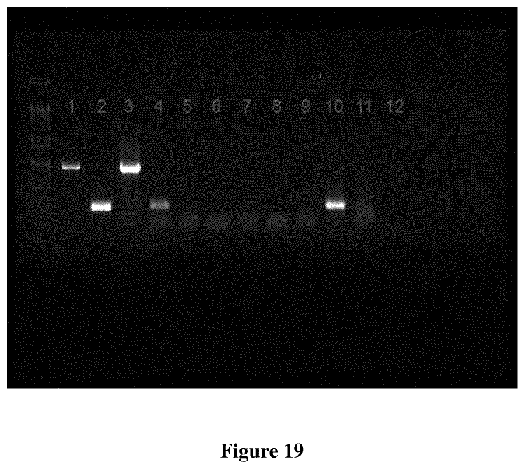

End point PCR assays were conducted on the other cDNA reactions to determine if any cDNA was generated by the reactions. The PCR conditions were the same as described above and analyzed by agarose gel electrophoresis. 1 μL of each cDNA reaction was used as template for PCR. Results from this assay are displayed below in Table 20 and FIG. 19.

TABLE 20
End Point PCR assays on cDNA reactions

| Sample | | |
|---|---|---|
| Number | Template Used (amount) | Primers Used |
| 1 | eGFP RNA (Unmodified) Stock 200 ng | FWD2 + REV |
| 2 | DNA free eGFP RNA (Unmodified) with | FWD1 + REV |
| | Superscript III | |
| 3 | DNA free eGFP RNA (Unmodified) with | FWD2 + REV |
| | Superscript III | |
| 4 | DNA free eGFP RNA (Unmodified) with HIV | FWD1 + REV |
| | RT | |
| 5 | DNA free eGFP RNA (Unmodified) with HIV | FWD2 + REV |
| | RT | |
| 6 | DNA Free eGFP RNA (Alpha-Thio-A) with | FWD1 + REV |
| | HIV RT | |
| 7 | DNA Free eGFP RNA (Alpha-Thio-A) with | FWD2 + REV |
| | HIV RT | |
| 8 | DNA Free eGFP RNA (Alpha-Thio-U) with | FWD1 + REV |
| | HIV RT | |
| 9 | DNA Free eGFP RNA (Alpha-Thio-U) with | FWD2 + REV |
| | HIV RT | |
| 10 | DNA Free eGFP RNA (5-Me—C and | FWD1 + REV |
| | Pseudo-U) with HIV RT | |
| 11 | DNA Free eGFP RNA (5-Me—C and | FWD2 + REV |
| | Pseudo-U) with HIV RT | |
| 12 | — | FWD1 + REV |

FIG. 19 depicts the agarose gel electrophoresis results from Table 20. The cDNA reaction containing DNA-free eGFP RNA (Unmodified) and Superscript III enzyme created amplicons from both FWD1 (Sample 2) and FWD2 (Sample 3) primer sets, indicating that full length cDNA was generated. There was amplification seen in the FWD1 reactions from DNA free eGFP RNA (Unmodified) (sample 4) and DNA Free eGFP RNA (5-Me-C and Pseudo-U) (sample 10), but not in their FWD2 counterpart reactions, indicating that there was cDNA generated by the reaction, but not full length. No amplification was observed in the reactions containing DNA free eGFP RNA (Alpha-Thio-A) (samples 6-7) or DNA free eGFP RNA (Alpha-Thio-U) (samples 8-9) indicating that the RNA in those cDNA reactions was degraded to below 300 bp in length by the HIV RT enzyme.

The data suggests that the HIV RT enzyme is capable of producing truncated cDNA from some of the RNA templates but the RNase activity in the enzyme is too strong to allow for full length cDNA generation.

### Example 6

### Gene Design

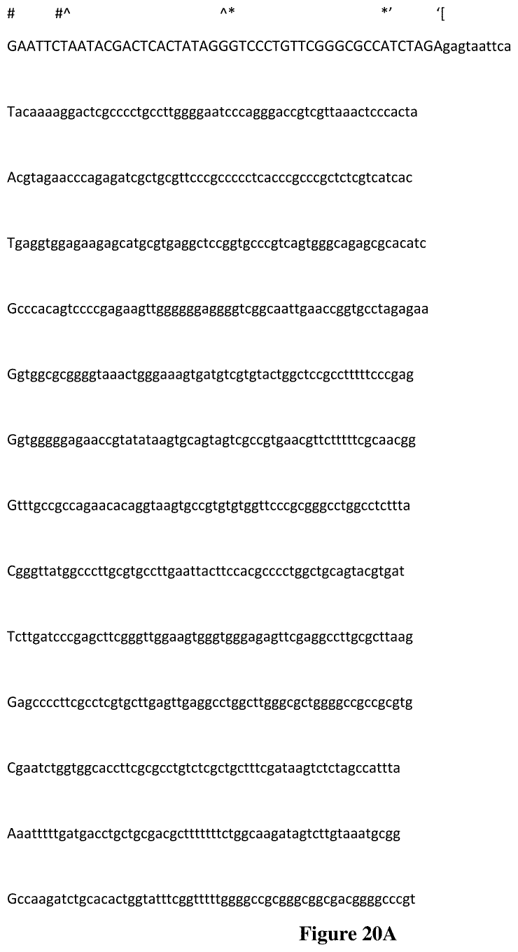

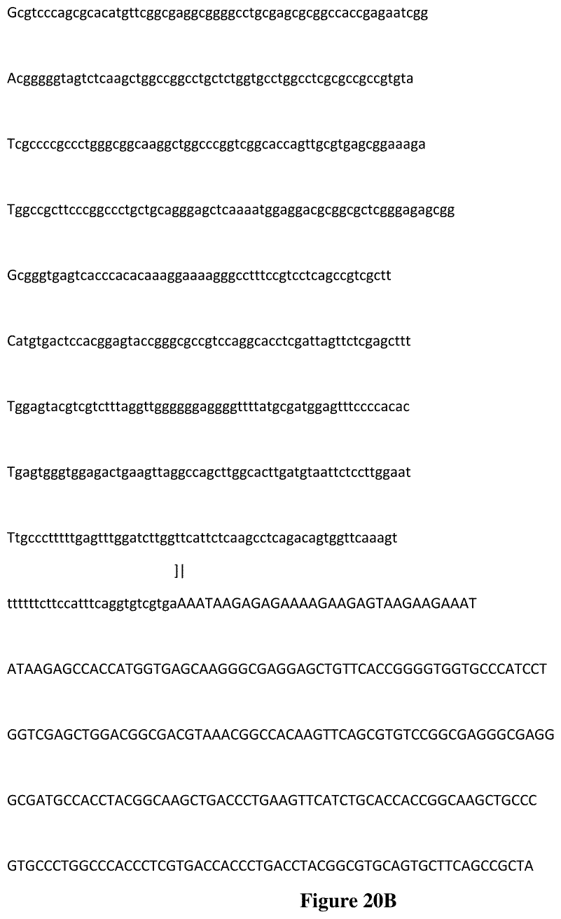

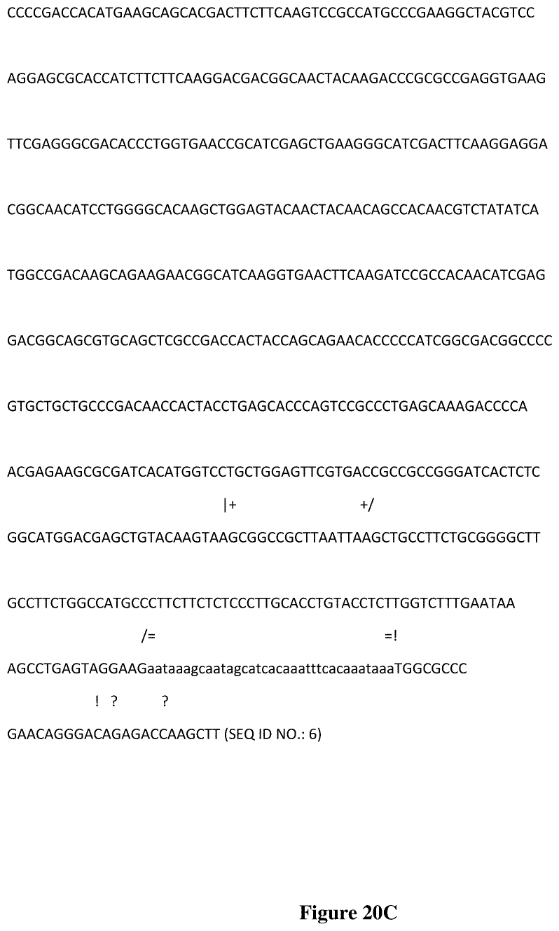

The gene was designed using the following specifications: a) a plasmid which is transcribed under the control of the T7 promoter yielding an RNA (uncapped and not polyadenylated) which after undergoing RT PCR will yield a double stranded gene. Transcription of that double stranded DNA (gene) will yield capped and polyadenylated mRNA transcripts with the indicated 5′ and 3 UTR sequence. b) a primer binding site on the resulting RNA transcribed from the plasmid such that the forward and reverse primer that bind to the RNA in RT PCR have the same sequence. The PBS as listed here will be extrapolated to the plasmid. The primer binding sequence on the RNA template strand is 5′ UGG CGC CCG AAC AGG GAC 3′ (SEQ ID NO.: 4)and the primer binding sequence that binds the RNA template strand is 3′ ACCGCGGGCTTGTCCCTG 5′ (SEQ ID NO.: 5). C) The resulting RNA after undergoing RT PCR will yield a double stranded DNA gene under the EF1α promoter. The EF1α promoter can be replaced by another promoter of choice using the restriction site. d) the resulting RNA after undergoing RT PCR will yield the EGFP gene under the control of the EF1α promoter. e) the EGFP gene can be replaced by another gene (ORF) of choice as a result of the restriction site. f) the EGFP gene when transcribed will have the 5′ UTR and 3′ UTR sequence for stabilization of the resulting mRNA. g) The RNA transcribed from this plasmid can be wild type or modified with various base modifications. H) component order within the plasmid is 1) t7 promoter 2) primer binding site 3) unique restriction enzyme site 4) EF1α promoter 5) 5′ UTR 6) unique restriction enzyme site 7) EGFP 8) unique restriction enzyme site 9) 3′ UTR 10) poly A signal 11) reverse complement of primer binding site. FIG. 20 depicts the sequence for gene synthesis. The various sites and sequences are indicated using the following: GAATTC EcoRI restriction site (#); T7 promoter (\hat{ }); forward primer sequence (*); XbaI site (′); Ef1α promoter ([and]); 5′ UTR ({and}); unique NcoI site CCATGG (contains start codon) (|); EGFP (\); Unique NotI and PacI sites (+); 3′ UTR (/); SV40 poly A signal (=); Reverse primer binding site (!); and BsaI runoff linearization enzyme (?).

The sequence was linearized with BsaI. BsaI cuts twice in the vector. FIGS. 20A-20C (SEQ ID NO.: 6).

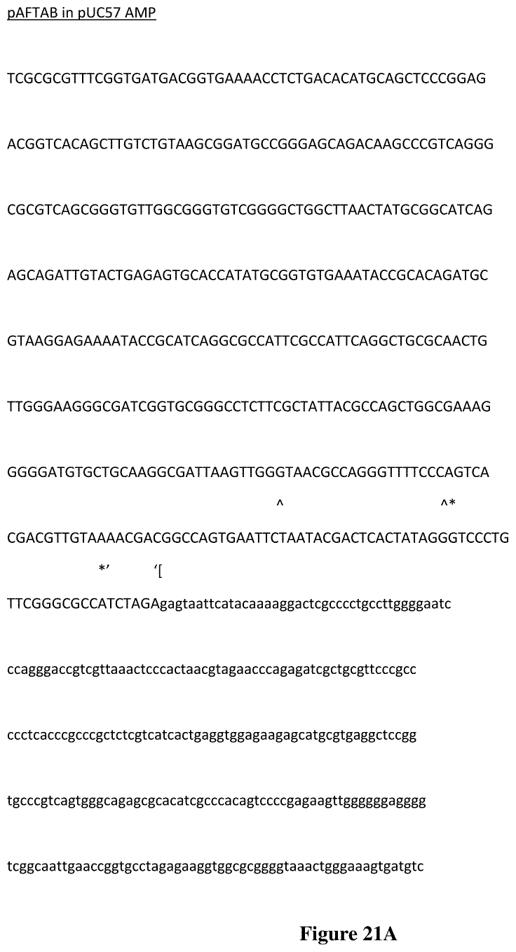

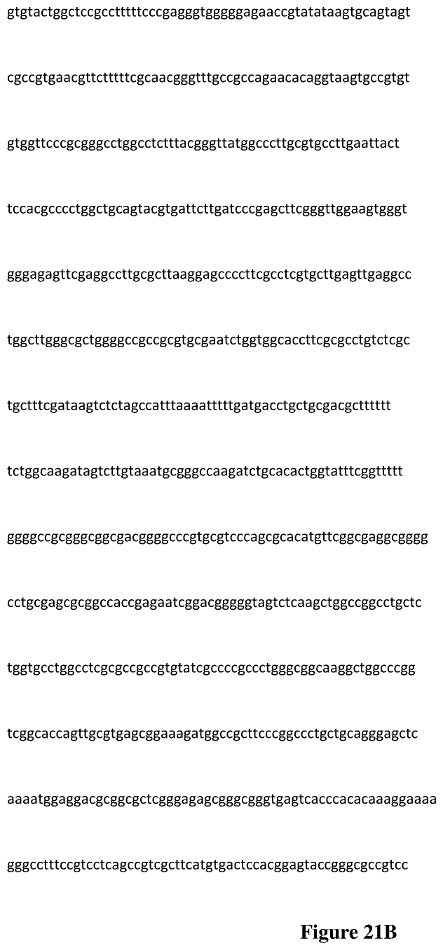

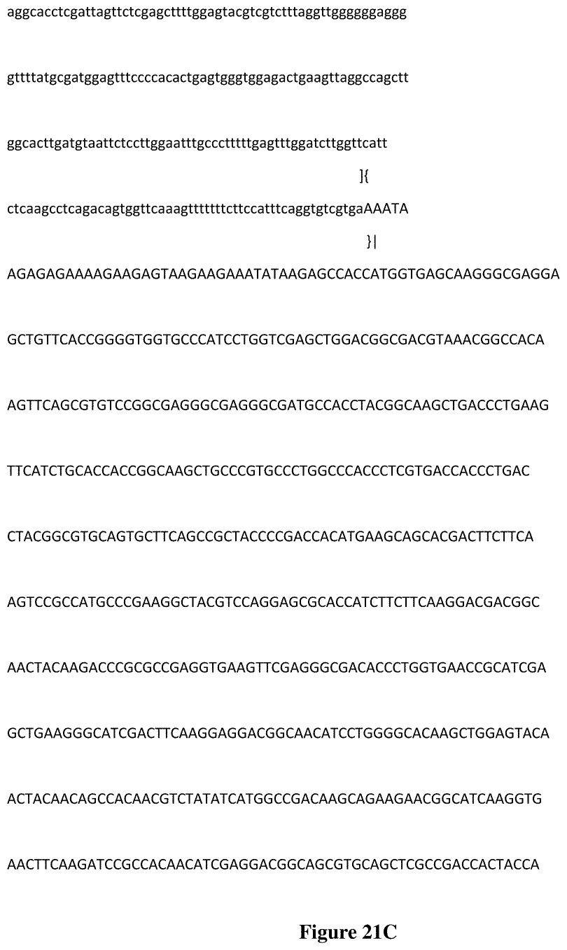

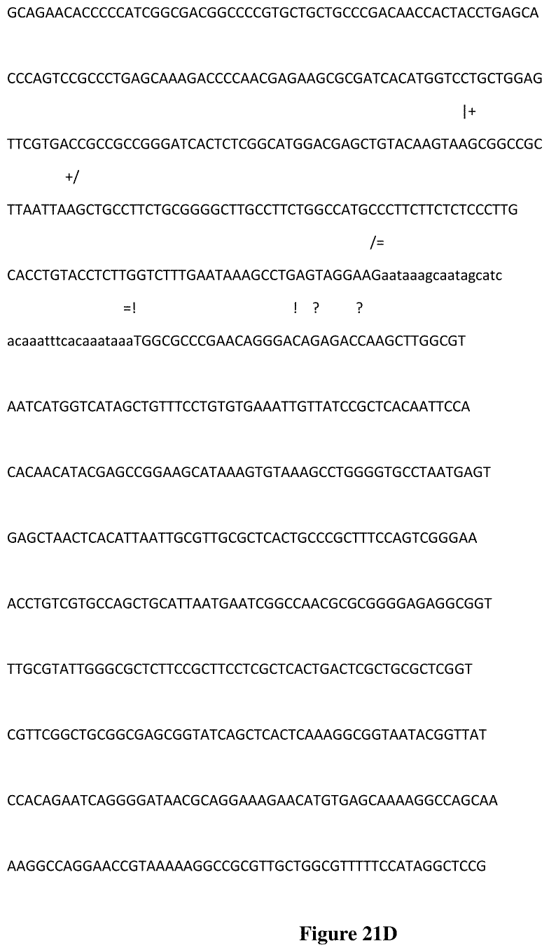

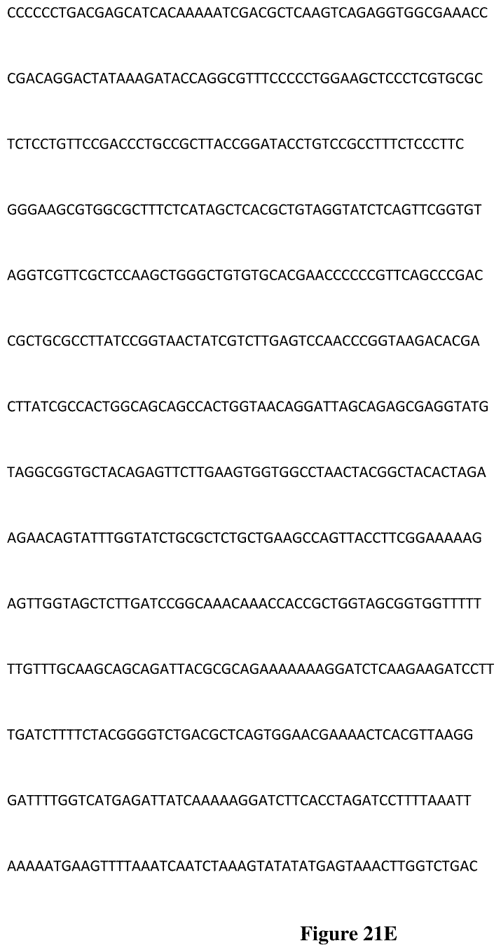

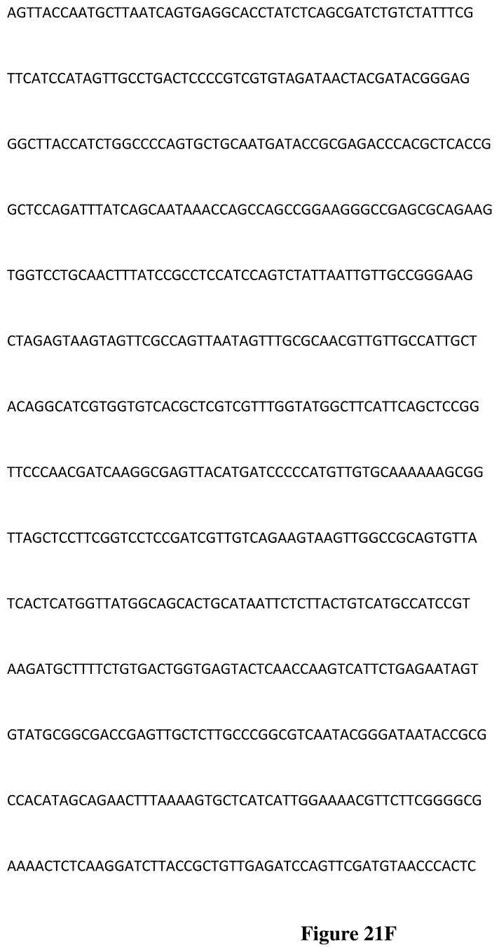

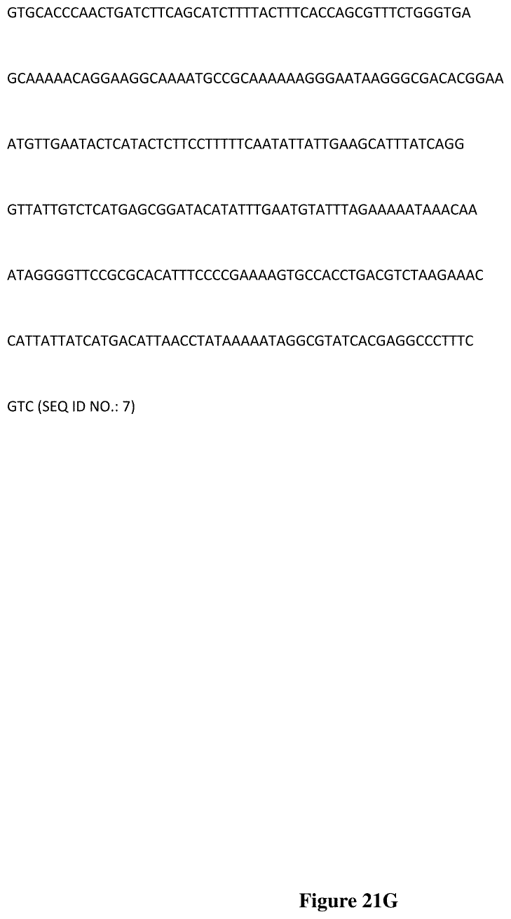

The sequence was cut with EcoRI and HindIII and ligate into EcoRI/HindIII digested pUC57 AMP, resulting in pAFTAB in pUC57 AMP plasmid. FIGS. 21A-21G (SEQ ID NO.: 7).

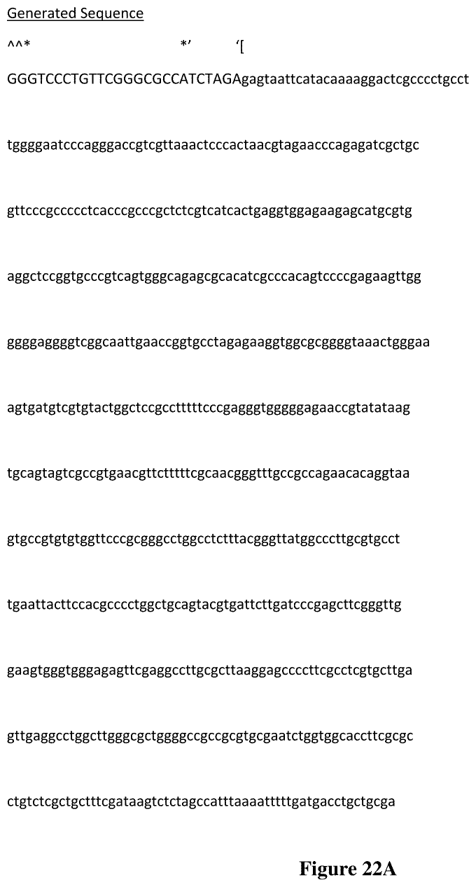

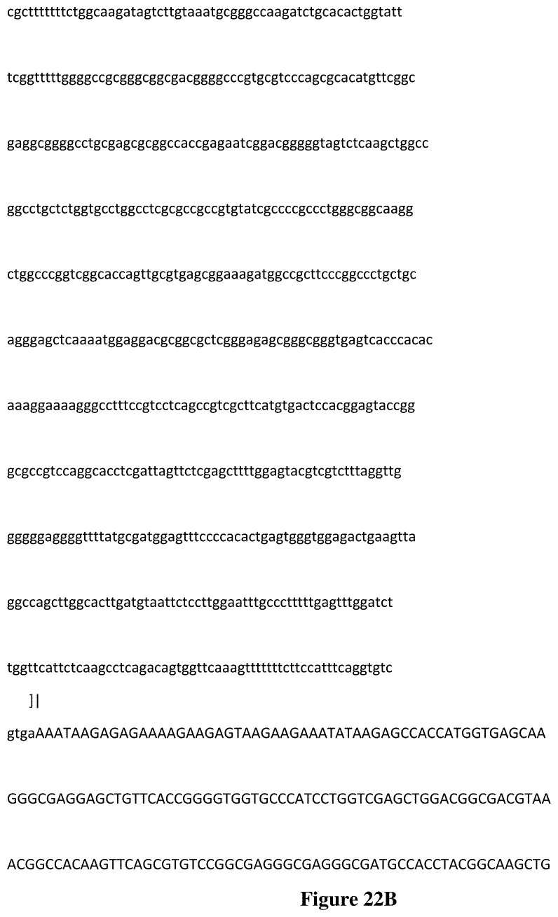

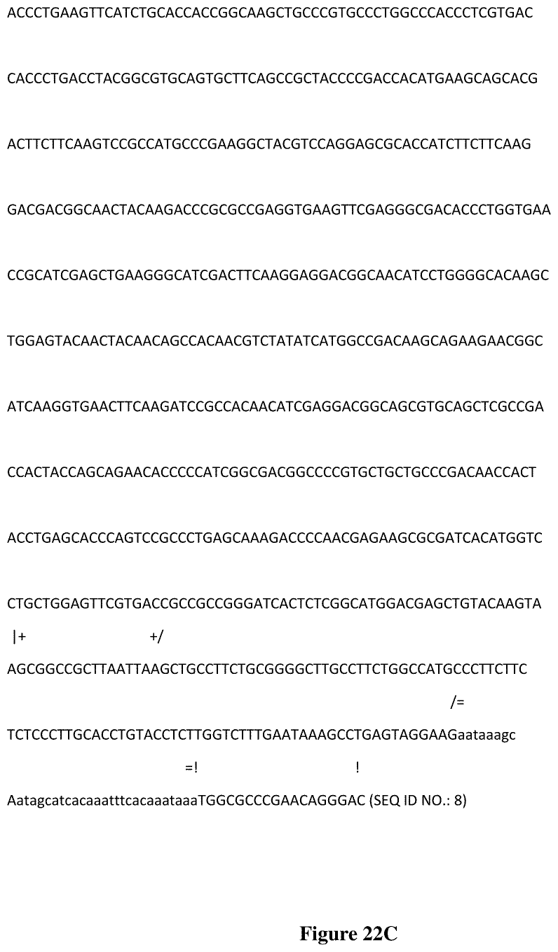

The sequence that will be generated is depicted in FIGS. 22A-22C (SEQ ID NO.: 8).

A diagram of the pAFTAB plasmid is depicted in FIG. 23.

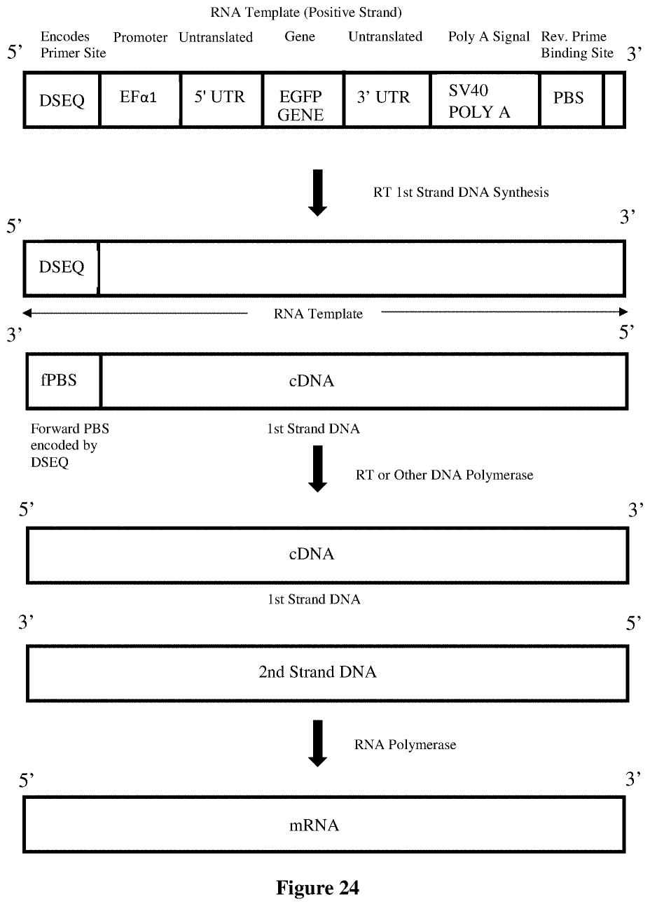

FIG. 24 is a chart displaying obtaining a desired mRNA from the RNA template. Reverse transcriptase generates complementary DNA from the RNA template. A second strand of DNA is generated using RT, DNA polymerase, or other enzyme. Double-stranded DNA encoding the gene, EGFP, under the EFα1 promoter is generated. RNA polymerase is used to generate products of the double-stranded DNA. Products of this DNA will be EGFP mRNA with a polyadenylation signal and a 5′ and 3′ UTR.

### Example 7

### Sequencing of RNA Samples

### DNase Treatment and RNA Purification:

Samples were DNase treated using the Qiagen RNase-Free DNase Set (Qiagen Catalog #79254) followed by purification using the Qiagen RNeasy Mini Kit (Qiagen Catalog #74104) and manufacturer's instructions. To eliminate potential gDNA contamination, each RNA sample was DNase treated four times.

cDNA Library Generation:

cDNA was generated using Invitrogen's SuperScript III First-Strand Synthesis System for RT-PCR (Invitrogen Catalog #18080-051) or a recombinant HIV Reverse Transcriptase (HIV RT, Worthington Biochemical Corporation Catalog #LS05003). All reactions received 2 μl 10 RT reaction buffer, 4 μl 25 mM MgCl2, 2 μl 0.1 M DTT, 1 μl 10 mM dNTPs, and 2 μM primer (8 μM when Phosphorothioate Primer used). For reactions using Superscript III, 200 U enzyme was used. Reactions using HIV RT contained 10 U enzyme. Different concentrations of RNA template were used, ranging from 1.5 to 5 μg/reaction. Table 21. No-RT and no-template controls were set up during reverse transcription. Reactions using Superscript III enzyme were incubated at 65° C. for 5 minutes, 50° C. for 60 minutes, and 85° C. for 5 minutes. Reactions using HIV RT were incubated at 65° C. for 5 minutes, 37° C. for 60 minutes, and 85° C. for 5 minutes. HIV RT was tested in the presence and absence of beta-thujaplicinol.

TABLE 21
Samples

| Sample Type | Sample ID |
|---|---|
| eGFP Transcript | Wild Type |
| eGFP Transcript | Alpha-Thio-Uridine |
| eGFP Transcript | 2-Thio-U |
| eGFP Transcript | 5MeC, PseudoU |

TABLE 22
PCR/Sequencing Primers
Primers for RT

| | |
|---|---|
| PBS Primer DNA | GTCCCTGTTCGGGCGCCA (SEQ ID NO.: 9) |
| PBS Primer RNA | GUCCCUGUUCGGGCGCCA (SEQ ID NO.: 10) |
| SV40 Primer DNA | TTTATTTGTGAAATTTGTGATGCTATTGCTTTATT (SEQ |
| | ID NO.: 11) |
| SV40 Primer RNA | UUUAUUUGUGAAAUUUGUGAUGCUAUUGCUUUAUU |
| | (SEQ ID NO.: 12) |
| Phosphorothioate | TTTATTTGTGAAATTTGTGATG(ps)CTATTG(ps)CTTTATT |
| Primer DNA | (SEQ ID NO.: 13) |

Primers for PCR and Sequencing

| | |
|---|---|
| PCR-F2 | GGGCGCCATCTAGAGAGTAA (SEQ ID NO.: 14) |
| PCR-R2 | GGGCGCCATTTATTTGTGAA (SEQ ID NO.: 15) |
| EGFP-473F | TGAATTACTTCCACGCCCCT (SEQ ID NO.: 16) |
| EGFP-1202F | GGAGTTTCCCCACACTGAGT (SEQ ID NO.: 17) |
| EGFP-1637F | CACATGAAGCAGCACGACTT (SEQ ID NO.: 18) |
| EGFP-1656R | AAGTCGTGCTGCTTCATGTG (SEQ ID NO.: 19) |
| EGFP-1236R | CTTCAGTCTCCACCCACTCA (SEQ ID NO.: 20) |
| EGFP-885R | GGCCAGCTTGAGACTACCC (SEQ ID NO.: 21) |
| EGFP-492R | AGGGGCGTGGAAGTAATTCA (SEQ ID NO.: 22) |

PCR Analysis: PCR was performed using primers PCR-F2 and PCR-R2. PCR products were resolved using a 1% agarose gel and purified using the QIAquick Gel Extraction Kit (Qiagen Catalog #28704). PCR using the no-RT controls as template showed no PCR product, indicating that the RNA samples were not contaminated with DNA. Table 22.

FIG. 25 depicts the results of the PCR from the RT reaction using WT RNA and Superscript Enzyme. Table 23. The 2.2 kb fragment of interest is present in reaction 1. 5 μl of the PCR reaction was loaded per lane.

TABLE 23

| | | | | | β- |
|---|---|---|---|---|---|
| | | RNA | Primer | | Thujaplicinol |
| Reaction | Sample | (μg) | for RT | Enzyme | (μM) |
| 1 | EGFP-WT | 1.5 | SV40 | Superscript | 0 |
| | | | DNA | | |
| 2 | EGFP-WT | 1.5 | PBS | Superscript | 0 |
| | | | DNA | | |

LM = Low DNA Mass Ladder (Invitrogen Catalog # 10068-013: 2 kb, 1.2 kb, 0.8 kb, 0.4 kb, 0.2 kb, 0.1 kb bands)

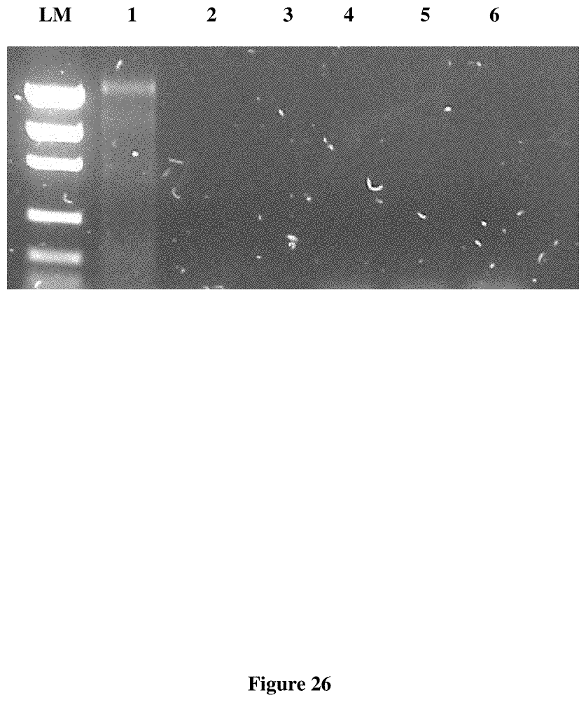

FIG. 26 depicts the results of the PCR from the no-RT control reaction using Superscript Enzyme. Table 24. The 2.2 kb fragment of interest is present in reaction 1 only, indicating no gDNA contamination. 5 μl PCR reaction is loaded per lane.

TABLE 24

| | | | | | β- |
|---|---|---|---|---|---|
| | | RNA | Primer | | Thujaplicinol |
| Reaction | Sample | (μg) | for RT | Enzyme | (μM) |
| 1 | EGFP-WT | 1.5 | SV40 | Superscript | 0 |
| | | | DNA | | |
| 2 | EGFP-WT | 1.5 | SV40 | — | 0 |
| | | | DNA | | |
| 3 | eGFP- | 1.5 | SV40 | — | 0 |
| | Alpha-Thio- | | DNA | | |
| | Uridine | | | | |
| 4 | eGFP- | 1.5 | SV40 | — | 0 |
| | 5MeC, | | DNA | | |
| | PseudoU | | | | |
| 5 | eGFP-2- | 1.5 | SV40 | — | 0 |
| | Thio-U | | DNA | | |
| 6 | PCR NTC | 0 | — | — | 0 |

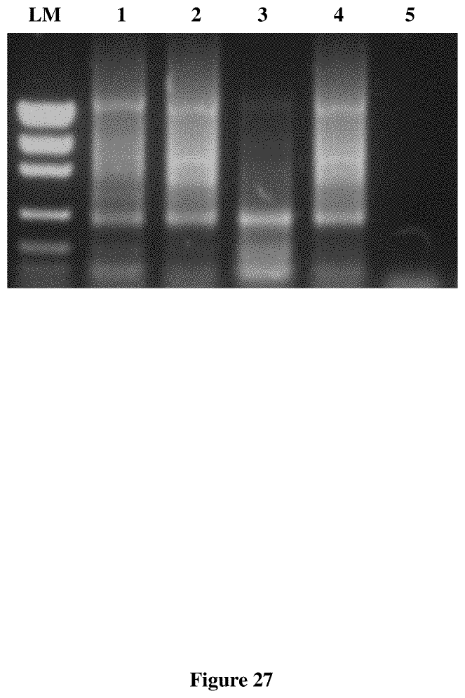

FIG. 27 depicts the results of the PCR from the RT reactions using Superscript Enzyme and SV40 DNA primer. Table 25. The 2.2 kb fragment of interest is present in all reactions. 5 μl PCR reaction was loaded per lane.

TABLE 25

| | | | | | β- |
|---|---|---|---|---|---|
| | | RNA | Primer | | Thujaplicinol |
| Reaction | Sample | (μg) | for RT | Enzyme | (μM) |
| 1 | EGFP-WT | 1.5 | SV40 | Superscript | 0 |
| | | | DNA | | |
| 2 | eGFP- | 1.5 | SV40 | Superscript | 0 |
| | Alpha-Thio- | | DNA | | |
| | Uridine | | | | |
| 3 | eGFP- | 1.5 | SV40 | Superscript | 0 |
| | 5MeC, | | DNA | | |
| | PseudoU | | | | |
| 4 | eGFP-2- | 1.5 | SV40 | Superscript | 0 |
| | Thio-U | | DNA | | |
| 5 | PCR NTC | 0 | — | — | 0 |

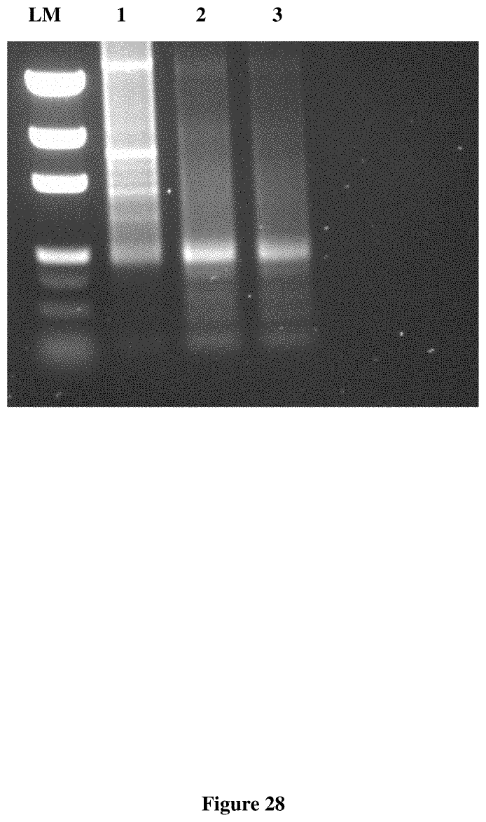

FIG. 28 depicts the results of the PCR from the RT reaction using WT RNA and Superscript Enzyme. Table 26. The 2.2 kb fragment of interest present in reaction all reactions. 5 μl PCR reaction was loaded per lane.

TABLE 26

| | | | | | β- |
|---|---|---|---|---|---|
| | | RNA | Primer | | Thujaplicinol |
| Reaction | Sample | (μg) | for RT | Enzyme | (μM) |
| 1 | EGFP-WT | 1.5 | SV40 | Superscript | 0 |
| | | | DNA | | |
| 2 | EGFP-WT | 1.5 | PBS | Superscript | 0 |
| | | | RNA | | |
| 3 | EGFP-WT | 1.5 | PCR-R2 | Superscript | 0 |

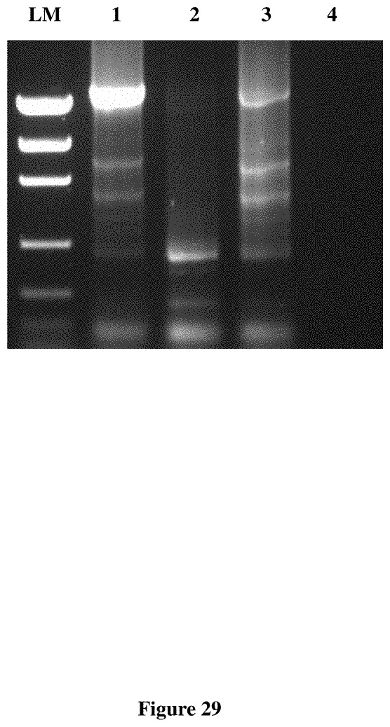

FIG. 29 depicts the results of the PCR from the RT reaction using Superscript enzyme and SV40 RNA primer. Table 27. The 2.2 kb fragment of interest is present in all reactions. 5 μl PCR reaction was loaded per lane.

TABLE 27

| | | | | | β- |
|---|---|---|---|---|---|
| | | RNA | Primer | | Thujaplicinol |
| Reaction | Sample | (μg) | for RT | Enzyme | (μM) |
| 1 | eGFP- | 1.5 | SV40 | Superscript | 0 |
| | Alpha-Thio- | | RNA | | |
| | Uridine | | | | |
| 2 | eGFP- | 1.5 | SV40 | Superscript | 0 |
| | 5MeC, | | RNA | | |
| | PseudoU | | | | |
| 3 | eGFP-2- | 1.5 | SV40 | Superscript | 0 |
| | Thio-U | | RNA | | |
| 4 | EGFP-WT | 1.5 | SV40 | — | 0 |
| | | | RNA | | |

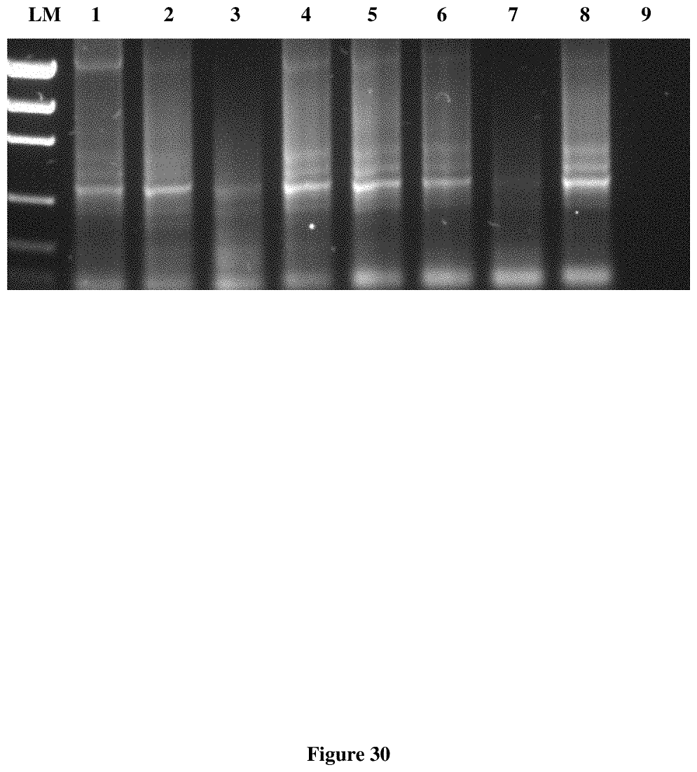

FIG. 30 depicts the results of the PCR from the RT reaction using HIV RT and SV40 DNA and RNA primers. Table 28. The 2.2 kb fragment of interest present in reactions 1, 2, 4, 5, 6, and 8. 5 μl PCR reaction was loaded per lane.

TABLE 28

| | | | | | β- |
|---|---|---|---|---|---|
| | | RNA | Primer | | Thujaplicinol |
| Reaction | Sample | (μg) | for RT | Enzyme | (μM) |
| 1 | EGFP-WT | 1.5 | SV40 | HIV RT | 0 |
| | | | DNA | | |
| 2 | eGFP- | 1.5 | SV40 | HIV RT | 0 |
| | Alpha-Thio- | | DNA | | |
| | Uridine | | | | |
| 3 | eGFP- | 1.5 | SV40 | HIV RT | 0 |
| | 5MeC, | | DNA | | |
| | PseudoU | | | | |
| 4 | eGFP-2- | 1.5 | SV40 | HIV RT | 0 |
| | Thio-U | | DNA | | |
| 5 | EGFP-WT | 1.5 | SV40 | HIV RT | 0 |
| | | | RNA | | |
| 6 | eGFP- | 1.5 | SV40 | HIV RT | 0 |
| | Alpha-Thio- | | RNA | | |
| | Uridine | | | | |
| 7 | eGFP- | 1.5 | SV40 | HIV RT | 0 |
| | 5MeC, | | RNA | | |
| | PseudoU | | | | |
| 8 | eGFP-2- | 1.5 | SV40 | HIV RT | 0 |
| | Thio-U | | RNA | | |
| 9 | PCR NTC | 0 | — | — | 0 |

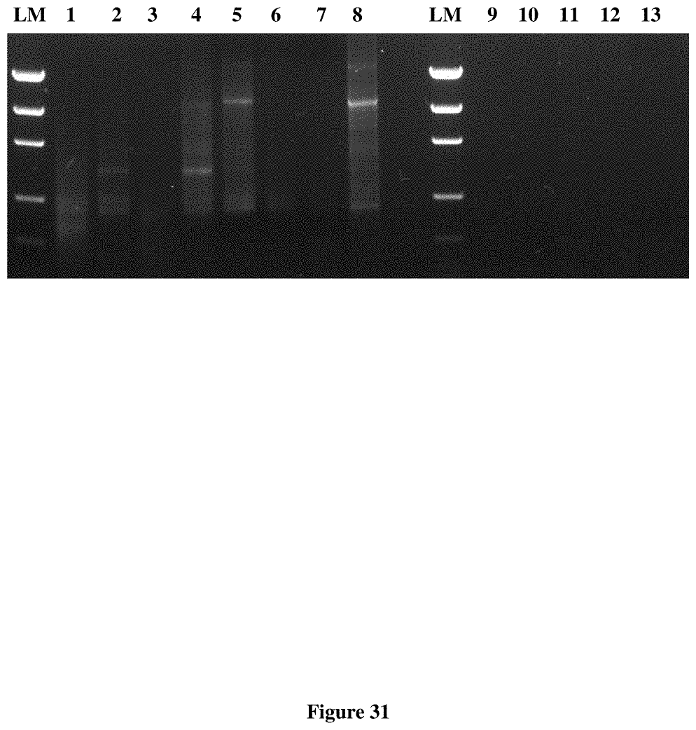

FIG. 31 depicts the results from the PCR from the RT reaction using HIV RT and SV40 DNA and RNA primers plus β-Thujaplicinol. Table 29. The 2.2 kb fragment of interest is present in reactions 4, 5, and 8. 5 μl PCR reaction was loaded per lane.

TABLE 29

| | | | | | β- |
|---|---|---|---|---|---|
| | | RNA | Primer | | Thujaplicinol |
| Reaction | Sample | (μg) | for RT | Enzyme | (μM) |
| 1 | EGFP-WT | 1.5 | SV40 | HIV RT | 0.2 |
| | | | DNA | | |
| 2 | eGFP- | 1.5 | SV40 | HIV RT | 0.2 |
| | Alpha-Thio- | | DNA | | |
| | Uridine | | | | |
| 3 | eGFP- | 1.5 | SV40 | HIV RT | 0.2 |
| | 5MeC, | | DNA | | |
| | PseudoU | | | | |
| 4 | eGFP-2- | 1.5 | SV40 | HIV RT | 0.2 |
| | Thio-U | | DNA | | |
| 5 | EGFP-WT | 1.5 | SV40 | HIV RT | 0.2 |
| | | | RNA | | |
| 6 | eGFP- | 1.5 | SV40 | HIV RT | 0.2 |
| | Alpha-Thio- | | RNA | | |
| | Uridine | | | | |
| 7 | eGFP- | 1.5 | SV40 | HIV RT | 0.2 |
| | 5MeC, | | RNA | | |
| | PseudoU | | | | |
| 8 | eGFP-2- | 1.5 | SV40 | HIV RT | 0.2 |
| | Thio-U | | RNA | | |
| 9 | EGFP-WT | 1.5 | SV40 | — | 0.2 |
| | | | DNA | | |
| 10 | eGFP- | 1.5 | SV40 | — | 0.2 |
| | Alpha-Thio- | | DNA | | |
| | Uridine | | | | |
| 11 | eGFP- | 1.5 | SV40 | — | 0.2 |
| | 5MeC, | | DNA | | |
| | PseudoU | | | | |
| 12 | eGFP-2- | 1.5 | SV40 | — | 0.2 |
| | Thio-U | | DNA | | |
| 13 | PCR NTC | 0 | — | — | 0 |

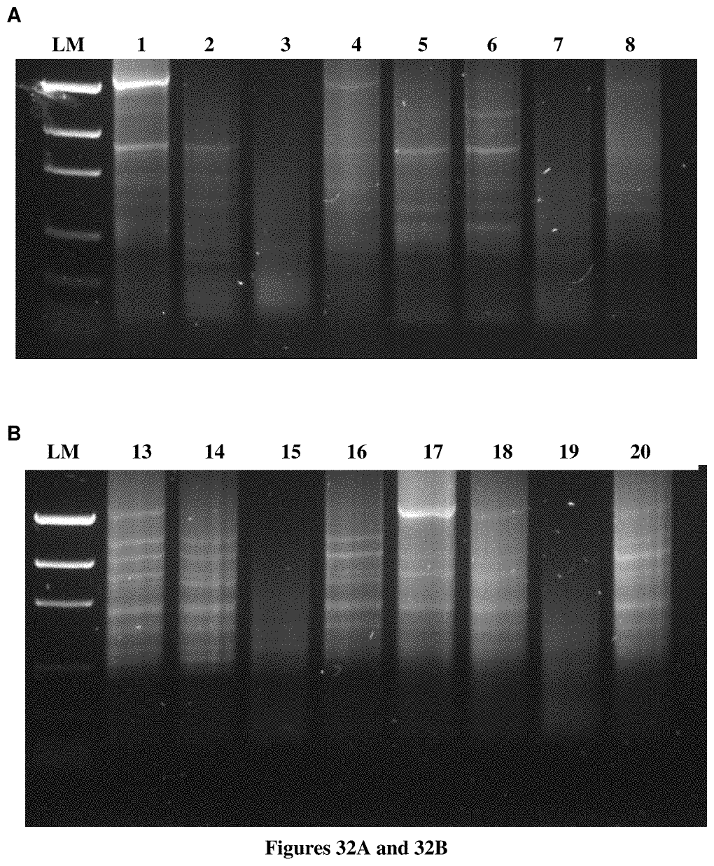

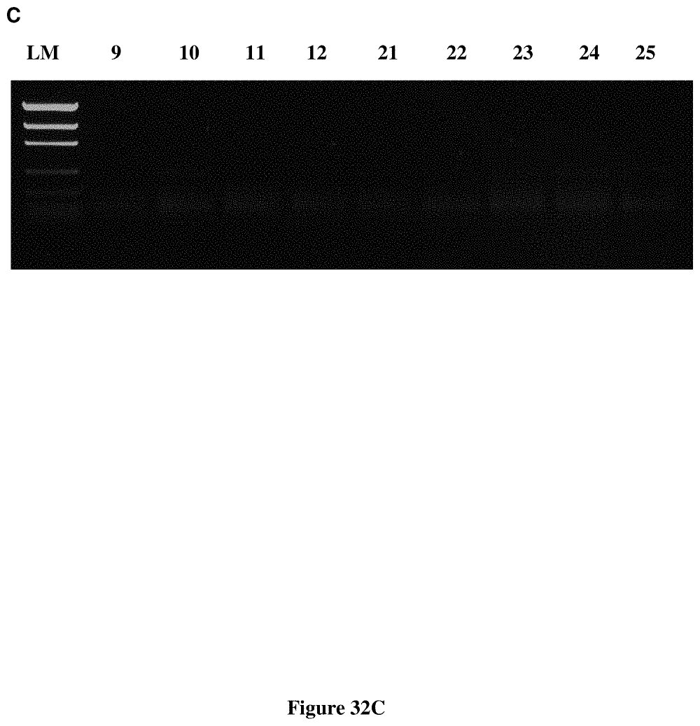

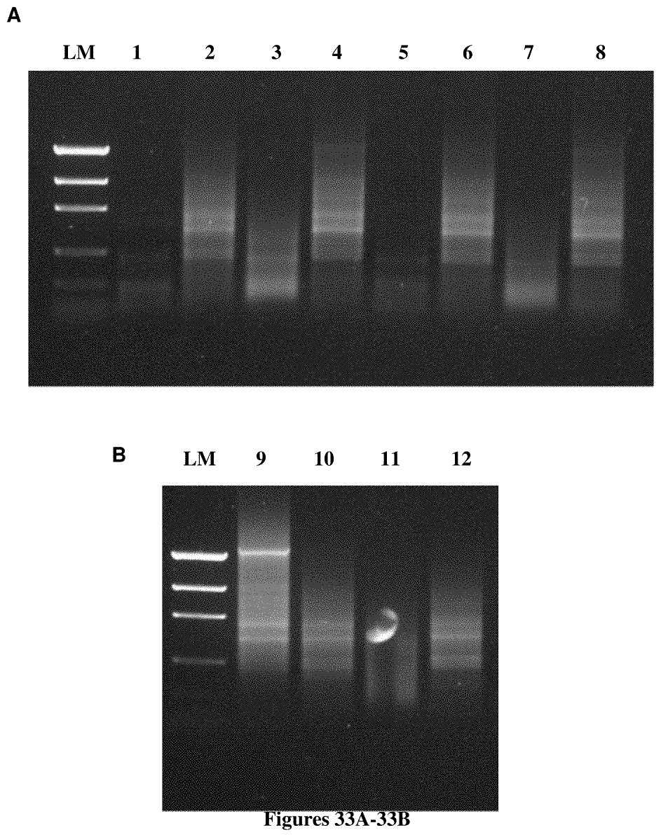

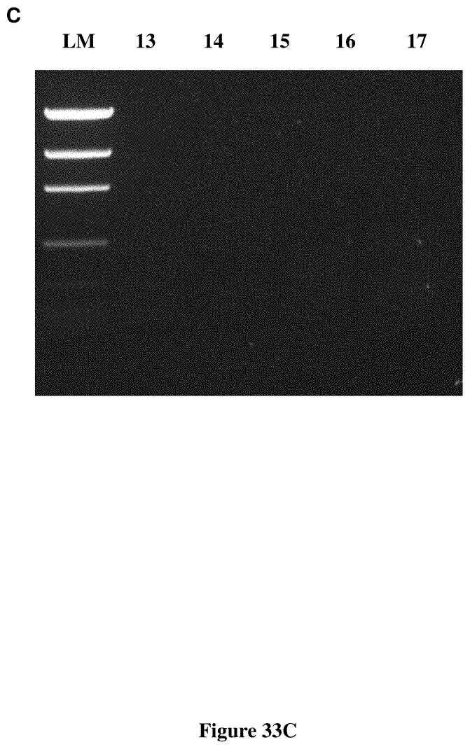

FIGS. 32A-33C depicts the results of the PCR from the RT reaction using HIV RT, SV40 DNA, and RNA primers plus β-Thujaplicinol in designated reactions. Table 30. The 2.2 kb fragment of interest is present in reactions 1, 4, 8, 13, 17, 18 and 20. 20 μl PCR reaction was loaded per lane.

TABLE 30

| | | | | | β- |
|---|---|---|---|---|---|
| | | RNA | Primer | | Thujaplicinol |
| Reaction | Sample | (μg) | for RT | Enzyme | (μM) |
| 1 | EGFP-WT | 2 | SV40 | HIV RT | 0 |
| | | | DNA | | |
| 2 | eGFP- | 2 | SV40 | HIV RT | 0 |
| | Alpha-Thio- | | DNA | | |
| | Uridine | | | | |
| 3 | eGFP- | 2 | SV40 | HIV RT | 0 |
| | 5MeC, | | DNA | | |
| | PseudoU | | | | |
| 4 | eGFP-2- | 2 | SV40 | HIV RT | 0 |
| | Thio-U | | DNA | | |
| 5 | EGFP-WT | 2 | SV40 | HIV RT | 0 |
| | | | RNA | | |
| 6 | eGFP- | 2 | SV40 | HIV RT | 0 |
| | Alpha-Thio- | | RNA | | |
| | Uridine | | | | |
| 7 | eGFP- | 2 | SV40 | HIV RT | 0 |
| | 5MeC, | | RNA | | |
| | PseudoU | | | | |
| 8 | eGFP-2- | 2 | SV40 | HIV RT | 0 |
| | Thio-U | | RNA | | |
| 9 | EGFP-WT | 2 | SV40 | — | 0 |
| | | | DNA | | |
| 10 | eGFP- | 2 | SV40 | — | 0 |
| | Alpha-Thio- | | DNA | | |
| | Uridine | | | | |
| 11 | eGFP- | 2 | SV40 | — | 0 |
| | 5MeC, | | DNA | | |
| | PseudoU | | | | |
| 12 | eGFP-2- | 2 | SV40 | — | 0 |
| | Thio-U | | DNA | | |
| 13 | EGFP-WT | 2 | SV40 | HIV RT | 1 |
| | | | DNA | | |
| 14 | eGFP- | 2 | SV40 | HIV RT | 1 |
| | Alpha-Thio- | | DNA | | |
| | Uridine | | | | |
| 15 | eGFP- | 2 | SV40 | HIV RT | 1 |
| | 5MeC, | | DNA | | |
| | PseudoU | | | | |
| 16 | eGFP-2- | 2 | SV40 | HIV RT | 1 |
| | Thio-U | | DNA | | |
| 17 | EGFP-WT | 2 | SV40 | HIV RT | 1 |
| | | | RNA | | |
| 18 | eGFP- | 2 | SV40 | HIV RT | 1 |
| | Alpha-Thio- | | RNA | | |
| | Uridine | | | | |
| 19 | eGFP- | 2 | SV40 | HIV RT | 1 |
| | 5MeC, | | RNA | | |
| | PseudoU | | | | |
| 20 | eGFP-2- | 2 | SV40 | HIV RT | 1 |
| | Thio-U | | RNA | | |
| 21 | EGFP-WT | 2 | SV40 | — | 1 |
| | | | RNA | | |
| 22 | eGFP- | 2 | SV40 | — | 1 |
| | Alpha-Thio- | | DNA | | |
| | Uridine | | | | |
| 23 | eGFP- | 2 | SV40 | — | 1 |
| | 5MeC, | | DNA | | |
| | PseudoU | | | | |
| 24 | eGFP-2- | 2 | SV40 | — | 1 |
| | Thio-U | | DNA | | |
| 25 | PCR NTC | 0 | — | — | 1 |

FIG. 33A-33C depict the results of the PCR from RT reaction using HIV RT and Phosphorothioate Primer DNA plus β-Thujaplicinol in designated reactions. Table 31. The 2.2 kb fragment of interest is present in reactions 4, 6, and 9. 20 μl PCR reaction loaded per lane.

TABLE 31

| | | | | | β- |
|---|---|---|---|---|---|
| | | RNA | Primer | | Thujaplicinol |
| Reaction | Sample | (μg) | for RT | Enzyme | (μM) |
| 1 | EGFP-WT | 5 | Phospho- | HIV RT | 0 |
| | | | rothioate | | |
| | | | Primer DNA | | |
| 2 | eGFP- | 5 | Phospho- | HIV RT | 0 |
| | Alpha-Thio- | | rothioate | | |
| | Uridine | | Primer DNA | | |
| 3 | eGFP- | 5 | Phospho- | HIV RT | 0 |
| | 5MeC, | | rothioate | | |
| | PseudoU | | Primer DNA | | |
| 4 | eGFP-2- | 5 | Phospho- | HIV RT | 0 |
| | Thio-U | | rothioate | | |
| | | | Primer DNA | | |
| 5 | EGFP-WT | 5 | Phospho- | HIV RT | 0.2 |
| | | | rothioate | | |
| | | | Primer DNA | | |
| 6 | eGFP- | 5 | Phospho- | HIV RT | 0.2 |
| | Alpha-Thio- | | rothioate | | |
| | Uridine | | Primer DNA | | |
| 7 | eGFP- | 5 | Phospho- | HIV RT | 0.2 |
| | 5MeC, | | rothioate | | |
| | PseudoU | | Primer DNA | | |
| 8 | eGFP-2- | 5 | Phospho- | HIV RT | 0.2 |
| | Thio-U | | rothioate | | |
| | | | Primer DNA | | |
| 9 | EGFP-WT | 5 | SV40 | HIV RT | 0.2 |
| | | | DNA | | |
| 10 | eGFP- | 5 | SV40 | HIV RT | 0.2 |
| | Alpha-Thio- | | DNA | | |
| | Uridine | | | | |
| 11 | eGFP- | 5 | SV40 | HIV RT | 0.2 |
| | 5MeC, | | DNA | | |
| | PseudoU | | | | |
| 12 | eGFP-2- | 5 | SV40 | HIV RT | 0.2 |
| | Thio-U | | DNA | | |
| 13 | EGFP-WT | 5 | Phospho- | — | 0.2 |
| | | | rothioate | | |
| | | | Primer DNA | | |
| 14 | eGFP- | 5 | Phospho- | — | 0.2 |
| | Alpha-Thio- | | rothioate | | |
| | Uridine | | Primer DNA | | |
| 15 | eGFP- | 5 | Phospho- | — | 0.2 |
| | 5MeC, | | rothioate | | |
| | PseudoU | | Primer DNA | | |
| 16 | eGFP-2- | 5 | Phospho- | — | 0.2 |
| | Thio-U | | rothioate | | |
| | | | Primer DNA | | |
| 17 | PCR NTC | 0 | — | — | 0 |

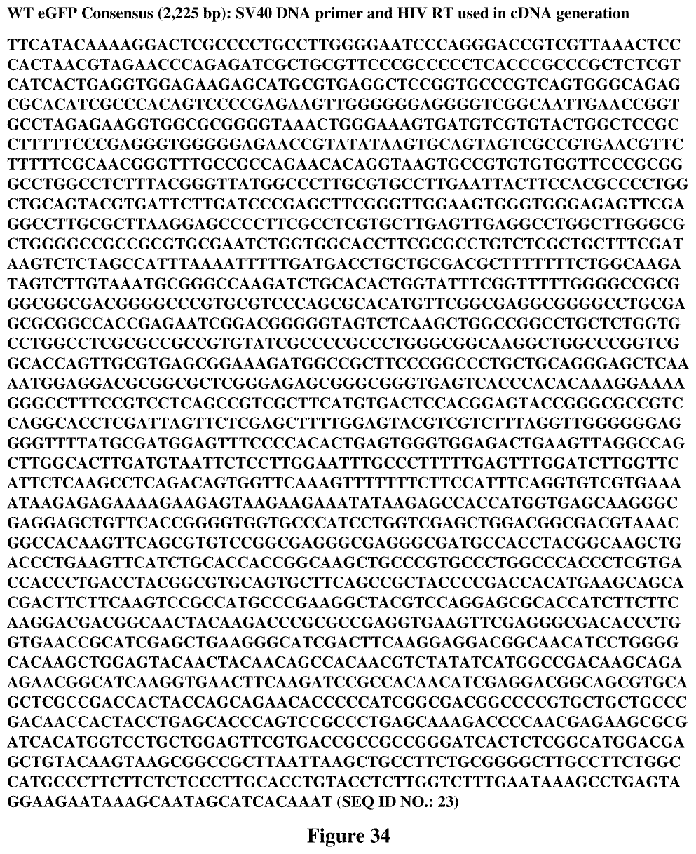

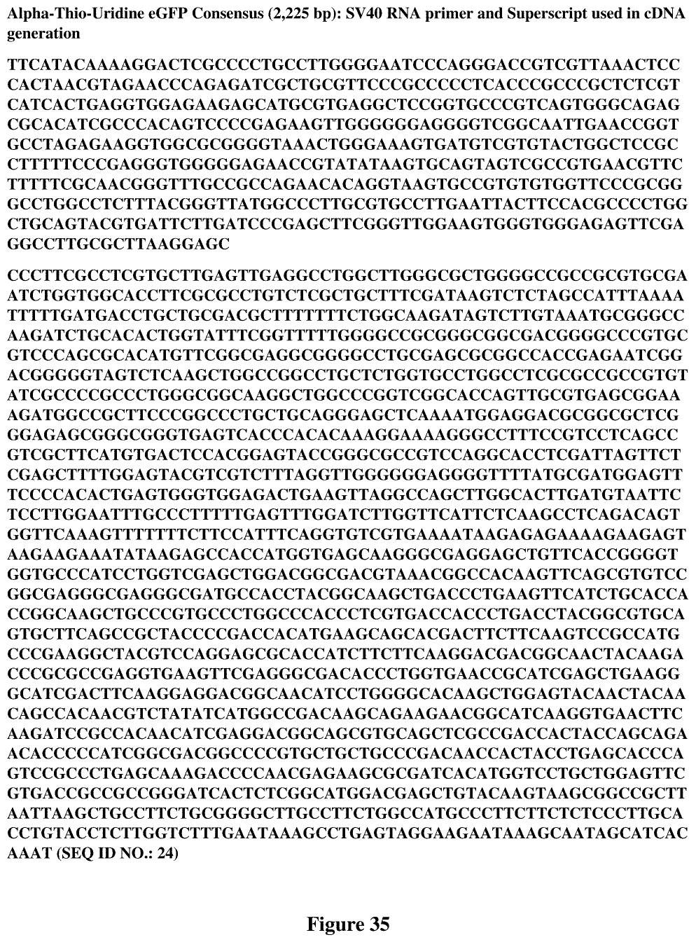

Sequence Analysis: Sequencing was performed using BigDye Terminator Cycle Sequencing. Data analysis was performed by GENEWIZ with DNASTAR Lasergene software. FIG. 34 (SEQ ID NO.: 23) depicts the WT eGFP Consensus sequence (2,225 bp); SV40 DNA primer and HIV RT used in cDNA generation. FIG. 35 (SEQ ID NO.: 24) depicts the Alpha-Thio-Uridine eGFP Consensus (2,225 bp); SV40 RNA primer and Superscript used in cDNA generation.

Results: The WT eGFP (SV40 DNA primer and HIV RT) consensus sequence is a 100% match to the reference sequence. The Alpha-Thio-Uridine eGFP (SV40 RNA primer and Superscript) consensus sequence is a 100% match to the reference sequence.

### Example 8

The modulation of the expression of a fluorescent gene reporter by HIV reverse transcriptase (RT) ribonuclease H (RNase H) activity upon treatment with antisense DNA oligonucleotides (approximately 20-30 bases) capable of hybridizing with the mRNA of the reporter was evaluated.

The protocol was designed:

Step 1: Generation of a mouse glioma GL261 cell clone stably expressing the HIV RT with intact RNase H activity.

Step 1B: Preparation and validation of the experimental model to be used in Step 2.

Step 1B1. Assess the functional dimerization of the two HIV Reverse Transcriptase subunits in the p51/p66 stable cell clones developed in the first step.

Step 1B2. Remove the potential plasmidic contamination of the mRNA solutions prepared by TriLink®.

Step 1. Generation of a mouse glioma GL261 cell clone stably expressing the HIV RT with intact RNase H activity.

Step 1B: Preparation and validation of the experimental model

### Experimental Model:

Cell lines utilized were 1) wild type model: mouse GL261 glioma cells, 2) a HIV RT expressing model: mouse GL261 glioma cells stably transfected both with the p51/pD2539-CAG and the p66/pD2533 plasmids (clone 6 and 7), and 3) a negative control cell line: mouse GL261 glioma cells stably transfected with the p51/pD2539-CAG plasmid (clone 1).

Culture conditions utilized are 1) wild type model: DMEM+10% FBS, 2) HIV RT-expressing model: DMEM+10% FBS+G418 (500 μg/mL)+Puromycin (2 μg/mL), and 3) Negative control cell line: DMEM+10% FBS+G418 (500 μg/mL).

The experimental procedure and assay readout for analysis of the dimerization of the two HIV RT subunits was as follows: 1) the different cellular models were thawed and cultured in their respective culture media. 2) cells were collected by trypsinization, 3) whole cell extracts were prepared and resolved on a polyacrylamide gel in native conditions, 4) the gel was stained with Coomassie blue, 5) the patterns between the wild type cell model and the stable cell clones (mono and doubly transfected) were compared in order to identify the bands corresponding to the p51, p66 proteins and to the p51/p66 heterodimer 6) another gel was run in the same conditions before being reduced and denatured, 7) the proteins were blotted on a PVDF membrane on which the hybridization of the p51/p66 polyclonal antibody was tested.

The experimental procedure and assay readout for the analysis and removal of the mRNA contamination by plasmidic DNA was as follows: 1) the 4 mRNAs solutions were thawed, aliquoted and stored at −80° C., 2) aliquots of the mRNAs solutions were treated with the DNase as recommended by the manufacturer (Qiagen, cat #79254): four rounds of DNase were successively performed, 3) following the DNase digestion, the mRNAs were purified on columns as recommended by the manufacturer (Qiagen, RNeasy Elute), 4) the eluates were quantified and controlled on an Agarose gel, 5) a PCR amplification reaction was performed to evaluate the DNA contamination of the mRNA solutions before and after the DNase treatment.

Timeline of p51/p66 dimerization. Different cell models are thawed and cultured in specific culture media. The native whole cell extracts are prepared. Following preparation, a native polyA gel is run and stained with Coomassie blue. In addition, a native polyA gel is run, blotted, and the polyclonal p51/p66 antibody is tested.

Timeline of mRNAs solutions control and DNA digestion. mRNA solutions are thawed and digested with DNase. The solutions are purified on a column. The mRNA is quantified and quality control on an agarose gel is performed after purification. A PCR reaction of the mRNA aliquots is performed on aliquots from before and after purification.

Results: p51/p66 dimerization: whole cell extracts staining from Native PAGE. FIG. 36. The level of expression of the p51 and p66 proteins did not allow to detect their monomer nor their heterodimers among the whole cell extracts from the different tested clones.

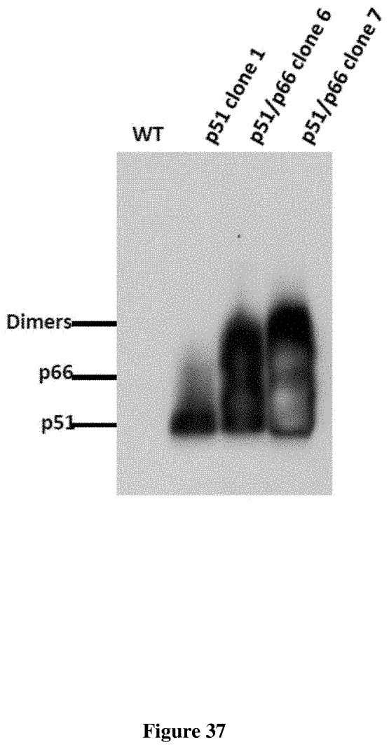

Results: p51/p66 dimerization: anti p51/p66 Western blotting from Native PAGE. FIG. 37. The Western blotting performed with the specific antibody against the two subunits of the HIV Reverse transcriptase on a native PAGE experiment showed that 1) p51 in Clone 1 stably transfected with the p51 construct and 2) a low level of p51, p66 and more pronounced level of proteins with molecular weights corresponding to the dimers of the two subunits in the clones 6 and 7 stably co-transfected with the p51 and p66 constructs. The extracts from the wild type GL-261 cell line did not reveal a specific signal.

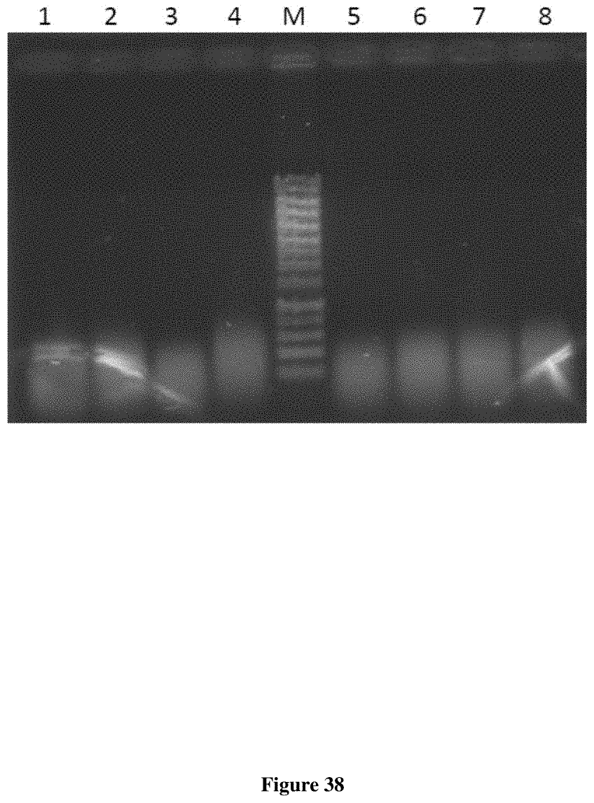

Results of DNA digestion: PCR before and after DNase treatment are depicted in FIG. 38. M=marker. The samples are indicated in Table 32. No PCR product could be detected from the reactions performed with the different mRNA solutions proceeded or not with the DNase digestion and the subsequent column purification.

TABLE 32

| | Well number | |
|---|---|---|
| eGFP Transcript | Before DNase | After DNase |
| Wild type | 1 | 2 |
| Alpha-Thio-Uridine pre-DNase treatment | 3 | 4 |
| 2-Thio-U pre-DNase treatment | 5 | 6 |
| 5meC, PseudoU pre-DNase treatment | 7 | 8 |

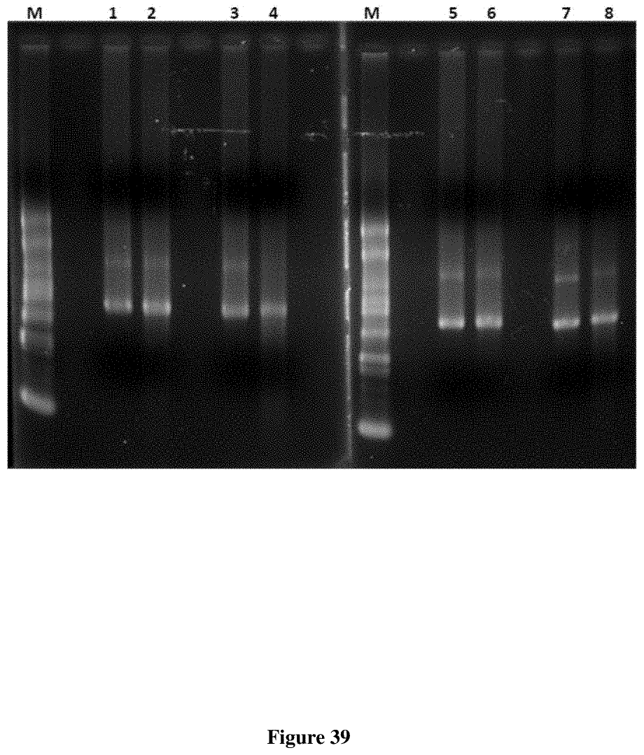

Results of DNA digestion: mRNA quality check before and after DNase treatment. FIG. 39. M=marker. The samples are indicated in Table 33. The non-denaturating conditions of the agarose gel electrophoresis did not allow to obtain a single band for each of the tested mRNA. The comparison of the migration pattern before and after DNase treatment revealed however that the DNA digestion did not degrade the mRNAs.

TABLE 33

| | Well number | |
|---|---|---|
| eGFP Transcript | Before DNase | After DNase |
| Wild type | 1 | 5 |
| Alpha-Thio-Uridine pre-DNase treatment | 2 | 6 |
| 2-Thio-U pre-DNase treatment | 3 | 7 |
| 5meC, PseudoU pre-DNase treatment | 4 | 8 |

Dimerization of the p51 and p66 subunits in the clones established in Step 1: The western blot analysis of p51 and p66 expression performed with denaturating conditions revealed an intense expression level of the two subunits in the stably co-transfected clone 6 and 7. The native conditions applied in showed a much lower level of the p51 and p66 monomers compared to the level of the dimeric proteins in those clones, while the p51 subunit is still expressed at a high level in the stably mono-transfected clone 1. This result indicates that in the stably co-transfected clone 6 and 7, p51 and p66 subunits are mostly involved in the protein dimers.

mRNAs purification: The PCR did not allow detection of potential DNA contamination of the mRNAs solutions. The agarose gel electrophoresis and the measure of UV absorbance (A260/A280) of the mRNA solutions demonstrate that the DNase treatment followed by the column purification of mRNAs did not alter their integrity. Table 34.

TABLE 34

OD 260/280

| Sample | Pre-treatment | Post-treatment |
|---|---|---|
| Wild type | 2.07 | 2.05 |
| Alpha-Thio-Uridine | 2.06 | 2.06 |
| 2-Thio-U | 2.07 | 2.06 |
| 5meC, PseudoU | 1.72 | 1.71 |

### Example 9

Step 2: Transfection of parental and HIV RT-expressing glioma GL261 cells with WT GFP RNA and oligonucleotides and GFP expression analysis.

### Experimental Model:

The cell lines that will be utilized are 1) wild type model: mouse GL261 glioma cells and 2) HIV RT expressing model: mouse GL261 glioma cells stably transfected with the p51/pD2539-CAG and the p66/pD2533 plasmids (clone 7).

The culture conditions that will be utilized are 1) wild type model: DMEM+10% FBS and 2) HIV RT expressing model: DMEM+10% FBS+G418 (500 μg/mL)+Puromycin (2 μg/mL).

The two cell lines to be tested (WT GL261, Clone 7). The number of oligonucleotides is two. The concentration of the oligonucleotide is 50 ng. The number of mRNAs is one (WT GFP mRNA reporter gene). The concentrations of the mRNA are 200 ng and 500 ng. The time points are 8 h, 24 h, 72 h, and 7 days (dependent upon the cell density and viability after one week of culture).

Transfection samples are 1. Zero; 2. WT RNA; 3. WT RNA annealed with Oligo; 4. WT RNA annealed with Oligo 2; 5. WT RNA mixed with Oligo 1; 6. WT RNA mixed with Oligo 2; 7. WT RNA annealed with Oligo 1 then mixed with Oligo 2; 8. WT RNA annealed with Oligo 2 then mixed with Oligo 1; 9. WT RNA mixed with Oligo 1 and Oligo 2; and 10. WT RNA mixed with Oligo 2 and Oligo 1.

The experimental procedure to be followed is 1) cells will be seeded in 96 well plates; 2) 24 h later, cells will be transfected using the Viromer red transfection reagent (Lipocalyx) with the different conditions to be tested; and 3) the expression of the GFP reporter gene will be kinetically measured on a kinetic high content imaging platform (Incucyte, Essen Bioscience).

The protocols for the RNA/Oligonucleotide(s)/Viromer preparation is as follows: Conditions 1 and 2: RNA/Viromer red (2) or transfection buffer/Viromer red complexes will be prepared as recommended by the manufacturer (the different type of nucleotides will be added to diluted Viromer and mixed by pipetting) and the complexes will be added to the cells. Conditions 3 and 4: RNA and oligonucleotide will be diluted at their respective concentrations to be tested and mixed in the transfection buffer after which the mixture will be placed at 95 C for 2 min and will be cooled down on the bench for approximately 1 h. The products of the annealing reaction will be complexed to Viromer red as recommended by the manufacturer (the different type of nucleotides will be added to diluted Viromer and mixed by pipetting) and the complexes will be added to the cells. Conditions 5 and 6: RNA and oligonucleotide will be diluted at their respective concentrations to be tested and mixed in the transfection buffer. The mixture will be complexed to Viromer red as recommended by the manufacturer (the different type of nucleotides will be added to diluted Viromer and mixed by pipetting) and the complexes will be added to the cells. Conditions 7 and 8: RNA and oligonucleotide 1 (condition 7) or 2 (condition 8) will be diluted at their respective concentrations to be tested and mixed in the transfection buffer. The mixture will be placed at 95 C for 2 min and will be cooled down on the bench for approximately 1 h. The products of the annealing reaction will be mixed with the oligonucleotide 2 (condition 7) or 1 (condition 8) diluted at the concentration to be tested in the transfection buffer. The mixture will be complexed to Viromer red as recommended by the manufacturer (the different type of nucleotides will be added to diluted Viromer and mixed by pipetting) and the complexes will be added to the cells. Conditions 9 and 10: RNA and oligonucleotide 1 (condition 9) or 2 (condition 10) will be diluted at their respective concentrations to be tested and mixed in the transfection buffer. The products of the first mixture will be mixed with the oligonucleotide 2 (condition 9) or 1 (condition 10) diluted at their concentration to be tested in the transfection buffer. The mixture will be complexed to Viromer red as recommended by the manufacturer (the different type of nucleotides will be added to diluted Viromer and mixed by pipetting) and the complexes will be added to the cells. Complex formation will be allowed for 15 minutes before adding to the following to the cells: 10 μL for 100 ng mRNA, 20 μL for 200 ng mRNA and 50 μL for 500 ng mRNA, added on 100 μL growth medium. The fluorescence intensity for each tested condition will be measured at each time point to be tested on a kinetic high content imaging platform (Incucyte, Essen Bioscience).

### Example 10

The GL261 Clone 7 cell line transfected with reverse transcriptase has reverse transcriptase activity above the background noise seen in the wildtype GL261 clone.

Radioactive RT Assay: Cell lysates from GL261 wild type or Clone 7 cells were prepared according to the method described in Ansari-Lari and Gibbs (1994). Extracts were assayed in a 25 uL RT reaction containing 20 mM Tris (pH 8.3), 100 mM KCl, 5 mM MgCl₂, 0.3 mM glutathione, and containing 2.5 U/mL of poly(rA)-dT12-18 and 1 μCi of ³H-TTP (20 Ci/mmol). Reactions were incubated for 20 minutes at 37° C. and stopped by the addition of 175 μL of ice cold 10% TCA. Nucleic acids were precipitated to 20 minutes on ice. Precipitated reactions were transferred to a 96 well glass fiber filter plate and a vacuum was applied. Wells were washed two times with 250 μL of ice cold 10% TCA and once with ice cold 100% ethanol. The filter plate was allowed to dry and then 30 uL of MicroScint O was added to each well. Wells were counted for 1 minute each on a TopCount scintillation counter. Raw counts per minute were graphed for each reaction.

In this experiment, the amount of cell extract was titrated down in three fold increments starting with 5 μL of extract. Negative controls were included in which no extract was added. Additional negative controls contained 5 μL of each extract but lacking the poly(rA)-dT12-18 template. Lastly, a positive control was included in which 50 Units of Multiscribe RT were included in the reaction. Triplicate reactions were performed for each condition. Table 35.

TABLE 35

| μL | | | | | | | | | | No Template | | | No Template | | |
|---|---|---|---|---|---|---|---|---|---|---|---|---|---|---|---|
| Extract | WT Extract | | | Clone 7 | | | Multiscribe RT | | | 5 μL WT | | | 5 μL Clone 7 | | |
| 5.000 | 628.4 | 533.0 | 477.6 | 3135.5 | 3159.7 | 3319.2 | 912.2 | 1307.0 | 1531.5 | 36.2 | 30.3 | 40.7 | 40.3 | 54.3 | 37.3 |
| 1.667 | 338.2 | 323.3 | 331.5 | 3671.2 | 3642.1 | 3698.6 | | | | | | | | | |
| 0.556 | 157 | 145.5 | 165.1 | 2360.1 | 2168.7 | 2218.3 | | | | | | | | | |
| 0.185 | 41.3 | 49.2 | 66.3 | 636.1 | 498.5 | 496.5 | | | | | | | | | |
| 0.062 | 20.6 | 103.5 | 39.2 | 51.4 | 29.2 | 96.1 | | | | | | | | | |
| 0 | 19.3 | 31.2 | 25.1 | 28.0 | 26.5 | 17.4 | | | | | | | | | |

Raw CPMs are presented.

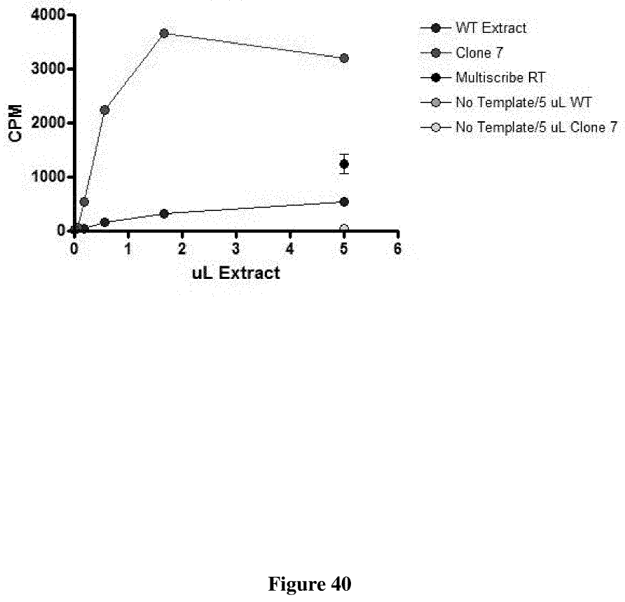

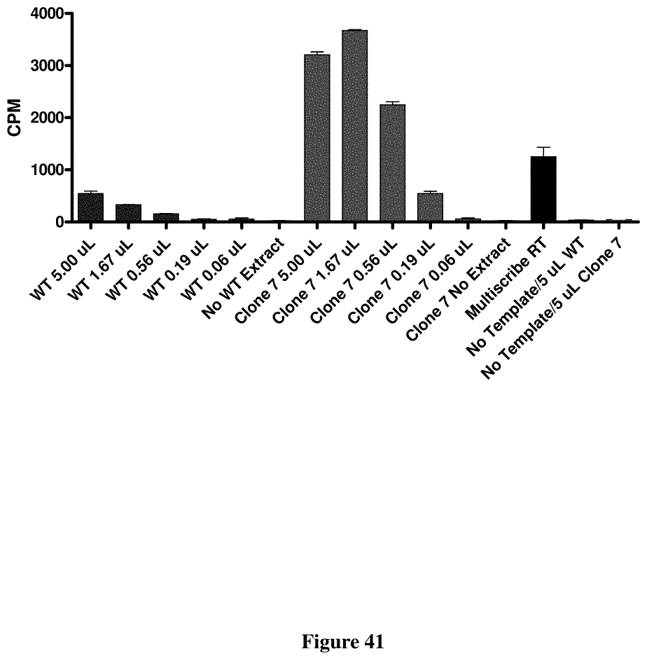

FIG. 40 depicts reverse transcriptase activity of cell extracts. Average CPMs were plotted as a function of extract volume included in the reaction. FIG. 41 depicts reverse transcriptase activity of cell extracts. Average CPMs for each reaction conditions are presented in bar graph format. Error bars represent standard errors of the mean. The GL261 Clone 7 cell line transfected with reverse transcriptase has reverse transcriptase activity above the background noise seen in the wildtype GL261 clone.

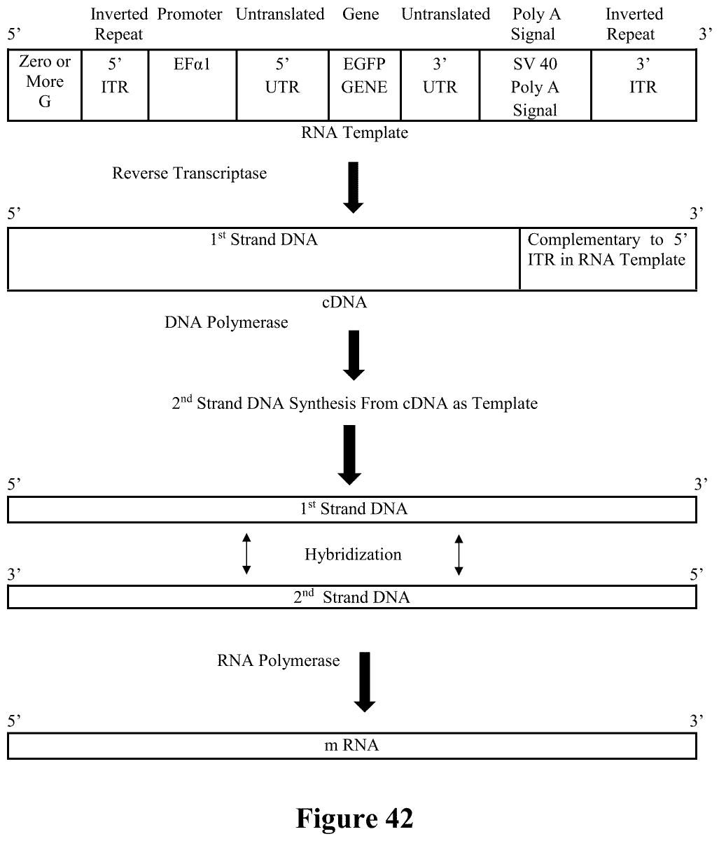

FIG. 42 illustrates another embodiment of an aviral RNA template for treating cells expressing reverse transcriptase. This figure illustrates sequences corresponding to a 5′ primer or inverted terminal repeat (ITR) sequence, a promoter sequence, a 5′ UTR sequence, a gene sequence, a 3′ UTR sequence, a poly A sequence, and a 3′ primer or ITR sequence. In various embodiments, additional nucleic acid sequences could be formed on the 5′ end of the template, for example a varying number of guanine nucleobases to assist in DNA formation, or even additional non-obligatory sequences which have no functional purpose or purpose related to expression of the gene reflected in the RNA template. Likewise, there could also be non-obligatory sequences between one or more of (i) the 5′ ITR and promoter sequences, (ii) the promoter and 5′ UTR sequences, (iii) the 5′ UTR and gene sequences, (iv) the gene and 3′ UTR sequences, (v) the 3′ UTR and Poly A Signal sequences, and (vi) the Poly A Signal and 3′ ITR sequences. However, there generally will be no non-obligatory sequences beyond the 3′ ITR sequence.

An inverted repeat (or IR) is a single stranded sequence of nucleotides followed downstream by its reverse complement. The intervening sequence of nucleotides between the initial sequence and the reverse complement can be any length including zero. When the intervening length is zero, the composite sequence is a palindromic sequence. For example, 5′-TTACGnnnnnnCGTAA-3′ is an inverted repeat sequence. It may be seen in this example that the IR sequences includes a first set of nucleotides (TTACG) which are the reverse complement of a second set of nucleotides (CGTAA). In other examples, the first set of nucleotides need not be 100% the reverse complement of the second set, but could be any percentage between at least 60% and at least 99% the reverse complement of the second set. An IR at the 5′ or 3′ end of an RNA or DNA segment is often referred to as an inverted terminal repeat (or ITR). An inverted terminal repeat sequence upon hybridization between the initial sequence and the downstream reverse complementary sequences causes the single strand polynucleotide to snap hack on itself and act as a primer for the synthesis of DNA. without the need to add a separate primer(i.e., a primer which is not part of the RNA template).

In one example of the RNA template of FIG. 42, the primer or ITR sequence is a self-priming nucleic acid sequence based upon an adenovirus ITR such as provided by SEQ ID NO.: 25 below.

TABLE 36

| | |
|---|---|
| Adenovirus | TTGGCCACTCCCTCTCTGCGCGCTCGCTCGCTCAC |
| ITR DNA | TGAGGCCGCCCGGGCAAAGCCCGGGCGTCGGGCGA |
| Sequence | CCTTTGGTCGCCCGGCCTCAGTGAGCGAGCGAGCG |
| | CGCAGAGAGGGAGTGGCCAA |
| | (SEQ ID NO.: 25) |
| RNA form of | UUGGCCACUCCCUCUCUGCGCGCUCGCUCGCUCAC |
| Adenovirus | UGAGGCCGCCCGGGCAAAGCCCGGGCGUCGGGCGA |
| ITR Sequence | CCUUUGGUCGCCCGGCCUCAGUGAGCGAGCGAGCG |
| | CGCAGAGAGGGAGUGGCCAA |
| | (SEQ ID NO.: 26) |

The RNA form of the ITR DNA sequence basically substitutes uracil for thymine as suggested by SEQ ID NO.: 26. In the FIG. 42 template, either SEQ ID No. 25 or 26 may serve as the 5′ and 3′ ITR, i.e., either the DNA or RNA form may act as the ITR. Although the ITR (or self-priming nucleic acid) sequence of SEQ ID NOS.: 25 and 26 are 124 bases, other embodiments of the self-priming nucleic acid sequence could be a lesser number of consecutive bases from SEQ ID NO.: 25 or 26. For example any number of consecutive bases from SEQ ID NO.: 25 or 26 between 10 and 120 bases, e.g., at least 10, 30, 60, 90, or 120, consecutive bases from SEQ ID NO.: 25 or 26. In preferred embodiments, the self-priming nucleic acid sequence is the same at the 3′ end and the 5′ ends of the RNA template (i.e., reading from the 5′ to the 3′ ends of the self-priming sequence). Similarly, the self-priming nucleic acid sequence could be any virtually any ITR sequence. In many examples, the ITR serving as the self-priming sequence has a length of between 10 and 3000 bases (or any sub-range in between) and more preferably between 10 and 500 bases. Although SEQ ID No. 25 is the ITR from one member of the adenovirus family, the ITR could come from other members of the adenovirus family.

The UTR sequences of the template can play a role in promoting stability and/or translation efficiency in the transcription process. The RNA preferably has 5′ and 3′ UTRs. The length of the 3′ UTR can be 100 nucleotides or less, or exceed 100 nucleotides. In some embodiments RNA with 3′ UTR greater than 100 nucleotides are translated more efficiently than RNA with less than 100 nucleotides of 3′ UTR. In some embodiments the 3′ UTR sequence is between 100 and 5000 nucleotides (or any sub-range in between). The length of the 5′ UTR is not as critical as the length of the 3′ UTR and can be shorter. In one embodiment, the 5′ UTR is between zero and 3000 nucleotides in length (or any sub-range in between). The length of 5′ and 3′ UTR sequences to be added to the coding region can be altered by different methods, including, but not limited to, PCR. Using this approach, one of ordinary skill in the art can modify the 5′ and 3′ UTR lengths required to achieve optimal translation efficiency following transfection of the transcribed RNA. See for example, “Optimization of mRNA Untranslated Regions for Improved Expression of Therapeutic mRNA,” RNA Biology 2018, Vol. 15, No. 6, 756-762, which is incorporated by reference herein.

The 5′ and 3′ UTR can be heterologous UTRs. In some embodiments, the UTR are derived from the host gene, such as an actively expressed host gene, to ensure sufficient translation of the RNA in the host. UTR sequences that are not endogenous can be added by incorporating the UTR sequences by PCR or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain a Kozak sequence. For example, when a 5′ UTR that is not endogenous to the gene of interest is being added by PCR as described above, a consensus Kozak sequence can be included, or redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the RNA.

In one embodiment, UTR sequence could be the SEQ ID NOS. 26 and 27 below:

TABLE 37

| | |
|---|---|
| 5′ UTR | AAAUAAGAGAGAAAAGAAGAGUA AGAAGAAAUAUAAG |
| | AGCCACC (SEQ ID NO.: 27) |
| 3′ UTR | GCUGCCUUCUGCGGGGCUUGCCUUCUGGCCAUGCCCUUCU |
| | UCUCUCCCUUGCACCUGUACCUCUUGGUCUUUGAAUAAAG |
| | CCUGAGUAGGAAG (SEQ ID NO.: 28) |

Other embodiments could include 5′ and 3′ UTR sequences at least 50% homogenous with SEQ ID NOS.: 27 and 28.

In general, the promoter in the FIG. 42 template may be any sequence encoding an eukaryotic promoter capable of regulating expression of the gene in the template. In the specific example of FIG. 24, the promoter is EFα1 to regulate the illustrated gene EGFP. But other examples of promoters have been described above. Certain more preferred promoters may include CMV, EF1, EFα1, SV40, human PGK1, mouse PGK1, Ubc, human beta actin, chicken beta actin, CAG, TRE, UAS, Ac5, Polyhedrin, CaMKIIa, GAL1.10, TEF1, GDS, ADH1, CaMV35S, Ubi, HI, or U6. Any one of these promoters may generally be employed to successfully regulate many different genes. For example, EFα1 is a promoter sequence that will successfully regulate many different genes, particularly those genes listed below. However, those skilled in the art will also recognize that routine experimentation may allow more optimized gene/promoter pairing.

In many embodiments, the FIG. 42 RNA template will include a sequence encoding a gene which expresses a polypeptide or polynucleotide therapeutic to treatment of HIV. In one embodiment, the gene sequence encodes at least one an immunogenic peptide or protein from the group consisting of influenza, VZV (chicken pox or zoster), Herpes Simplex Virus (HSV), (Human Immunodeficiency Virus (HIV), Hepatitis B Virus (HBV), Hepatitis C Virus (HCV), Measles, Mumps Rubella, Cytomegalovirus (CMV), Poliovirus, Epstein Barr Virus (EBV), and Rotavirus. In another embodiment, the gene sequence encodes at least one a bacterial immunogen from the group consisting of streptococcus, clostridia, and neisseria. In a still further embodiment, the gene sequence encodes at least one from the group consisting of a zinc finger nuclease (ZFN), a Transcription Activator-Like Effector Nuclease (TALEN), a Cas 9 enzyme, a Cpf1 enzyme, an RNase inhibitor, VZV IE62, and Influenza Nucleoprotein. Most generally, the gene sequence could encode at least one from the group consisting of peptides, proteins, enzymes, antibodies, immunologically relevant proteins or peptide, short fragment RNA, short fragment DNA, ribozymes, and gene-editing enzymes and be employed in treating conditions other than HIV. Likewise, diagnostic or marker genes could be employed in the RNA template, for example EGFP. Those skilled in the art will recognize that the sequence for genes are often represented in their DNA format. The RNA format of the gene sequence is generally the same as the DNA, except the thymine bases are replaced with uracil.

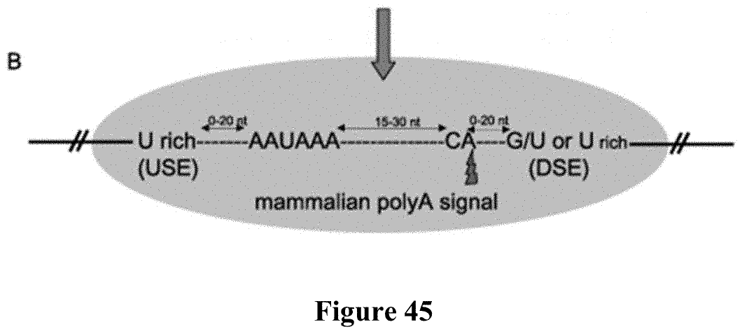

The FIG. 42 RNA template also will include a Poly A signal sequence (i.e., encoding Poly A sequence) between the 3′ UTR sequence and the 3′ self-priming sequence. Any number of different poly A signal sequences may be used, including mammalian poly A signal sequences in the format of FIG. 45, which is described in more detail in “Ending the Message: Poly(A) Signals Then and Now,” Genes & Development, 25:1770-1782 (2011), which is incorporated by reference herein. In certain embodiments, the Poly A sequence is anywhere between 10 bases and 400 bases (or any sub-range in between). In one preferred embodiment, the Poly A signal sequence is an SV 40 Poly A signal sequence as seen in Table 38:

TABLE 38

| | |
|---|---|
| SV 40 Poly A | AAUAAAGCAAUAGCAUCACAAAUUUCACAAAU |
| Signal Sequence | AAA (SEQ ID NO.: 29) |

As suggested in previously described embodiments, the RNA template will often be combined with an aviral delivery vector, for example by encapsulating the RNA template or by forming a complex with the RNA template. Examples of delivery vectors which may encapsulate the RNA template include liposomes and lipid nanoparticle (LNP) systems. LNP systems are currently one of the lead non-viral delivery systems for the clinical delivery of genetic materials. Such LNP systems are described in publications such as US Published Application No. 2017/0210697, “Compounds And Compositions For Intracellular Delivery Of Therapeutic Agents,” and “Lipid Nanoparticle Systems for Enabling Gene Therapies,” Mol Ther. 2017 Jul. 5; 25(7): 1467-1475, both of which are incorporated by reference herein. Examples of delivery vectors which form complexes with the RNA template include Polyethylenimines or PEIs. PEI condenses RNA/DNA into positively charged particles, which bind to anionic cell surface residues and are brought into the cell via endocytosis. Once inside the cell protonation of the amines results in an influx of counter-ions and a lowering of the osmotic potential. Osmotic swelling results and bursts the vesicle releasing the polymer-RNA/DNA complex (polyplex) into the cytoplasm. If the polyplex unpacks then the RNA/DNA is free to diffuse to the nucleus. PEI vectors are commercially available from companies such as Polyplus-Transfection of Illkirch, France, which provides in vivo-jetPEI®. Other examples of delivery vectors forming complexes include synthetic transfection reagents such as Viromer® available from Lipocalyx GmbH of Halle (Saale), Germany. Moreover, the RNA and DNA segments described herein may often be directly administered to the patient as “naked” or “wild type” genetic materials, or with minor chemical modifications to enhance the RNA/DNA segments ability to remain associated in the bloodstream until reaching the target cells. In certain embodiments, the delivery vectors may be combined with a ligand to make the delivery vector selective for specific cells, e.g., T-cells, macrophages, and monocytes. However, in other embodiments, the delivery vectors need not be target to specific cells, but may be allowed to generally deliver the RNA template to the cells of the patient, since the RNA template will only be transcribed into DNA in cells containing reverse transcriptase. In many method embodiments, the delivery vectors are employed to administer to a patient a “therapeutically effective amount” of the RNA template and/or DNA. In these embodiments, the therapeutic amount may vary, in milligrams per kilogram of body weight, from about 0.01 mg/kg to 1000 mg/kg (or any sub-range in between) of RNA and/or DNA. In one embodiment, the therapeutically effective amount is at least 0.1 mg/kg, but could be also be at least 1 mg/kg, at least 10 mg/kg, etc.

FIG. 42 also suggests the process within cells containing reverse transcriptase whereby the RNA template is synthesized into DNA and ultimately expresses the gene in the RNA template. Initially, the aviral RNA template serves as a self-primed template for the synthesis of a first single stranded DNA complementary to the RNA template by the reverse transcriptase process. Next, the first single stranded DNA serves as a template for a second single stranded DNA complementary to the first single stranded DNA via a DNA polymerase process. The first and second single stranded DNA then hybridizes to form double stranded DNA. Finally, the double stranded DNA will be acted upon by RNA polymerase to create messenger RNA which synthesizes the polypeptide or polynucleotide expressed by the gene and therapeutic to treatment of HIV.

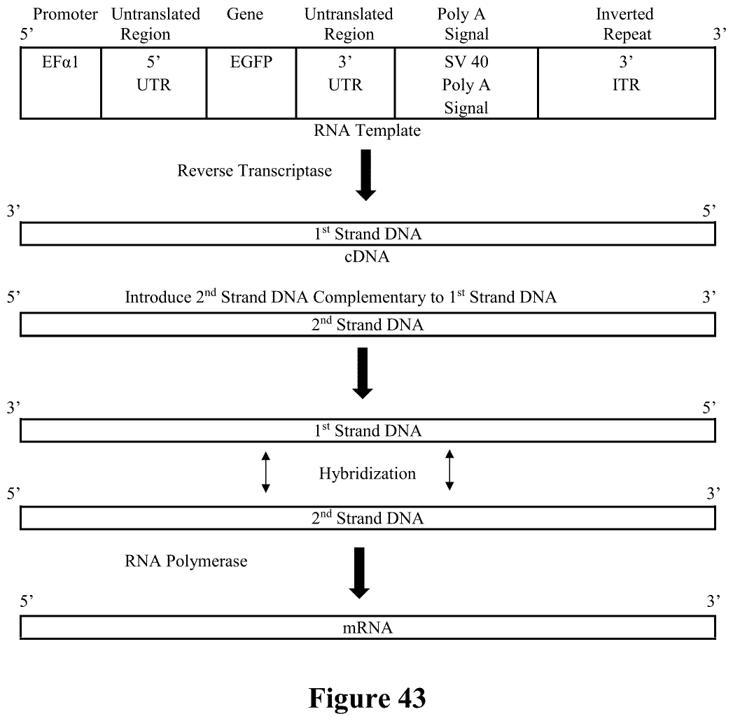

FIG. 43 illustrates a further embodiment of an RNA template which may be employed in another method of treating disorders involving cells exhibiting reverse transcriptase. The method differs from that described in reference to FIG. 42 in that not only an RNA template is delivered to the cell, but also a single stranded DNA as explained below. Viewing FIG. 43, the RNA template is similar to that in FIG. 42, but lacks the self-priming sequence at the 5′ end. The self-priming nucleic acid sequence at the 3′ end of the RNA template may be at least 10 consecutive bases from SEQ ID NO.: 26, but may also be another self-priming sequence. Similarly, the 5′ and 3′ UTRs, the promoter, the gene, and the poly A sequences may be those described in reference to the FIG. 42 RNA template.

The aviral RNA template will be encapsulated or complexed with a delivery vector as described in reference to the FIG. 42 RNA template. It will be understood that once delivered to the cells expressing reverse transcriptase, the RNA template will serve as the basis for the synthesis of only a first single stranded DNA complementary to the RNA template since there is a self-priming sequence only on the 3′ end. However, the method associated with FIG. 43 also involves delivery of a second single stranded DNA to the cells expressing reverse transcriptase, i.e., this second single stranded DNA is not necessarily synthesized within the human to whom the RNA template is administered. This second single stranded DNA will generally be complementary to the sequences in the first single stranded DNA which correspond to the regions of the RNA template. There may be embodiments where the second single stranded DNA is complementary only to the regions of the first single stranded DNA which encodes the regions of (i) the gene and (ii) the eukaryotic promoter from the RNA template. In still other embodiments, the second single stranded DNA is at least 90% (or any percentage between 90 and 100) complementary to the relevant regions of the first single stranded DNA. Other regions of the second single strand DNA (e.g., UTR Poly A, or primer sequences) may or may not have any complementary relationship to the corresponding regions of the first single strand DNA.

As suggested above, both the RNA template and the second single stranded DNA will be delivered to cells of the patient. The delivery vectors may be same as described in reference to FIG. 42. In one embodiment, both the RNA template and the second single stranded DNA may be encapsulated in or complexed with the same aviral delivery vector. Or in other embodiments, the RNA template can be encapsulated or complexed with one delivery vector, while the second single stranded DNA is encapsulated or complexed with a separate delivery vector. Stated differently, either (i) both the RNA template and the second single stranded DNA are associated with the same delivery vector, or (ii) the RNA template and the second single stranded DNA each are associated with separate delivery vectors. When the RNA template and second single stranded DNA are associated with separate delivery vectors, the RNA template and second single stranded DNA can be administered to the patient at the same time or at different times.

When the RNA template is administered to the patient, it will synthesize a first single stranded DNA in cells expressing reverse transcriptase (but not in cells lacking reverse transcriptase). Regardless of whether the second single stranded DNA is introduced to the cells with the same delivery vector as the RNA template or a separate delivery vector, the second single stranded DNA should likewise be present in the cells expressing reverse transcriptase. This will allow the first single stranded DNA and the second single stranded DNA to at least partially hybridize to form a double stranded DNA. Again, this double stranded DNA will be the source of the messenger RNA synthesizing the polypeptide or polynucleotide therapeutic to treatment of HIV (or other conditions being treated by the expressed gene).

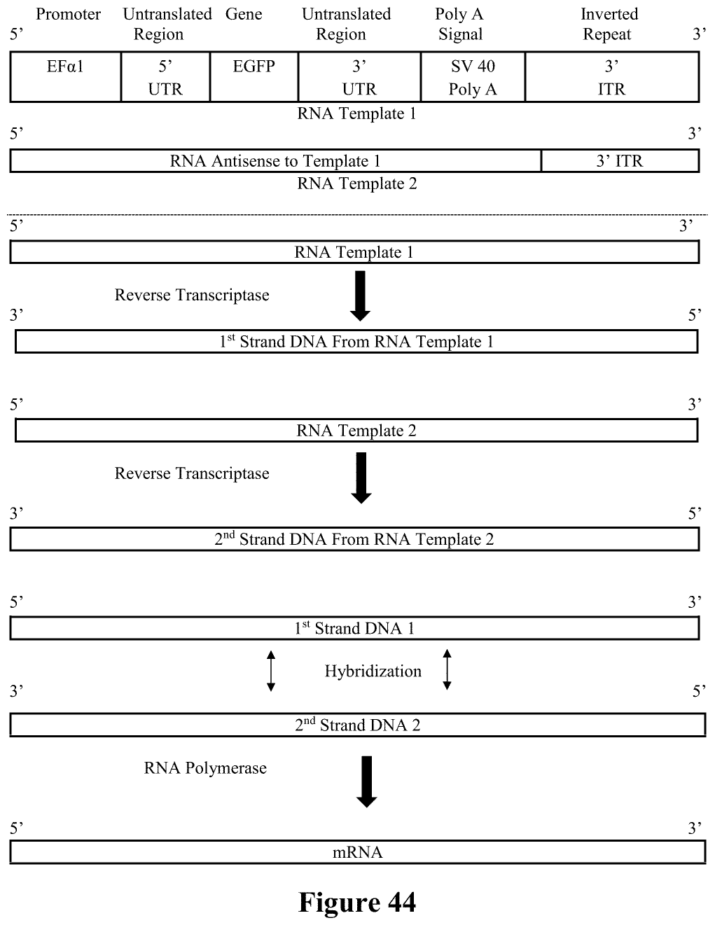

FIG. 44 illustrates a further embodiment of an RNA template which may be employed in another method of treating disorders involving cells exhibiting reverse transcriptase. The method associated with FIG. 44 differs from that described in reference to FIG. 42 or FIG. 43 in that two related RNA templates are delivered to the cells as explained below. Viewing FIG. 44, a first RNA template may be the same as that described in FIG. 43, including the lack of the self-priming sequence at the 5′ end.

However, rather than delivering a second single stranded DNA to the cells expressing reverse transcriptase, the method associated with FIG. 44 delivers a second RNA template to the cell. As suggested in FIG. 44, the second RNA template will be the anti-sense sequence to the first RNA template, exclusive of the self-priming nucleic acid sequence. In other words, the self-priming 3′ region of both RNA templates will be substantially the same. In more practical embodiments, the second RNA template only need to be at least 90% the anti-sense of the first RNA template (again exclusive of the self-priming 3′ region). The first and second RNA templates will be encapsulated in or complexed with delivery vectors similar to the techniques described in reference to FIG. 42. Likewise, the delivery methods may include either (i) both the first RNA template and the second RNA template being associated with the same delivery vector, or (ii) the first RNA template and the second RNA template each being associated with separate delivery vectors.

When both RNA templates are present in a cell expressing reverse transcriptase, the first RNA template serves as a template for the synthesis of a first single stranded DNA complementary to the first RNA template, while the second RNA template serves as a template for the synthesis of a second single stranded DNA complementary to the second RNA template. Thereafter, the first and second single stranded DNAs are able to at least partially hybridize to form a double stranded DNA.

While certain embodiments of the RNA template described above list the self-priming or ITR sequence as a variant of Seq. ID No. 26, other embodiments could employ self-priming sequences unrelated to Seq. ID no. 26. It will be understood that cDNA synthesis is independent of any primer addition. This property may be general to all RNAs and is not associated with small free nucleic acids, such as tRNAs and microRNAs. Rather, it corresponds to initiation of cDNA synthesis from the 3′ end of an RNA template, and a model is proposed in which the template RNA snaps back upon itself and creates a transient RNA primer suitable for the Reverse Transcription. In the present disclosure, a self-priming RNA sequence is an ITR at the 3′ end which snaps back on itself and serves as a primer for synthesis of the cDNA. Any of the RNA templates contemplated by this disclosure could employ such a self-priming sequence in place of the Seq. ID No. 26 identified in the specific examples Likewise, the self-priming sequences at the 3′ and 5′ ends do not need to be similar or related sequences.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

1. A method for treating HIV in a human having cells expressing reverse transcriptase comprising administering to the human a therapeutically effective amount of a composition comprising:
(a) an aviral RNA template comprising:
(i) a self-priming nucleic acid sequence at the 3′ end and the 5′ end of the RNA template, the self-priming nucleic acid including at least 10 consecutive bases from SEQ ID NO.: 25 or 26;
(ii) a sequence encoding a gene which expresses a polypeptide or polynucleotide therapeutic to treatment of HIV;
(iii) a sequence encoding an eukaryotic promoter capable of regulating expression of the gene;
(b) an aviral delivery vector encapsulating or forming a complex with the RNA template;
(c) wherein the aviral RNA template serves as a self-primed template for the synthesis of a first single stranded DNA complementary to the RNA template by reverse transcriptase in cells expressing reverse transcriptase;
(d) wherein the first single stranded DNA is not synthesized from the aviral RNA template in cells lacking reverse transcriptase; and
(e) wherein the first single stranded DNA serves as a template for a second strand of DNA complementary to the first single stranded DNA.

2. The method of treating HIV of claim 1, wherein the self-priming nucleic acid including at least 20, 30, 50, 70, 90, or 110 consecutive bases from SEQ ID NO.: 25 or 26;

3. The method of treating HIV of claim 2, wherein the self-priming nucleic acid includes SEQ ID NO.: 25 or 26;

4. The method of treating HIV of claim 3, wherein the promoter is at least one from the group consisting of EF1, CMV, EF1a, SV40, human PGK1, mouse PGK1, Ubc, human beta actin, chicken beta actin, CAG, TRE, UAS, Ac5, Polyhedrin, CaMKIIa, GAL1.10, TEF1, GDS, ADH1, CaMV35S, Ubi, HI, and U6.

5. The method of treating HIV of claim 4, wherein the gene sequence encodes at least one an immunogenic peptide or protein from the group consisting of influenza, VZV (chicken pox or zoster), Herpes Simplex Virus (HSV), (Human Immunodeficiency Virus (HIV), Hepatitis B Virus (HBV), Hepatitis C Virus (HCV), Measles, Mumps Rubella, Cytomegalovirus (CMV), Poliovirus, Epstein Barr Virus (EBV), and Rotavirus.

6. The method of treating HIV of claim 4, wherein the gene sequence encodes at least one a bacterial immunogen from the group consisting of streptococcus, clostridia, and neisseria.

7. The method of treating HIV of claim 4, wherein the gene sequence encodes at least one from the group consisting of a ZFN, a TALEN, a Cas 9 enzyme, a Cpf1 enzyme, an RNase inhibitor, VZV IE62, and Influenza Nucleoprotein.

8. The method of treating HIV of claim 4, wherein the gene sequence encodes at least one from the group consisting of peptides, proteins, enzymes, antibodies, immunologically relevant proteins or peptide, short fragment RNA, short fragment DNA, ribozymes, and gene-editing enzymes.

9. The method of treating HIV of claim 5, wherein the vector is one from the group consisting of a liposome, a lipid nanoparticle system, a polyethylenimine, or a synthetic transfection agent.

10. The method of treating HIV of claim 1, wherein the aviral RNA template comprises, ordered from the 5′ to the 3′ end:
(i) a self-priming nucleic acid sequence at the 5′ end of the RNA template, the self-priming nucleic acid including at least 10 consecutive bases from SEQ ID NO.: 25 or 26;
(ii) a sequence encoding an eukaryotic promoter capable of regulating expression of a gene;
(iii) a 5′ UTR sequence;
(iv) a sequence encoding the gene, wherein the gene expresses a polypeptide or polynucleotide therapeutic to HIV;
(v) a 3′ UTR sequence;
(vi) a poly A signal sequence; and
(vii) a self-priming nucleic acid sequence at the 3′ end of the RNA template, the self-priming nucleic acid including at least 10 consecutive bases from SEQ ID NO.: 25 or 26.

11. The method of treating HIV of claim 1, further comprising nonobligatory sequences between one or more of the sequences in 10(i) to 10(vii).

12. The method of treating HIV of claim 1, wherein the aviral RNA template comprises, ordered from the 5′ to the 3′ end:
(i) the self-priming nucleic acid sequence at the 5′ end of the RNA template is SEQ ID NO.: 26;
(ii) the eukaryotic promoter is EFα1;
(iii) a 5′ UTR sequence consisting of SEQ ID No: 27;
(iv) the sequence encodes the gene Influenza Nucleoprotein;
(v) a 3′ UTR sequence consisting of SEQ ID No: 28;
(vi) a poly A signal sequence consisting of SEQ ID No: 29;
(vii) the self-priming nucleic acid sequence at the 3′ end of the RNA template is SEQ ID NO.: 26; and
(viii) the aviral delivery vector is a lipid nanoparticle system administered in an amount of at least 0.1 mg/kg of the RNA template.

13-32. (canceled)

33. A method for treating HIV in a human having cells expressing reverse transcriptase comprising administering to the human a therapeutically effective amount of a composition comprising:
(a) an aviral RNA template comprising:
(i) a self-priming nucleic acid sequence at the 3′ end and the 5′ end of the RNA template, the self-priming nucleic acid sequence being an inverted repeat sequence including a first set of nucleotides followed by a second set which are the reverse complement of the first set;
(ii) a sequence encoding a gene which expresses a polypeptide or polynucleotide therapeutic to treatment of HIV;
(iii) a sequence encoding an eukaryotic promoter capable of regulating expression of the gene;
(b) an aviral delivery vector encapsulating or forming a complex with the RNA template;
(c) wherein the aviral RNA template serves as a self-primed template for the synthesis of a first single stranded DNA complementary to the RNA template by reverse transcriptase in cells expressing reverse transcriptase;
(d) wherein the first single stranded DNA is not synthesized from the aviral RNA template in cells lacking reverse transcriptase; and
(e) wherein the first single stranded DNA serves as a template for a second strand of DNA complementary to the first single stranded DNA.

34. The method of claim 33, wherein the first set of nucleotides is between 5 and 200 bases.

35. The method of claim 34, further including a third set of non-complementary nucleotides between the first set and the second set.

36. The method of claim 34, wherein the self-priming nucleic acid sequence is SEQ ID NO.: 25 or 26.

37. The method of claim 34, wherein the self-priming nucleic acid sequence is a palindromic sequence.

38. (canceled)

39. A method for treating HIV in a human having cells expressing reverse transcriptase comprising administering to the human a therapeutically effective amount of a composition comprising:
(a) a first aviral RNA template comprising:
(i) a self-priming nucleic acid sequence at the 3′ end of the RNA template, the self-priming nucleic acid sequence being an inverted repeat sequence including a first set of nucleotides followed by a second set which are the reverse complement of the first set;
(ii) a sequence encoding a gene which expresses a polypeptide or polynucleotide therapeutic to treatment of HIV;
(iii) a sequence encoding an eukaryotic promoter capable of regulating expression of the gene;
(iv) wherein the first aviral RNA template serves as a template for the synthesis of a first single stranded DNA complementary to the first aviral RNA template synthesized in cells expressing reverse transcriptase;
(b) a second aviral RNA template comprising:
(i) a self-priming nucleic acid sequence at the 3′ end of the RNA template, the self-priming nucleic acid sequence being an inverted repeat sequence including a first set of nucleotides followed by a second set which are the complement of the first set;
(ii) a sequence at least 90% anti-sense to the first aviral RNA template, exclusive of the self-priming nucleic acid sequence of the first aviral RNA template;
(ii) wherein the second aviral RNA template serves as a template for the synthesis of a second single stranded DNA complementary to the second aviral RNA template synthesized in cells expressing reverse transcriptase;
(c) an aviral delivery vector encapsulating or forming a complex with either (i) both the first and second aviral RNA templates being associated with the same delivery vector, or (ii) the first and second aviral RNA templates each being associated with separate delivery vectors;
(d) wherein the first single stranded DNA of (a)(iv) and the second single stranded DNA of (b)(ii) at least partially hybridize to form a double stranded DNA.

40-124. (canceled)


SEQUENCE LISTING (27 sequences)

SEQ ID NO: 1 - DNA, 19 bp, Artificial Sequence
  Synthetic
ctacctgagcacccagtcc

SEQ ID NO: 2 - DNA, 23 bp, Artificial Sequence
  Synthetic
ggaaataagagagaaaagaagag

SEQ ID NO: 3 - DNA, 21 bp, Artificial Sequence
  Synthetic
cttcctactcaggctttattc

SEQ ID NO: 4 - RNA, 18 nt, Artificial Sequence
  Synthetic
uggcgcccgaacagggac

SEQ ID NO: 5 - DNA, 18 bp, Artificial Sequence
  Synthetic
accgcgggcttgtccctg

SEQ ID NO: 6 - DNA, 2322 bp, Artificial Sequence
  Synthetic
gaattctaatacgactcactatagggtccctgttcgggcgccatctagagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaataagagagaaaagaagagtaagaagaaatataagagccaccatggtgagcaagggcgaggagctgttcaccgggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttaattaagctgccttctgcggggcttgccttctggccatgcccttcttctctcccttgcacctgtacctcttggtctttgaataaagcctgagtaggaagaataaagcaatagcatcacaaatttcacaaataaatggcgcccgaacagggacagagaccaagctt

SEQ ID NO: 7 - DNA, 4952 bp, Artificial Sequence
  Synthetic
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctaatacgactcactatagggtccctgttcgggcgccatctagagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttaattaagctgccttctgcggggcttgccttctggccatgcccttcttctctcccttgcacctgtacctcttggtctttgaataaagcctgagtaggaagaataaagcaatagcatcacaaatttcacaaataaatggcgcccgaacagggacagagaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 2286 bp, Artificial Sequence
  Synthetic
gggtcctcgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgttcgggcgccatctagagagtaattcatacaaaaggactcgcccctgcctgtgaaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgtggggaatcccagggaccgtcgttaaactcccactaacgtagaacccagagatcgctgcaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgaagcggccgcttaattaagctgccttctgcggggcttgccttctggccatgcccttcttctctcccttgcacctgtacctcttggtctttgaataaagcctgagtaggaagaataaagcaatagcatcacaaatttcacaaataaatggcgcccgaacagggac

SEQ ID NO: 9 - DNA, 18 bp, Artificial Sequence
  Synthetic
gtccctgttcgggcgcca

SEQ ID NO: 10 - RNA, 18 nt, Artificial Sequence
  Synthetic
gucccuguucgggcgcca

SEQ ID NO: 11 - DNA, 35 bp, Artificial Sequence
  Synthetic
tttatttgtgaaatttgtgatgctattgctttatt

SEQ ID NO: 12 - DNA, 35 bp, Artificial Sequence; identical to SEQ ID NO: 11
  Synthetic

SEQ ID NO: 13 - DNA, 20 bp, Artificial Sequence
  Synthetic
gggcgccatctagagagtaa

SEQ ID NO: 14 - DNA, 20 bp, Artificial Sequence
  Synthetic
gggcgccatttatttgtgaa

SEQ ID NO: 15 - DNA, 20 bp, Artificial Sequence
  Synthetic
tgaattacttccacgcccct

SEQ ID NO: 16 - DNA, 20 bp, Artificial Sequence
  Synthetic
cacatgaagcagcacgactt

SEQ ID NO: 17 - DNA, 20 bp, Artificial Sequence
  Synthetic
aagtcgtgctgcttcatgtg

SEQ ID NO: 18 - DNA, 20 bp, Artificial Sequence
  Synthetic
cttcagtctccacccactca

SEQ ID NO: 19 - DNA, 19 bp, Artificial Sequence
  Synthetic
ggccagcttgagactaccc

SEQ ID NO: 20 - DNA, 20 bp, Artificial Sequence
  Synthetic
aggggcgtggaagtaattca

SEQ ID NO: 21 - DNA, 2225 bp, Artificial Sequence
  Synthetic
ttcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttaattaagctgccttctgcggggcttgccttctggccatgcccttcttctctcccttgcacctgtacctcttggtctttgaataaagcctgagtaggaagaataaagcaatagcatcacaaat

SEQ ID NO: 22 - DNA, 2225 bp, Artificial Sequence; identical to SEQ ID NO: 21
  Synthetic

SEQ ID NO: 23 - DNA, 125 bp, Artificial Sequence
  Synthetic
ttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 24 - RNA, 125 nt, Artificial Sequence
  Synthetic
uuggccacucccucucugcgcgcucgcucgcucacugaggccgcccgggcaaagcccgggcgucgggcgaccuuuggucgcccggccucagugagcgagcgagcgcgcagagagggaguggccaa

SEQ ID NO: 25 - DNA, 44 bp, Artificial Sequence
  Adenovirus
aaauaagagagaaaagaagaguaagaagaaauauaagagccacc

SEQ ID NO: 26 - RNA, 93 nt, Artificial Sequence
  Synthetic
gcugccuucugcggggcuugccuucuggccaugcccuucuucucucccuugcaccuguaccucuuggucuuugaauaaagccugaguaggaag

SEQ ID NO: 27 - RNA, 35 nt, Artificial Sequence
  Synthetic
aauaaagcaauagcaucacaaauuucacaaauaaa